(19) United States
(12) Patent Application Publication
(10) Pub. No.: US 2024/0336572 A1
(43) Pub. Date: Oct. 10, 2024

(54) SUBSTITUTED INDAZOLE PROPIONIC ACID DERIVATIVE COMPOUNDS AND USES THEREOF

(71) Applicant: Pfizer Inc., New York, NY (US)

(72) Inventors: Samit Kumar Bhattacharya, Bedford, MA (US); Edward Lee Conn, Griswold, CT (US); David Christopher Ebner, Newton, MA (US); Brian Stephen Gerstenberger, Cambridge, MA (US); Chan Woo Huh, East Lyme, CT (US); Daniel Wei-Shung Kung, Salem, CT (US); Alan Martin Mathiowetz, Waltham, MA (US); Jessica Gloria Katherine O'Brien, Cambridge, MA (US); Meihua Mike Tu, Acton, MA (US); Kimberly O'Keefe Cameron, Niantic, CT (US); Dilinie Prasadhini Fernando, Jamaica Plain, MA (US); Kevin James Filipski, Reading, MA (US); Esther Cheng Yin Lee, Newtown, MA (US); Sarah Jane Mear, East Lyme, CT (US); Aaron Christopher Smith, Golden, CO (US)

(73) Assignee: Pfizer Inc., New York, NY (US)

(21) Appl. No.: 18/626,467

(22) Filed: Apr. 4, 2024

Related U.S. Application Data
(60) Provisional application No. 63/494,598, filed on Apr. 6, 2023.

(51) Int. Cl. C07D 231/56; A61K 31/416

(52) U.S. Cl. CPC C07D 231/56; A61K 31/416

(57) ABSTRACT

The invention relates to substituted indazole propionic acid derivatives, pharmaceutically acceptable salts, tautomers, or pharmaceutically acceptable salts of the tautomers thereof that can activate adenosine 5′-monophosphate-activated protein kinase (AMPK). The invention further relates to pharmaceutical compositions comprising AMPK-activating substituted indazole propionic acid derivatives, pharmaceutically acceptable salts, tautomers, or pharmaceutically acceptable salts of the tautomers thereof and at least one pharmaceutically acceptable excipient, and methods of treating a condition comprising administering AMPK-activating substituted indazole propionic acid derivatives, pharmaceutically acceptable salts, tautomers, or pharmaceutically acceptable salts of the tautomers thereof.

### BACKGROUND OF THE INVENTION

Adenosine 5′-monophosphate-activated protein kinase (AMPK) is a highly conserved serine/threonine kinase that functions as a central regulator of energy homeostasis. AMPK has been demonstrated to mediate multiple pathways within intestinal epithelial cells, including direct modulation of substrates involved in tight junction stability, polarity, differentiation, nutrient transport, and autophagy. Strengthening the intestinal barrier could have therapeutic potential for metabolic- and inflammatory-related diseases associated with intestinal permeability or a “leaky gut”. Given the functional attributes of AMPK in energy and tissue homeostasis, there is a need for potent and direct, gut-targeted activators of AMPK to treat conditions associated with AMPK activation.

### SUMMARY OF THE INVENTION

The present invention provides, in part, a compound of Formula (I):

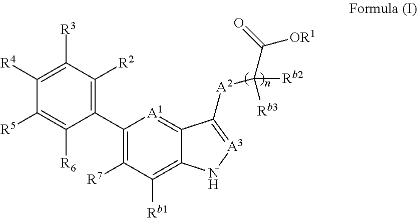

a pharmaceutically acceptable salt, a tautomer, or a pharmaceutically acceptable salt of the tautomer thereof, wherein:
   A¹ is CR⁸, or N;
   A² is CH₂, CHD, CD₂, S, O, or NH;
   A³ is CH, CD, or N;
   R¹ is H, D, C₁₋₈alkyl, C₃₋₆cycloalkyl, or 4-6 membered heterocycloalkyl, each of which is optionally substituted;
   R², R³, R⁵, and R⁶ are each independently H, D, OH, or halogen;
   R⁴ is monocyclic aryl, bicyclic aryl, monocyclic heteroaryl, or bicyclic heteroaryl, each of which is optionally substituted with R⁹, R¹⁰, R¹¹, R¹², or R¹³,
      wherein R⁹, R¹⁰, R¹¹, R¹², and R¹³ are each independently H, D, halogen, CN, oxo, C₁₋₈alkyl, C₃₋₆cycloalkyl, C₀₋₆alkylene-OR^{x}, C₁₋₆haloalkylene-OR^{x}, C₀₋₆alkylene(C₀₋₆haloalkyl)NR^{x}R^{y}, C₁₋₆alkylene(C₁₋₆haloalkyl)NR^{x}R^{y}, 4-6 membered heterocycloalkyl, C(O)OR^{x}, C₀₋₆alkylene-C(O)NR^{x}R^{y}, OC₁₋₃alkylene-heterocycloalkyl, OC₁₋₃alkylene-C(O)NR^{x}R^{y}, O(C₁₋₆alkyl)SO₂NR^{x}NR^{y}, NR^{x}R^{y}, NHSO₂R^{x}, SR^{x}, S—C₁₋₆alkylene-C(O)NR^{x}R^{y}, S(O)R^{x}R^{y}, SO₂R^{x}, SO₂NR^{x}R^{y}, S(O)(NR^{x})R^{y}, S(O)(NR^{x})R^{y}, or SO₂R^{x};
   wherein each R^{x} and R^{y} is independently H, D, C₁₋₆alkyl, C₁₋₆haloalkyl, C₁₋₆alkoxy, C₃₋₆cycloalkyl, C₁₋₆alkylene-amide, OC₀₋₂alkylene-heterocycloalkyl, 4-6 membered heterocycloalkyl, C(O)C₁₋₆alkyl, imino, or C₁₋₆alkylsulfonyl; or R^{x} and R^{y} together with the atoms to which R^{x} and R^{y} are bound can form an optionally substituted ring;
   R⁷ is C₁₋₃alkyl, C₃₋₆cycloalkyl, cyano, or halogen;
   R^{b1}, R^{b2}, and R^{b3} are each independently H or D;
   R^{a} is H, D, or halogen; and
   n is 0, 1, or 2.

Further disclosed herein is 3-[6-chloro-5-(2′-hydroxy[1,1′-biphenyl]-4-yl)-1H-indazol-3-yl]propanoic acid, a pharmaceutically acceptable salt, a tautomer, or a pharmaceutically acceptable salt of the tautomer thereof. The present invention further provides 3-[6-Chloro-5-(2′-hydroxy[1,1′-biphenyl]-4-yl)-1H-indazol-3-yl]propanoic acid. Disclosed herein is a compound of the structure:

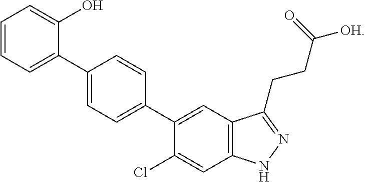 [SMILES: O=C(O)CCc1n[nH]c2cc(Cl)c(-c3ccc(-c4ccccc4O)cc3)cc12]

The present invention provides a method for treating a condition, comprising administering to a subject in need thereof a therapeutically effective amount of the compound of Formula (I), a pharmaceutically acceptable salt, a tautomer, or a pharmaceutically acceptable salt of the tautomer thereof, wherein the condition is an inflammatory condition, an autoimmune condition, or a functional gastrointestinal disorder. The present invention also provides a method for treating a condition, comprising: a) administering to a subject in need thereof a therapeutically effective amount of the compound of Formula (I), a pharmaceutically acceptable salt, a tautomer, or a pharmaceutically acceptable salt of the tautomer thereof; and b) administering a therapeutically effective amount of an additional therapeutic agent.

The present invention further provides a compound of Formula (I), a pharmaceutically acceptable salt, a tautomer, or a pharmaceutically acceptable salt of the tautomer thereof, for use as a medicament. The present invention also provides a compound of Formula (I), a pharmaceutically acceptable salt, a tautomer, or a pharmaceutically acceptable salt of the tautomer thereof, for use in the treatment of an inflammatory condition, an autoimmune condition, or a functional gastrointestinal disorder. The present invention provides use of a compound of Formula (I), a pharmaceutically acceptable salt, a tautomer, or a pharmaceutically acceptable salt of the tautomer thereof, in the manufacture of a medicament for the treatment of an inflammatory condition, an autoimmune condition, or a functional gastrointestinal disorder. The present invention further provides use of a compound of Formula (I), a pharmaceutically acceptable salt, a tautomer, or a pharmaceutically acceptable salt of the tautomer thereof, as a medicament. The present invention further provides use of a compound of Formula (I), a pharmaceutically acceptable salt, a tautomer, or a pharmaceutically acceptable salt of the tautomer thereof, in treating an inflammatory condition, an autoimmune condition, or a functional gastrointestinal disorder.

The present invention provides crystalline 3-[6-Chloro-5-(2′-hydroxy[1,1′-biphenyl]-4-yl)-1H-indazol-3-yl]propanoic acid, a pharmaceutically acceptable salt, a tautomer, or a pharmaceutically acceptable salt of the tautomer thereof.

It is to be understood that both the foregoing general description and the following detailed description are exemplary and explanatory only and are not restrictive of the invention, as claimed.

### BRIEF DESCRIPTION OF THE DRAWINGS

FIG. 1 shows the PXRD of the compound of Example 1, form 1.

### DETAILED DESCRIPTION OF THE INVENTION

The present invention may be understood more readily by reference to the following detailed description of the embodiments of the invention and the Examples included herein. It is to be understood that this invention is not limited to specific synthetic methods of making that may of course vary. It is to be also understood that the terminology used herein is for the purpose of describing specific embodiments only and is not intended to be limiting.

Adenosine 5′-monophosphate-activated protein kinase (AMPK) is a highly conserved serine/threonine kinase that functions as a central regulator of energy homeostasis (Herzig, S. et al. Nat Rev Mol Cell Biol 19:121-135 (2018)). AMPK exists as a heterotrimeric protein complex consisting of a catalytic a-subunit, scaffolding p-subunit, and regulatory y-subunit. Multiple isoforms (e.g., two a, two p, three y) encoded by different genes enable up to twelve possible AMPK heterotrimeric complexes, with each AMPK heterotrimeric complex having a distinct cellular and tissue expression profile. AMPK is activated by upstream kinases, including liver kinase β1 (LKB1) and calcium/calmodulin-dependent protein kinase p (CamKKβ), which phosphorylate the Thr172 active site residue within the a-subunit of AMPK. AMPK is also activated when the ratio of intracellular adenosine monophosphate (AMP):adenosine triphosphate (ATP) or to a lesser extent adenosine diphosphate (ADP):ATP is increased under conditions of energetic stress, such as nutrient starvation, inflammation, and hypoxia (Xiao, B. et al. Nature 449:496-500 (2007)). Upon activation, AMPK phosphorylates direct substrates involved in pathways that promote ATP production (e.g., fatty acid oxidation, glycolysis, glucose uptake, autophagy, and mitophagy) and inhibit ATP consumption (e.g., synthesis of glucose, lipids, and proteins; cell growth) to restore energy balance. Moreover, AMPK can regulate and reprogram metabolism through transcriptional changes by phosphorylating factors that induce or repress gene transcription.

Multiple approaches for pharmacologically activating AMPK exist, both direct and indirect (Kim, J. et al. Exp Mol Med 48:e224 (2016)). 5-aminoimidazole-4-carboxamide-1-β-D-ribofuranoside (AICAR) and metformin increase cytosolic AMP. AICAR functions as an AMP mimetic, whereas metformin indirectly increases cytosolic AMP by inhibiting mitochondrial respiration and the release of ATP. AMP can bind the γ-subunit of AMPK to allosterically activate AMPK. In contrast, direct AMPK agonists can bind the allosteric drug and metabolism (ADaM) site between the α and β subunits of AMPK to activate and protect AMPK from dephosphorylation. Both pan-β and β1-selective AMPK agonists have been described.

AMPK activity can be altered due to pathological conditions, including metabolic and inflammatory diseases, such as obesity, diabetes, cardiovascular disease, and cancer. Additionally, there is evidence that AMPK can promote and maintain intestinal barrier function (Sahoo, S. et al. Nat Commun 12:4246 (2021); Wu, Z. et al. J Cell Physiol 237:3705-3716 (2022)). AMPK has been demonstrated to mediate multiple pathways within intestinal epithelial cells, including direct modulation of substrates involved in tight junction stability, polarity, differentiation, nutrient transport, and autophagy (Sun, X. et al. Open Biol 7:170104 (2017); Rowart, P. et al. Int J Mol Sci 13:2040 (2018); Zhu, M J. et al. Tissue Barrier 6:1-13 (2018); Tsukita, K. et al. Int J Mol Sci 20:6012 (2018)). Strengthening the intestinal barrier could have therapeutic potential for metabolic- and inflammatory-related diseases associated with intestinal permeability or a “leaky gut” (Odenwald, M A. Et al. Clin Gastrenterol Hepatol 11:1075-1083 (2013)). AMPK activators have been developed for systemic administration. Given the functional attributes of AMPK in energy and tissue homeostasis, there is a need for potent and direct, gut-targeted activators of AMPK to treat conditions associated with AMPK activation.

Disclosed herein are AMPK-activating compounds, pharmaceutically acceptable salts, tautomers, or pharmaceutically acceptable salts of the tautomers thereof. Also disclosed herein are AMPK-activating pharmaceutical compositions comprising the AMPK-activating compounds, pharmaceutically acceptable salts, tautomers, or pharmaceutically acceptable salts of the tautomers thereof and at least one pharmaceutically acceptable excipient. Further disclosed herein are methods of synthesizing AMPK-activating compounds or pharmaceutically acceptable salts, tautomers, or pharmaceutically acceptable salts of the tautomers thereof, and methods of administering the AMPK-activating compounds, a pharmaceutically acceptable salt thereof, a tautomer thereof, or a pharmaceutically acceptable salt of the tautomer thereof, in a subject in need thereof to treat a condition. In some embodiments, the AMPK-activating compounds, pharmaceutically acceptable salts, tautomers, or pharmaceutically acceptable salts of the tautomers thereof disclosed herein may be used to treat a metabolic disorder, an inflammatory disorder, an autoimmune disorder, a disorder of gastrointestinal barrier dysfunction, a functional gastrointestinal disorder, a central nervous system disorder, an eating disorder, a nutritional disorder, or an allergy. In a preferred embodiment, the AMPK-activating compounds, pharmaceutically acceptable salts, tautomers, or pharmaceutically acceptable salts of the tautomers thereof disclosed herein may be used to treat a metabolic disorder, an inflammatory disorder, an autoimmune disorder, a disorder of gastrointestinal barrier dysfunction, or a functional gastrointestinal disorder.

### Definitions

Unless otherwise defined herein, scientific and technical terms used in connection with the present invention have the meanings that are commonly understood by those of ordinary skill in the art. The invention described herein suitably may be practiced in the absence of any element(s) not specifically disclosed herein. “Compounds of the invention” or “compounds of the disclosure” include compounds of Formula I, Ia, II, III, IVa-c, and V, and the novel intermediates used in the preparation thereof. One of ordinary skill in the art will appreciate that compounds of the invention include conformational isomers (e.g., cis and trans isomers) and all optical isomers (e.g., enantiomers and diastereomers), racemic, diastereomeric and other mixtures of such isomers, tautomers thereof, where they may exist. One of ordinary skill in the art will also appreciate that compounds of the invention include solvates, hydrates, isomorphs, polymorphs, esters, salt forms, prodrugs, and isotopically labelled versions thereof, where they may be formed.

As used herein, the singular form “a”, “an”, and “the” include plural references unless indicated otherwise. For example, “a” substituent includes one or more substituents.

As used herein, the term “about” when used to modify a numerically defined parameter (e.g., the dose of an AMPK-activating compound, a pharmaceutically acceptable salt, a tautomer, or a pharmaceutically acceptable salt of the tautomer thereof) means that the parameter may vary by as much as 10% below or above the stated numerical value for that parameter. For example, a dose of about 5 mg means 5 mg±10%, i.e., it may vary between 4.5 mg and 5.5 mg.

The term “and/or” means one or more. For example, “X and/or Y” shall be understood to mean either “X and Y” or “X or Y” and shall be taken to provide explicit support for both meanings or for either meaning. Similarly, when more than 2 expressions are listed, such as in “X, Y and/or Z”, it shall be understood to mean either i) “X and Y”, “X, Y and Z”, “X and Z”, or “Y and Z”, or ii) “X or Y or Z” and shall be taken to provide explicit support for all meanings.

Any open valency appearing on a carbon, oxygen, sulfur, or nitrogen atom in the structures disclosed herein indicates the presence of a hydrogen, unless indicated otherwise.

If substituents are described as being “independently selected” from a group, each substituent is selected independent of the other. Each substituent therefore may be identical to or different from the other substituent(s).

“Optional” or “optionally” means that the subsequently described event or circumstance may, but need not occur, and the description includes instances where the event or circumstance occurs and instances in which it does not.

The terms “optionally substituted” and “substituted or unsubstituted” are used interchangeably to indicate that the particular group being described may have no non-hydrogen substituents (i.e., unsubstituted), or the group may have one or more non-hydrogen substituents (i.e., substituted). If not otherwise specified, the total number of substituents that may be present is equal to the number of H atoms present on the unsubstituted form of the group being described. Where an optional substituent is attached via a double bond, such as an oxo (═O) substituent, the group occupies two available valences, so the total number of other substituents that are included is reduced by two. In the case where optional substituents are selected independently from a list of alternatives, the selected groups may be the same or different. Throughout the disclosure, it will be understood that the number and nature of optional substituent groups will be limited to the extent that such substitutions make chemical sense to one of ordinary skill in the art.

“Halogen” or “halo” refers to fluoro, chloro, bromo and iodo (F, Cl, Br, I). In a preferred embodiment, “halo” refers to fluoro. In a preferred embodiment, “halo” refers to chloro.

“Cyano” refers to a substituent having a carbon atom joined to a nitrogen atom by a triple bond, i.e., —C═N.

“Hydroxy” refers to an —OH group.

“Oxo” refers to a double bonded oxygen (═O).

The term C₁-Cₓ includes C₁-C₂, C₁-C₃ . . . C₁-Cₓ. By way of example only, a group designated as “C₁-C₄” indicates that there are one to four carbon atoms in the moiety, i.e., groups containing 1 carbon atom, 2 carbon atoms, 3 carbon atoms, or 4 carbon atoms. For example, “C₁-C₄ alkyl” indicates that there are one to four atom carbons in the alkyl group, i.e., the alkyl group is selected from methyl, ethyl, propyl, isopropyl, n-butyl, isobutyl, sec-butyl, and t-butyl.

The terms “carbocyclic” or “carbocycle” refer to a ring or ring system where the atoms forming the backbone of the ring are all carbon atoms. The term is distinguished from “heterocyclic” rings or “heterocycles” in which the ring backbone contains at least one atom which is different from carbon. In some embodiments, at least one of the two rings of a bicyclic carbocycle is aromatic. In some embodiments, both rings of a bicyclic carbocycle are aromatic.

For example. Carbocycle includes cycloalkyl and aryl. “Alkyl” refers to a saturated, monovalent aliphatic hydrocarbon radical that has a specified number of carbon atoms, including straight chain or branched chain groups. Alkyl groups may contain, but are not limited to, 1 to 12 carbon atoms (“C₁-C₁₂ alkyl”), 1 to 8 carbon atoms (“C₁-C₈ alkyl”), 1 to 6 carbon atoms (“C₁-C₆ alkyl”), 1 to 5 carbon atoms (“C₁-C₅ alkyl”), 1 to 4 carbon atoms (“C₁-C₄ alkyl”), 1 to 3 carbon atoms (“C₁-C₃ alkyl”), or 1 to 2 carbon atoms (“C₁-C₂ alkyl”). Examples include, but are not limited to, methyl, ethyl, n-propyl, isopropyl, n-butyl, sec-butyl, isobutyl, tert-butyl, n-pentyl, isopentyl, neopentyl, n-hexyl, n-heptyl, n-octyl, and the like. Alkyl groups may be optionally substituted, unsubstituted, or substituted, as further defined herein.

The term “haloalkyl” refers to an alkyl group wherein at least one of the hydrogen atoms of the alkyl group has been replaced by at least one of the same or different halogen atoms. For example, “fluoroalkyl” means an alkyl as defined herein substituted with one, two or three fluoro atoms. Exemplary (C)fluoroalkyl compounds include fluoromethyl, difluoromethyl and trifluoromethyl; exemplary (C₂)fluoroalkyl compounds include 1-fluoroethyl, 2-fluoroethyl, 1,1-difluoroethyl, 1,2-difluoroethyl, 1,1,1-trifluoroethyl, 1,1,2-trifluoroethyl, and the like. Examples of fully substituted fluoroalkyl groups (also referred to as perfluoroalkyl groups) include trifluoromethyl (—CF₃) and pentafluoroethyl (—C₂F5).

“Alkoxy” refers to an alkyl group, as defined herein, that is single bonded to an oxygen atom. The attachment point of an alkoxy radical to a molecule is through the oxygen atom. An alkoxy radical may be depicted as alkyl-O— or O(C₁₋ₓalkyl). Alkoxy groups may contain, but are not limited to, 1 to 8 carbon atoms (“C₁-C₈ alkoxy”), 1 to 6 carbon atoms (“C₁-C₆ alkoxy”), 1 to 4 carbon atoms (“C₁-C₄ alkoxy”), or 1 to 3 carbon atoms (“C₁-C₃ alkoxy”). Alkoxy groups include, but are not limited to, methoxy, ethoxy, n-propoxy, isobutoxy, and the like.

“Alkoxyalkyl” refers to an alkyl group, as defined herein, that is substituted by an alkoxy group, as defined herein. “Alkoxyalkyl” may be depicted as C₁₋ₓalkylene-O—C₁₋₇alkyl. Examples include, but are not limited to, CH₃OCH₂— and CH₃CH₂OCH₂—.

“Cycloalkyl” refers to a fully saturated hydrocarbon ring system that has the specified number of carbon atoms, which may be a monocyclic, bridged or fused bicyclic or polycyclic ring system that is connected to the base molecule through a carbon atom of the cycloalkyl ring.

Cycloalkyl groups may contain, but are not limited to, 3 to 12 carbon atoms (“C₃-C₁₂ cycloalkyl”), 3 to 8 carbon atoms (“C₃-C₈ cycloalkyl”), 3 to 6 carbon atoms (“C₃-C₈ cycloalkyl”), 3 to 5 carbon atoms (“C₃-C₈ cycloalkyl”) or 3 to 4 carbon atoms (“C₃-C₄ cycloalkyl”). Representative cycloalkyl rings include cyclopropyl, cyclobutyl, cyclopentyl, cyclohexyl, cycloheptyl, and cyclooctyl. Polycyclic cycloalkyl groups include, for example, adamantanyl, 1,2-dihydronaphthalenyl, 1,4-dihydronaphthalenyl, tetraenyl, decalinyl, 3,4-dihydronaphthalenyl-1(2H)-one, spiro[2.2]pentyl, norbornyl, and bicyclo[1.1.1]pentyl. Cycloalkyl groups may be optionally substituted, unsubstituted or substituted, as further defined herein.

“Cycloalkoxy” refers to a cycloalkyl group, as defined herein, that is single bonded to an oxygen atom. The attachment point of a cycloalkoxy radical to a molecule is through the oxygen atom. A cycloalkoxy radical may be depicted as cycloalkyl-O— or OC₁₋ₓcycloalkyl. Cycloalkoxy groups may contain, but are not limited to, 3 to 8 carbon atoms (“C₃-C₈ cycloalkoxy”), 3 to 6 carbon atoms (“C₃-C₆ cycloalkoxy”), and 3 to 4 carbon atoms (“C₃-C₄ cycloalkoxy”). Representative cycloalkyl rings include cyclopropyl, cyclobutyl, cyclopentyl, cyclohexyl, cycloheptyl, and cyclooctyl. Polycyclic cycloalkyl groups include, for example, adamantanyl, 1,2-dihydronaphthalenyl, 1,4-dihydronaphthalenyl, tetraenyl, decalinyl, 3,4-dihydronaphthalenyl-1(2H)-one, spiro[2.2]pentyl, norbornyl, and bicyclo[1.1.1]pentyl.

“Heterocycloalkyl” refers to a fully saturated ring system containing the specified number of ring atoms and containing at least one heteroatom selected from N, O and S as a ring member, where ring S atoms are optionally substituted by one or two oxo groups (i.e., S(O)_{q}, where q is 0, 1 or 2) and where the heterocycloalkyl ring is connected to the base molecule via a ring atom, which may be C or N. Heterocycloalkyl rings include rings which are spirocyclic, bridged, or fused to one or more other heterocycloalkyl or carbocyclic rings, where such spirocyclic, bridged, or fused rings may themselves be saturated, partially unsaturated or aromatic to the extent unsaturation or aromaticity makes chemical sense, provided the point of attachment to the base molecule is an atom of the heterocycloalkyl portion of the ring system. Heterocycloalkyl rings may contain 1 to 4 heteroatoms selected from N, O, and S(O)_{q} as ring members, or 1 to 2 ring heteroatoms, provided that such heterocycloalkyl rings do not contain two contiguous oxygen or sulfur atoms. Heterocycloalkyl rings may be optionally substituted, unsubstituted or substituted, as further defined herein. Such substituents may be present on the heterocyclic ring attached to the base molecule, or on a spirocyclic, bridged or fused ring attached thereto. Heterocycloalkyl rings may include, but are not limited to, 3-8 membered heterocycloalkyl groups, for example 4-7 or 4-6 membered heterocycloalkyl groups, in accordance with the definition herein. Illustrative examples of heterocycloalkyl rings include, but are not limited to a monovalent radical of oxirane (oxiranyl), thiirane (thiiranyl), aziridine (aziridinyl), oxetane (oxetanyl), thietane (thietanyl), azetidine (azetidinyl), tetrahydrofuran (tetrahydrofuranyl), tetrahydrothiophene (tetrahydrothiophenyl), pyrrolidine (pyrrolidinyl), tetrahydropyran (tetrahydropyranyl), tetrahydrothiopyran (tetrahydrothiopyranyl), piperidine (piperidinyl), 1,4-dioxane (1,4-dioxanyl), 1,4-oxathiarane (1,4-oxathiaranyl), morpholine (morpholinyl), 1,4-dithiane (1,4-dithianyl), piperazine (piperazinyl), thiomorpholine (thiomorpholinyl), oxepane (oxepanyl), thiepane (thiepanyl), azepane (azepanyl), 1,4-dioxepane (1,4-dioxepanyl), 1,4-oxathiepane (1,4-oxathiepanyl), 1,4-oxaazepane (1,4-oxaazepanyl), 1,4-thiazepane (1,4-thiazapanyl), 1,4-diazepane (1,4-diazepanyl), or 1,4-dithepane (1,4-dithiepanyl). Illustrative examples of bridged and fused heterocycloalkyl groups include, but are not limited to a monovalent radical of 1-oxa-5-azabicyclo-[2.2.1]heptane, 3-oxa-8-azabicyclo-[3.2.1]octane, 3-azabicyclo-[3.1.0]hexane, or 2-azabicyclo-[3.1.0]hexane.

“Aryl” refers to monocyclic, bicyclic (e.g., biaryl, fused) or polycyclic ring systems that contain the specified number of ring atoms, in which all carbon atoms in the ring are of sp² hybridization and in which the pi electrons are in conjugation. Aryl groups may contain, but are not limited to, 6 to 20 carbon atoms (“C₆-C₂₀ aryl”), 6 to 14 carbon atoms (“C₆-C₁₄ aryl”), 6 to 12 carbon atoms (“C₆-C₁₂ aryl”), or 6 to 10 carbon atoms (“C₆-C₁₀ aryl”). Fused aryl groups may include an aryl ring (e.g., a phenyl ring) fused to another aryl ring. Examples include, but are not limited to, phenyl, biphenyl, naphthyl, anthracenyl, phenanthrenyl, indanyl, and indenyl. Aryl groups may be optionally substituted, unsubstituted or substituted, as further defined herein.

Similarly, “heteroaryl” or “heteroaromatic” refer to monocyclic, bicyclic (e.g., heterobiaryl, fused) or polycyclic ring systems that contain the specified number of ring atoms and include at least one heteroatom selected from N, O and S as a ring member in a ring in which all carbon atoms in the ring are of sp² hybridization and in which the pi electrons are in conjugation. Heteroaryl groups may contain, but are not limited to, 5 to 20 ring atoms (“5-20 membered heteroaryl”), 5 to 14 ring atoms (“5-14 membered heteroaryl”), 5 to 12 ring atoms (“5-12 membered heteroaryl”), 5 to 10 ring atoms (“5-10 membered heteroaryl”), 5 to 9 ring atoms (“5-9 membered heteroaryl”), or 5 to 6 ring atoms (“5-6 membered heteroaryl”). Heteroaryl rings are attached to the base molecule via a ring atom of the heteroaromatic ring. Thus, either 5- or 6-membered heteroaryl rings, alone or in a fused structure, may be attached to the base molecule via a ring C or N atom. Examples of heteroaryl groups include, but are not limited to, pyrrolyl, furanyl, thiophenyl, pyrazolyl, imidazolyl, isoxazolyl, oxazolyl, isothiazolyl, thiazolyl, triazolyl, oxadiazolyl, thiadiazolyl, tetrazolyl, pyridinyl, pyridizinyl, pyrimidinyl, pyrazinyl, benzofuranyl, benzothiophenyl, indolyl, benzimidazolyl, indazolyl, quinolinyl, isoquinolinyl, purinyl, triazinyl, naphthyridinyl, cinnolinyl, quinazolinyl, quinoxalinyl and carbazolyl. Examples of 5- or 6-membered heteroaryl groups include, but are not limited to, pyrrolyl, furanyl, thiophenyl, pyrazolyl, imidazolyl, isoxazolyl, oxazolyl, isothiazolyl, thiazolyl, triazolyl, pyridinyl, pyrimidinyl, pyrazinyl and pyridazinyl rings. Heteroaryl groups may be optionally substituted, unsubstituted or substituted, as further defined herein. Illustrative examples of monocyclic heteroaryl groups include, but are not limited to a monovalent radical of pyrrole (pyrrolyl), furan (furanyl), thiophene (thiophenyl), pyrazole (pyrazolyl), imidazole (imidazolyl), isoxazole (isoxazolyl), oxazole (oxazolyl), isothiazole (isothiazolyl), thiazolyl (thiazolyl), 1,2,3-triazole (1,2,3-triazolyl), 1,3,4-triazole (1,3,4-triazolyl), 1-oxa-2,3-diazole (1-oxa-2,3-diazolyl), 1-oxa-2,4-diazole (1-oxa-2,4-diazolyl), 1-oxa-2,5-diazole (1-oxa-2,5-diazolyl), 1-oxa-3,4-diazole (1-oxa-3,4-diazolyl), 1-thia-2,3-diazole (1-thia-2,3-diazolyl), 1-thia-2,4-diazole (1-thia-2,4-diazolyl), 1-thia-2,5-diazole (1-thia-2,5-diazolyl), 1-thia-3,4-diazole (1-thia-3,4-diazolyl), tetrazole (tetrazolyl), pyridine (pyridinyl), pyridazine (pyridazinyl), pyrimidine (pyrimidinyl), or pyrazine (pyrazinyl).

Illustrative examples of fused ring heteroaryl groups include, but are not limited to benzofuran (benzofuranyl), benzothiophene (benzothiophenyl), indole (indolyl), benzimidazole (benzimidazolyl), indazole (indazolyl), benzotriazole (benzotriazolyl), pyrrolo[2,3-b]pyridine (pyrrolo[2,3-b]pyridinyl), pyrrolo[2,3-c]pyridine (pyrrolo[2,3-c]pyridinyl), pyrrolo[3,2-c]pyridine (pyrrolo[3,2-c]pyridinyl), pyrrolo[3,2-b]pyridine (pyrrolo[3,2-b]pyridinyl), imidazo[4,5-b]pyridine (imidazo[4,5-b]pyridinyl), imidazo[4,5-c]pyridine (imidazo[4,5-c]pyridinyl), pyrazolo[4,3-d]pyridine (pyrazolo[4,3-d]pyridinyl), pyrazolo[4,3-c]pyridine (pyrazolo[4,3-c]pyridinyl), pyrazolo[3,4-c]pyridine (pyrazolo[3,4-c]pyridinyl), pyrazolo[3,4-b]pyridine (pyrazolo[3,4-b]pyridinyl), isoindole (isoindolyl), indazole (indazolyl), purine (purinyl), indolizine (indolizinyl), imidazo[1,2-a]pyridine (imidazo[1,2-a]pyridinyl), imidazo[1,5-a]pyridine (imidazo[1,5-a]pyridinyl), pyrazolo[1,5-a]pyridine (pyrazolo[1,5-a]pyridinyl), pyrrolo[1,2-b]pyridazine (pyrrolo[1,2-b]pyridazinyl), imidazo[1,2-c]pyrimidine (imidazo[1,2-c]pyrimidinyl), quinoline (quinolinyl), isoquinoline (isoquinolinyl), cinnoline (cinnolinyl), quinazoline (azaquinazoline), quinoxaline (quinoxalinyl), phthalazine (phthalazinyl), 1,6-naphthyridine (1,6-naphthyridinyl), 1,7-naphthyridine (1,7-naphthyridinyl), 1,8-naphthyridine (1,8-naphthyridinyl), 1,5-naphthyridine (1,5-naphthyridinyl), 2,6-naphthyridine (2,6-naphthyridinyl), 2,7-naphthyridine (2,7-naphthyridinyl), pyrido[3,2-d]pyrimidine (pyrido[3,2-d]pyrimidinyl), pyrido[4,3-d]pyrimidine (pyrido[4,3-d]pyrimidinyl), pyrido[3,4-d]pyrimidine (pyrido[3,4-d]pyrimidinyl), pyrido[2,3-d]pyrimidine (pyrido[2,3-d]pyrimidinyl), pyrido[2,3-b]pyrazine (pyrido[2,3-b]pyrazinyl), pyrido[3,4-b]pyrazine (pyrido[3,4-b]pyrazinyl), pyrimido[5,4-d]pyrimidine (pyrimido[5,4-d]pyrimidinyl), pyrazino[2,3-b]pyrazine (pyrazino[2,3-b]pyrazinyl), or pyrimido[4,5-d]pyrimidine (pyrimido[4,5-d]pyrimidinyl).

“Amino” refers to a group —NH₂, which is unsubstituted. Where the amino is described as substituted or optionally substituted, the term includes groups of the form —NR^{x}R^{y}, where each of R^{x} and R^{y} are independently defined as further described herein. For example, “alkylamino” refers to a group —NR^{x}R^{y}, wherein one of R^{x} and R^{y} is an alkyl moiety and the other is H, and “dialkylamino” refers to —NR^{x}R^{y} wherein both of R^{x} and R^{y} are alkyl moieties, where the alkyl moieties have the specified number of carbon atoms (e.g., —NH(C₁-C₄ alkyl) or —N(C₁-C₄ alkyl)₂).

The term “alkylamino” or “aminoalkyl” refer to a radical of the formula —NHR^{x} or —NR^{x}R^{y}, wherein each R^{x} and R^{y} are independently H, an alkyl group, or an alkylene group. For example, “alkylamino” can refer to a group —NR^{x}R^{y}, wherein one of R^{x} and R^{y} is an alkyl moiety and the other is H; and “dialkylamino” can refer to —NR^{x}R^{y}, wherein both of R^{x} and R^{y} are alkyl moieties, where the alkyl moieties have the specified number of carbon atoms (e.g., —NH(C₁-C₄ alkyl) or —N(C₁-C₄ alkyl)₂). In some embodiments, aminoalkyl refers to —NH-alkylene or alkylene-NH-alkylene, wherein each alkyklene is independent substituted or unsubstituted.

The term “pharmaceutically acceptable” means the substance (e.g., the compounds described herein) and any salt thereof, or composition containing the substance or salt of the invention is suitable for administration to a subject or patient.

“Deuterium enrichment factor” as used herein means the ratio between the deuterium abundance and the natural abundance of deuterium, each relative to hydrogen abundance. An atomic position designated as having deuterium typically has a deuterium enrichment factor of, in particular embodiments, at least 1000 (15% deuterium incorporation), at least 2000 (30% deuterium incorporation), at least 3000 (45% deuterium incorporation), at least 3500 (52.5% deuterium incorporation), at least 3500 (52.5% deuterium incorporation at each designated deuterium atom), at least 4000 (60% deuterium incorporation), at least 4500 (67.5% deuterium incorporation), at least 5000 (75% deuterium incorporation), at least 5500 (82.5% deuterium incorporation), at least 6000 (90% deuterium incorporation), at least 6333.3 (95% deuterium incorporation), at least 6466.7 (97% deuterium incorporation), at least 6600 (99% deuterium incorporation), or at least 6633.3 (99.5% deuterium incorporation).

The term “treating”, “treat” or “treatment” as used herein embraces both preventative, i.e., prophylactic, and palliative treatment, i.e., relieve, alleviate, or slow the progression of the patient's disease (or condition) or any tissue damage associated with the disease.

As used herein, the terms, “subject, “individual” or “patient,” used interchangeably, refer to any animal, including mammals. Mammals according to the invention include canine, feline, bovine, caprine, equine, ovine, porcine, rodents, lagomorphs, primates, humans and the like, and encompass mammals in utero. In an embodiment, humans are suitable subjects. Human subjects may be of any gender and at any stage of development.

As used herein, the phrase “therapeutically effective amount” refers to the amount of active compound or pharmaceutical agent that elicits the biological or medicinal response in a tissue, system, animal, individual or human that is being sought by a researcher, veterinarian, medical doctor or other clinician, which may include one or more of the following:
(1) preventing the disease; for example, preventing a disease, condition or disorder in an individual that may be predisposed to the disease, condition or disorder but does not yet experience or display the pathology or symptomatology of the disease;
(2) inhibiting the disease; for example, inhibiting a disease, condition or disorder in an individual that is experiencing or displaying the pathology or symptomatology of the disease, condition or disorder (i.e., arresting (or slowing) further development of the pathology or symptomatology or both); and
(3) ameliorating the disease; for example, ameliorating a disease, condition or disorder in an individual that is experiencing or displaying the pathology or symptomatology of the disease, condition or disorder (i.e., reversing the pathology or symptomatology or both).

### Compounds of the Invention

Disclosed herein are AMPK-activating compounds. In some embodiments, the compounds disclosed herein are pan-AMPK-activating compounds.

The present disclosure provides a compound of Formula (I):

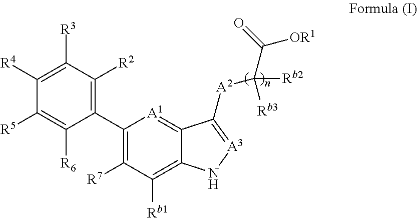

a pharmaceutically acceptable salt, a tautomer, or a pharmaceutically acceptable salt of the tautomer thereof, wherein:
   A¹ is CR⁸, or N;
   A² is CH₂, CHD, CD₂, S, O, or NH;
   A³ is CH, CD, or N;
   R¹ is H, D, C₁₋₈alkyl, C₃₋₆cycloalkyl, or 4-6 membered heterocycloalkyl, each of which is optionally substituted;
   R², R³, R⁵, and R⁶ are each independently H, D, OH, or halogen;
   R⁴ is monocyclic aryl, bicyclic aryl, monocyclic heteroaryl, or bicyclic heteroaryl, each of which is optionally substituted with R⁹, R¹⁰, R¹¹, R¹², or R¹³,
      wherein R⁹, R¹⁰, R¹¹, R¹², and R¹³ are each independently H, D, halogen, CN, oxo, C₁₋₈alkyl, C₃₋₆cycloalkyl, C₀₋₆alkylene-OR^{x}, C₁₋₆haloalkylene-OR^{x}, C₀₋₆alkylene(C₀₋₆haloalkyl)NR^{x}R^{y}, C₁₋₆alkylene(C₁₋₆haloalkyl)NR^{x}R^{y}, 4-6 membered heterocycloalkyl, C(O)OR^{x}, C₀₋₆alkylene-C(O)NR^{x}R^{y}, OC₁₋₃alkylene-heterocycloalkyl, OC₁₋₃alkylene-C(O)NR^{x}R^{y}, O(C₁₋₆alkyl)SO₂NR^{x}NR^{y}, NR^{x}R^{y}, NHSO₂R^{x}, SR^{x}, S—C₁₋₆alkylene-C(O)NR^{x}R^{y}, S(O)R^{x}R^{y}, SO₂R^{x}, SO₂NR^{x}R^{y}, S(O)(NR^{x})R^{y}, S(O)(NR^{x})R^{y}, or SO₂R^{x};
   wherein each R^{x} and R^{y} is independently H, D, C₁₋₆alkyl, C₁₋₆haloalkyl, C₁₋₆alkoxy, C₃₋₆cycloalkyl, C₁₋₆alkylene-amide, OC₀₋₂alkylene-heterocycloalkyl, 4-6 membered heterocycloalkyl, C(O)C₁₋₆alkyl, imino, or C₁₋₆alkylsulfonyl; or R^{x} and R^{y} together with the atoms to which R^{x} and R^{y} are bound can form an optionally substituted ring;
   R⁷ is C₁₋₃alkyl, C₃₋₆cycloalkyl, cyano, or halogen;
   R^{b1}, R^{b2}, and R^{b3} are each independently H or D;
   R⁸ is H, D, or halogen; and
   n is 0, 1, or 2.

In some embodiments, the compound is of Formula (Ia):

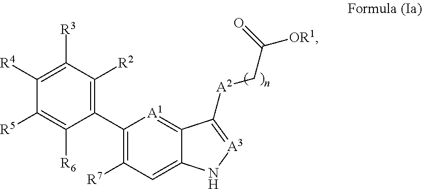

or a pharmaceutically acceptable salt, tautomer, or pharmaceutically acceptable salt of the tautomer thereof.

In some embodiments, A¹ is CR⁸. In some embodiments, A¹ is N. In some embodiments, R⁸ is H. In some embodiments, R⁸ is halogen. In a preferred embodiment, A¹ is CH or CF.

In some embodiments, A² is O or NH. In a preferred embodiment, A² is CH₂ or S. In a preferred embodiment, A² is CH₂. In a preferred embodiment, A² is S.

In some embodiments, A³ is CH. In a preferred embodiment, A³ is N. In a preferred embodiment, A¹ is N and A³ is N.

In some embodiments, R¹ is H, D, or C₁₋₈alkyl. In some embodiments, R¹ is C₃₋₆cycloalkyl or 4-6 membered heterocycloalkyl. In some embodiments, R¹ is -6-O-3,4,5-trihydroxy-tetrahydro-2H-pyran-2-carboxylic acid. In a preferred embodiment, R¹ is H.

In some embodiments, R² is H. In some embodiments, R² is halogen. In some embodiments, R² is F. In some embodiments, R² is Cl. In some embodiments, R³ is H. In some embodiments, R³ is halogen. In some embodiments, R³ is F. In some embodiments, R³ is Cl. In some embodiments, R⁵ is H. In some embodiments, R⁵ is halogen. In some embodiments, R⁵ is F. In some embodiments, R⁵ is Cl. In some embodiments, R⁶ is H. In some embodiments, R⁶ is halogen. In some embodiments, R⁶ is OH. In some embodiments, R⁶ is F. In some embodiments, R⁶ is Cl. In a preferred embodiment, R² is halogen; and R³, R⁵, and R⁶ are each independently H. In a preferred embodiment, R², R³, R⁵, and R⁶ are each independently H.

In some embodiments, R⁴ is 6-membered monocyclic aryl. In some embodiments, R⁴ is 6-membered monocyclic aryl substituted with 1, 2, or 3 Rₐ. In some embodiments, R⁴ is 5 or 6-membered monocyclic heteroaryl substituted with 1, 2, or 3 Rₐ. In a preferred embodiment, R⁴ is phenyl, wherein R⁹, R¹⁰, R¹¹, R¹², and R¹³ are each independently H, D, Cl, F, CN, C₁₋₃alkyl, C₁₋₆alkylene-OH, C₁₋₆alkylene-OC₁₋₆alkyl, OH, OC₁₋₆alkyl, OC₁₋₆haloalkyl, O(C₁₋₃alkylene)heterocycloalkyl, O(C₁₋₃alkylene)-C(O)NR^{x}R^{y}, C₁₋₃alkylene-NR^{x}R^{y}, C(O)OH, C(O)OC₁₋₃alkyl, C₀₋₂alkylene-C(O)NR^{x}R^{y}, SO₂NR^{x}R^{y}, S(O)(NR^{x})R^{y}, NR^{x}R^{y}, SR^{x}, or SO₂R^{x}. In a preferred embodiment, R⁴ is 6-membered monocyclic heteroaryl, wherein at least one of R⁹, R¹⁰, R¹¹, R¹², and R¹³ is C₁₋₃alkoxy, halogen, hydroxy, or C(O)NH₂.

In some embodiments, R⁷ is C₁₋₃alkyl. In some embodiments, R⁷ is C₃₋₆cycloalkyl. In some embodiments, R⁷ is cyclopropyl. In some embodiments, R⁷ is cyano. In some embodiments, R⁷ is halogen. In a preferred embodiment, R⁷ is Cl. In a preferred embodiment, R⁷ is F.

The present disclosure also provides a compound of Formula (II):

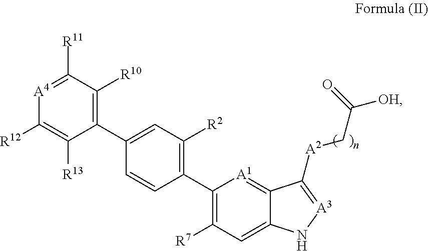

a pharmaceutically acceptable salt, a tautomer, or a pharmaceutically acceptable salt of the tautomer thereof, wherein:
   R² is H, D or halogen;
   R⁷ is Cl or CN;
   A² is CH₂, CHD, CD₂, S, or NH;
   A⁴ is CR⁹ or N;
   R⁹ is H, F, Cl, C₁₋₃alkyl, C₁₋₃alkylene-O—C₁₋₃alkyl, C₁₋₃alkylene-NH₂, COOH, C(O)OC₁₋₃alkyl, C₁₋₃alkylene-C(O)NH₂, C(O)NHC₁₋₃alkyl, C(O)N(C₁₋₃alkyl)₂, C₁₋₆alkylene(C₁₋₆haloalkyl)NH₂, C₀₋₂alkylene-NH(C(O)C₁₋₃alkyl), OC₁₋₃alkyl, OC₁₋₃haloalkyl, OC₁₋₃alkylene-heterocycloalkyl, OC₁₋₃alkylene-C(O)NH₂, NHSO₂C₁₋₃alkyl, N(C₁₋₃ alkyl)(C(O)C₁₋₃alkyl), SC₁₋₃alkyl, S—C₁₋₃alkylene-C(O)NH₂, SO(NH)C₁₋₃alkyl, SO₂NH₂, or SO₂C₁₋₃alkyl;
   R¹⁰ is H, D, or OH;
   R¹¹ is H, D, halogen, CN, O(C₁₋₃alkyl), or O(C₁₋₃haloalkyl); or R⁹ and R¹¹ together with the carbon atom to which R⁹ and R¹¹ are bound form an optionally substituted ring;
   R¹² is H, OC₁₋₃alkyl, or C₁₋₃alkylene-OH;
   R¹³ is H, F, Cl, or C₁₋₃alkyl; and
   n is 1 or 2.

In some embodiments, R⁷ is CN. In a preferred embodiment, R⁷ is Cl.

In some embodiments, R⁹ is H, F, Cl, C₁₋₃alkyl, C₁₋₃alkylene-O—C₁₋₃alkyl, C₁₋₃alkylene-NH₂, COOH, C(O)OC₁₋₃alkyl, C₁₋₃alkylene-C(O)NH₂, C(O)NHC₁₋₃alkyl, C(O)N(C₁₋₃alkyl)₂, C₁₋₃haloalkylene-NH₂, C₀₋₂alkylene-NH(C(O)C₁₋₃alkyl), OC₁₋₃alkyl, OC₁₋₃haloalkyl, OC₁₋₃alkylene-heterocycloalkyl, OC₁₋₃alkylene-C(O)NH₂, NHSO₂C₁₋₃alkyl, N(C₁₋₃alkyl)(C(O)C₁₋₃alkyl), SC₁₋₃ alkyl, S—C₁₋₃alkylene-C(O)NH₂, SO(NH)C₁₋₃alkyl, SO₂NH₂, or SO₂C₁₋₃alkyl. In some embodiments, R⁹ is H, C₁₋₃alkylene-O—C₁₋₃alkyl, C₁₋₃alkylene-NH₂, C₁₋₃alkylene-C(O)NH₂, C₁₋₃alkylene(C₁₋₃haloalkyl)NH₂, or C₀₋₂alkylene-NH(C(O)C₁₋₃alkyl). In some embodiments, R⁹ is NHSO₂C₁₋₃alkyl or N(C₁₋₃alkyl)(C(O)C₁₋₃alkyl). In some embodiments, R⁹ is SC₁₋₃alkyl, S—C₁₋₃ alkylene-C(O)NH₂, SO(NH)C₁₋₃alkyl, SO₂NH₂, or SO₂C₁₋₃alkyl. In a preferred embodiment, R⁹ is H. In a preferred embodiment, R⁹ is C(O)NH₂. In a preferred embodiment, R⁹ is C₁₋₃alkylene-NH₂.

In some embodiments, R¹⁰ is H or D. In a preferred embodiment, R¹⁰ is OH.

In some embodiments, R¹¹ is H, F, Cl, or CN. In some embodiments, R¹¹ is O(C₁₋₃alkyl) or O(C₁₋₃haloalkyl). In a preferred embodiment, R¹¹ is H.

The present disclosure further provides a compound of Formula (III):

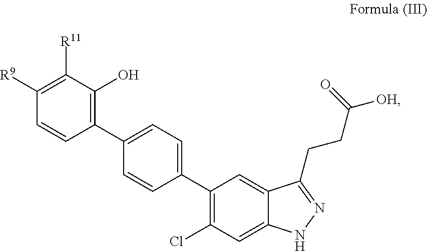

a pharmaceutically acceptable salt, a tautomer, or a pharmaceutically acceptable salt of the tautomer thereof, wherein:
   R⁹ is H, D, halogen, C₁₋₃alkyl, C(O)NH₂, C₁₋₃alkylene-NH₂, C₁₋₃alkylene-O—C₁₋₃alkyl, C(O)OC₁₋₃alkyl, C(O)OH, OC₁₋₃alkyl, OC₁₋₃haloalkyl, —O(C₁₋₃alkyl)SO₂NH₂, C₁₋₆alkylene(C₁₋₆haloalkyl)NH₂, SC₁₋₃alkyl, or —C(O)NR^{x}R^{y}, wherein each R^{x} and R^{y} are independently H or C₁₋₆alkyl; and
   R¹¹ is H, D, halogen, CN, or —O(C₁₋₃alkyl); or R⁹ and R¹¹ together with the carbon atom to which R⁹ and R¹¹ are bound form an optionally substituted ring.

In a preferred embodiment, R⁹ is H or —C(O)NR^{x}R^{y}, wherein each R^{x} and R^{y} are independently H or C₁₋₆alkyl; and R¹¹ is H or —O(C₁₋₃alkyl); or R⁹ and R¹¹ together with the carbon atom to which R⁹ and R¹¹ are bound form an optionally substituted ring.

The present disclosure also provides compounds of Formula (IVa), Formula (IVb), or Formula (IVc):

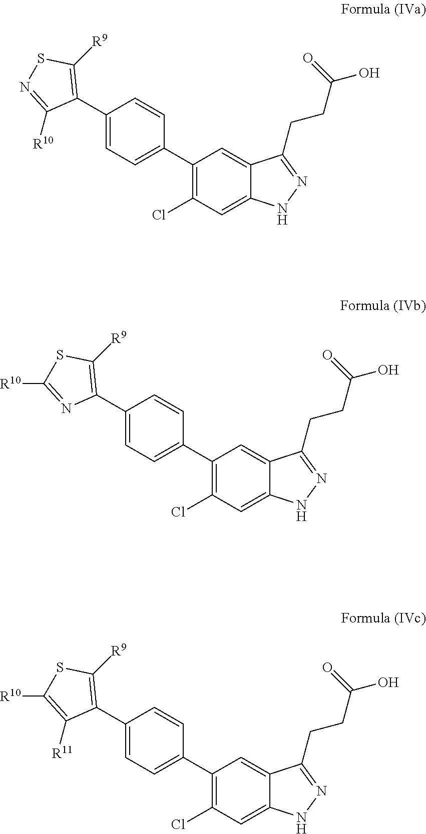

a pharmaceutically acceptable salt, a tautomer, or a pharmaceutically acceptable salt of the tautomer thereof, wherein each R⁹, R¹⁰, and R¹¹ are independently H, C₁₋₃alkyl, C₁₋₃alkylene-OH, or OC₁₋₃alkyl.

In a preferred embodiment, R⁹, R¹⁰, and R¹¹ are each independently H. In a preferred embodiment, R¹⁰ is C₁₋₃alkyl, C₁₋₃alkylene-OH or OC₁₋₃alkyl.

The present disclosure also provides a compound of Formula (V):

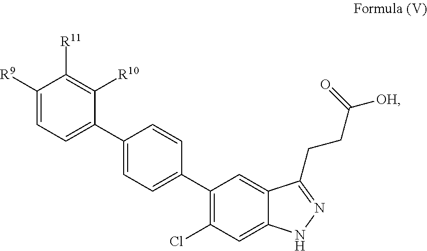

a pharmaceutically acceptable salt, a tautomer, or a pharmaceutically acceptable salt of the tautomer thereof, wherein:
   R¹⁰ is H, D, or OH;
   R⁹ and R¹¹ together with the carbon atom to which R⁹ and R¹¹ are bound form an optionally substituted ring, wherein the ring is 5- or 6-membered heterocycloalkyl or 5- or 6-membered heteroaryl, wherein the ring is optionally substituted with C₁₋₃alkyl, OH, or oxo.

In some embodiments, the ring is 6-membered heterocycloalkyl. In a preferred embodiment, the ring is 5-membered heterocycloalkyl. In some embodiments, the ring is 6-membered heteroaryl. In a preferred embodiment, the ring is 5-membered heteroaryl.

In a preferred embodiment, the ring is substituted with C₁₋₃alkyl. In a preferred embodiment, the ring is substituted with OH. In a preferred embodiment, the ring is substituted with oxo.

In some embodiments, the compound of the disclosure, a pharmaceutically acceptable salt, a tautomer, or a pharmaceutically acceptable salt of the tautomer thereof, is selected from the group consisting of:
3-(6-Chloro-5-(2′-hydroxy-[1,1′-biphenyl]-4-yl)-1H-indazol-3-yl)propanoic acid;
3-(6-chloro-5-(2′-hydroxy-6′-methyl-[1,1′-biphenyl]-4-yl)-1H-indazol-3-yl)propanoic acid;
3-(6-chloro-5-(2′-hydroxy-3′-methoxy-[1,1′-biphenyl]-4-yl)-1H-indazol-3-yl)propanoic acid;
3-(6-chloro-5-(2′-hydroxy-3′-methoxy-6′-methyl-[1,1′-biphenyl]-4-yl)-1H-indazol-3-yl)propanoic acid;
3-(6-chloro-5-(4-(7-hydroxy-2,3-dihydrobenzofuran-6-yl)phenyl)-1H-indazol-3-yl)propanoic acid;
3-(6-chloro-5-(2′-hydroxy-4′-(methoxymethyl)-[1,1′-biphenyl]-4-yl)-1H-indazol-3-yl)propanoic acid;
3-(6-chloro-5-(2′-hydroxy-4′,6′-dimethyl-[1,1′-biphenyl]-4-yl)-1H-indazol-3-yl)propanoic acid;
3-(6-chloro-5-(3′-fluoro-2′-hydroxy-6′-methyl-[1,1′-biphenyl]-4-yl)-1H-indazol-3-yl)propanoic acid;
3-(6-chloro-5-(4′-fluoro-2′-hydroxy-3′-methoxy-[1,1′-biphenyl]-4-yl)-1H-indazol-3-yl)propanoic acid;
3-(6-chloro-5-(4′-(dimethylcarbamoyl)-[1,1′-biphenyl]-4-yl)-1H-indazol-3-yl)propanoic acid;
4-(6-chloro-5-(2′-hydroxy-3′-methoxy-[1,1′-biphenyl]-4-yl)-1H-indazol-3-yl)butanoic acid;
3-(6-chloro-5-(4′-(methylcarbamoyl)-[1,1′-biphenyl]-4-yl)-1H-indazol-3-yl)propanoic acid;
6-((3-(6-chloro-5-(2′-hydroxy-[1,1′-biphenyl]-4-yl)-1H-indazol-3-yl)propanoyl)oxy)-3,4,5-trihydroxytetrahydro-2H-pyran-2-carboxylic acid; and
6-((4′-(3-(2-carboxyethyl)-6-chloro-1H-indazol-5-yl)-[1,1′-biphenyl]-2-yl)oxy)-3,4,5-trihydroxytetrahydro-2H-pyran-2-carboxylic acid.

In a preferred embodiment, the compound is 3-[6-chloro-5-(2′-hydroxy[1,1′-biphenyl]-4-yl)-1H-indazol-3-yl]propanoic acid, a pharmaceutically acceptable salt, a tautomer, or a pharmaceutically acceptable salt of the tautomer thereof. In a preferred embodiment, the compound is 3-[6-chloro-5-(2′-hydroxy[1,1′-biphenyl]-4-yl)-1H-indazol-3-yl]propanoic acid. In a preferred embodiment, the compound has the structure:

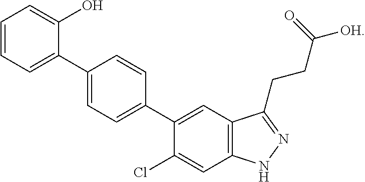 [SMILES: O=C(O)CCc1n[nH]c2cc(Cl)c(-c3ccc(-c4ccccc4O)cc3)cc12]

Any compound herein can be purified. A compound herein can be least 1% pure, at least 2% pure, at least 3% pure, at least 4% pure, at least 5% pure, at least 6% pure, at least 7% pure, at least 8% pure, at least 9% pure, at least 10% pure, at least 11% pure, at least 12% pure, at least 13% pure, at least 14% pure, at least 15% pure, at least 16% pure, at least 17% pure, at least 18% pure, at least 19% pure, at least 20% pure, at least 21% pure, at least 22% pure, at least 23% pure, at least 24% pure, at least 25% pure, at least 26% pure, at least 27% pure, at least 28% pure, at least 29% pure, at least 30% pure, at least 31% pure, at least 32% pure, at least 33% pure, at least 34% pure, at least 35% pure, at least 36% pure, at least 37% pure, at least 38% pure, at least 39% pure, at least 40% pure, at least 41% pure, at least 42% pure, at least 43% pure, at least 44% pure, at least 45% pure, at least 46% pure, at least 47% pure, at least 48% pure, at least 49% pure, at least 50% pure, at least 51% pure, at least 52% pure, at least 53% pure, at least 54% pure, at least 55% pure, at least 56% pure, at least 57% pure, at least 58% pure, at least 59% pure, at least 60% pure, at least 61% pure, at least 62% pure, at least 63% pure, at least 64% pure, at least 65% pure, at least 66% pure, at least 67% pure, at least 68% pure, at least 69% pure, at least 70% pure, at least 71% pure, at least 72% pure, at least 73% pure, at least 74% pure, at least 75% pure, at least 76% pure, at least 77% pure, at least 78% pure, at least 79% pure, at least 80% pure, at least 81% pure, at least 82% pure, at least 83% pure, at least 84% pure, at least 85% pure, at least 86% pure, at least 87% pure, at least 88% pure, at least 89% pure, at least 90% pure, at least 91% pure, at least 92% pure, at least 93% pure, at least 94% pure, at least 95% pure, at least 96% pure, at least 97% pure, at least 98% pure, at least 99% pure, at least 99.1% pure, at least 99.2% pure, at least 99.3% pure, at least 99.4% pure, at least 99.5% pure, at least 99.6% pure, at least 99.7% pure, at least 99.8% pure, or at least 99.9% pure.

### Pharmaceutically Acceptable Salts

Salts encompassed within the term “pharmaceutically acceptable salts” refer to the compounds of this invention which are generally prepared by reacting the free base or free acid with a suitable organic or inorganic acid, or a suitable organic or inorganic base, respectively, to provide a salt of the compound of the invention that is suitable for administration to a subject or patient. For a review on suitable salts, see Paulekun, G. S. et al., Trends in Active Pharmaceutical Ingredient Salt Selection Based on Analysis of the Orange Book Database, J. Med. Chem. 2007; 50(26), 6665-6672.

In addition, the compounds of the disclosure may also include other salts of such compounds which are not necessarily pharmaceutically acceptable salts, which may be useful as intermediates for one or more of the following: 1) preparing compounds of Formula I, Ia, II, III, IVa-c, and V; 2) purifying compounds of Formula I, Ia, II, III, IVa-c, and V; 3) separating enantiomers of compounds of Formula I, Ia, II, III, IVa-c, and V; or 4) separating diastereomers of compounds of Formula I, Ia, II, III, IVa-c, and V.

Suitable acid addition salts are formed from acids which form non-toxic salts. Examples include, but are not limited to, acetate, adipate, aspartate, benzoate, besylate, bicarbonate/carbonate, bisulfate/sulfate, borate, camsylate, citrate, cyclamate, edisylate, esylate, formate, fumarate, gluceptate, gluconate, glucuronate, hexafluorophosphate, hibenzate, hydrochloride/chloride, hydrobromide/bromide, hydroiodide/iodide, isethionate, lactate, malate, maleate, malonate, mesylate, methylsulfate, naphthylate, 2-napsylate, nicotinate, nitrate, orotate, oxalate, palmitate, pamoate, phosphate/hydrogen phosphate/dihydrogen phosphate, pyroglutamate, saccharate, stearate, succinate, tannate, tartrate, tosylate, trifluoroacetate, 1,5-naphathalenedisulfonic acid and xinafoate salts.

Suitable base salts are formed from bases which form non-toxic salts. Examples include, but are not limited to aluminum, arginine, benzathine, calcium, choline, diethylamine, diolamine, glycine, lysine, magnesium, meglumine, olamine, potassium, sodium, tromethamine and zinc salts.

Hemisalts of acids and bases may also be formed, for example, hemisulfate and hemicalcium salts.

Pharmaceutically acceptable salts of compounds of the invention may be prepared by methods well known to one skilled in the art, including but not limited to the following procedures:
(i) by reacting a compound of the invention with the desired acid or base;
(ii) by removing an acid- or base-labile protecting group from a suitable precursor of a compound of the invention or by ring-opening a suitable cyclic precursor, for example, a lactone or lactam, using the desired acid or base; or
(iii) by converting one salt of a compound of the invention to another. This may be accomplished by reaction with an appropriate acid or base or by means of a suitable ion exchange procedure.

These procedures are typically carried out in solution. The resulting salt may precipitate out and be collected by filtration or may be recovered by evaporation of the solvent.

### Solvates

The compounds of the invention, and pharmaceutically acceptable salts thereof, may exist in unsolvated and solvated forms. The term ‘solvate’ is used herein to describe a molecular complex comprising the compound of the invention, a pharmaceutically acceptable salt thereof, a tautomer thereof, or a pharmaceutically acceptable salt of the tautomer thereof, and one or more pharmaceutically acceptable solvent molecules, for example, ethanol. The term ‘hydrate’ is employed when said solvent is water.

In addition, the compounds of Formula I, Ia, II, III, IVa-c, and V may also include other solvates of such compounds which are not necessarily pharmaceutically acceptable solvates, which may be useful as intermediates for one or more of the following: 1) preparing compounds of Formula I, Ia, II, III, IVa-c, and V; 2) purifying compounds of Formula I, Ia, II, III, IVa-c, and V; 3) separating enantiomers of compounds of Formula I, Ia, II, III, IVa-c, and V; or 4) separating diastereomers of compounds of Formula I, Ia, II, III, IVa-c, and V.

A currently accepted classification system for organic hydrates is one that defines isolated site, channel, or metal-ion coordinated hydrates—see Polymorphism in Pharmaceutical Solids by K. R. Morris (Ed. H. G. Brittain, Marcel Dekker, 1995). Isolated site hydrates are ones in which the water molecules are isolated from direct contact with each other by intervening organic molecules. In channel hydrates, the water molecules lie in lattice channels where they are next to other water molecules. In metal-ion coordinated hydrates, the water molecules are bonded to the metal ion.

When the solvent or water is tightly bound, the complex may have a well-defined stoichiometry independent of humidity. When, however, the solvent or water is weakly bound, as in channel solvates and hygroscopic compounds, the water/solvent content may be dependent on humidity and drying conditions. In such cases, non-stoichiometry will be the norm.

### Complexes

Also included within the scope of the invention are multi-component complexes (other than salts and solvates) wherein the drug and at least one other component are present in stoichiometric or non-stoichiometric amounts. Complexes of this type include clathrates (drug-host inclusion complexes) and co-crystals. The latter are typically defined as crystalline complexes of neutral molecular constituents which are bound together through non-covalent interactions, for example, hydrogen bonded complex (cocrystal) may be formed with either a neutral molecule or with a salt. Co-crystals may be prepared by melt crystallization, by recrystallization from solvents, or by physically grinding the components together—see Chem Commun, 17; 1889-1896, by O. Almarsson and M. J. Zaworotko (2004). For a general review of multi-component complexes, see J Pharm Sci, 64(8), 1269-1288, by Haleblian (August 1975).

### Solid Form

The compounds of the invention may exist in a continuum of solid states ranging from fully amorphous to fully crystalline. The term ‘amorphous’ refers to a state in which the material lacks long range order at the molecular level and, depending upon temperature, may exhibit the physical properties of a solid or a liquid. Typically, such materials do not give distinctive X-ray diffraction patterns and, while exhibiting the properties of a solid, are more formally described as a liquid. Upon heating, a change from solid to liquid properties occurs which is characterized by a change of state, typically second order (‘glass transition’). The term ‘crystalline’ refers to a solid phase in which the material has a regular ordered internal structure at the molecular level and gives a distinctive X-ray diffraction pattern with defined peaks. Such materials when heated sufficiently will also exhibit the properties of a liquid, but the change from solid to liquid is characterized by a phase change, typically first order (‘melting point’).

The compounds of the invention may also exist in a mesomorphic state (mesophase or liquid crystal) when subjected to suitable conditions. The mesomorphic state is intermediate between the true crystalline state and the true liquid state (either melt or solution) and consists of two dimensional order on the molecular level. Mesomorphism arising as the result of a change in temperature is described as ‘thermotropic’ and that resulting from the addition of a second component, such as water or another solvent, is described as ‘lyotropic’. Compounds that have the potential to form lyotropic mesophases are described as ‘amphiphilic’ and consist of molecules which possess an ionic (such as —COO⁻Na⁺, —COO⁻K⁺, or —SO₃⁻Na⁺) or non-ionic (such as —N⁻N⁺(CH₃)₃) polar head group. For more information, see Crystals and the Polarizing Microscope by N. H. Hartshorne and A. Stuart, 4^{th} Edition (Edward Arnold, 1970).

In a preferred embodiment, a compound of the disclosure is a crystalline of the compound. In a preferred embodiment, the compound is crystalline 3-[6-Chloro-5-(2′-hydroxy[1,1′-biphenyl]-4-yl)-1H-indazol-3-yl]propanoic acid, a pharmaceutically acceptable salt, a tautomer, or a pharmaceutically acceptable salt of the tautomer thereof. In a preferred embodiment, the compound is crystalline 3-[6-Chloro-5-(2′-hydroxy[1,1′-biphenyl]-4-yl)-1H-indazol-3-yl]propanoic acid, a pharmaceutically acceptable salt, a tautomer, or a pharmaceutically acceptable salt of the tautomer thereof, having an X-ray powder diffraction pattern comprising diffraction peaks of about 12.6±0.2, about 18.8±0.2, about 19.7±0.2, and about 24.4±0.2 degrees two theta.

### Stereoisomers

Compounds of the invention may exist as two or more stereoisomers. Stereoisomers of the compounds may include cis and trans isomers (geometric isomers), optical isomers such as R and S enantiomers, diastereomers, rotational isomers, atropisomers, and conformational isomers. For example, compounds of the invention containing one or more asymmetric carbon atoms may exist as two or more stereoisomers. Cis/trans isomers may also exist for saturated rings. Cis/trans isomers may be separated by conventional techniques well known to those skilled in the art, for example, chromatography and fractional crystallization.

The pharmaceutically acceptable salts, tautomers, or pharmaceutically acceptable salts of the tautomers of compounds of the invention may also contain a counterion which is optically active (e.g., d-lactate or I-lysine) or racemic (e.g. dl-tartrate or dl-arginine).

Conventional techniques for the preparation/isolation of individual enantiomers include chiral synthesis from a suitable optically pure precursor or resolution of the racemate (or the racemate of a salt or derivative) using, for example, chiral high pressure liquid chromatography (HPLC). Alternatively, the racemate (or a racemic precursor) may be reacted with a suitable optically active compound, for example, an alcohol, or, in the case where a compound of the invention contains an acidic or basic moiety, a base or acid such as 1-phenylethylamine or tartaric acid. The resulting diastereomeric mixture may be separated by chromatography, fractional crystallization, or by using both of said techniques, and one or both of the diastereoisomers converted to the corresponding pure enantiomer(s) by means well known to a skilled person. Chiral compounds of the invention (and chiral precursors thereof) may be obtained in enantiomerically-enriched form using chromatography, typically HPLC Concentration of the eluate affords the enriched mixture. Chiral chromatography using sub- and supercritical fluids may be employed. Methods for chiral chromatography useful in some embodiments of the present invention are known in the art (see, for example, Smith, Roger M., Loughborough University, Loughborough, UK; Chromatographic Science Series (1998), 75 (Supercritical Fluid Chromatography with Packed Columns), pp. 223-249 and references cited therein).

When any racemate crystallizes, crystals of two different types are possible. The first type is the racemic compound (true racemate) referred to above wherein one homogeneous form of crystal is produced containing both enantiomers in equimolar amounts. The second type is the racemic mixture or conglomerate wherein two crystal forms are produced in equimolar amounts each comprising a single enantiomer. While both of the crystal forms present in a racemic mixture have identical physical properties, they may have different physical properties compared to the true racemate. Racemic mixtures may be separated by conventional techniques known to those skilled in the art—see, for example, Stereochemistry of Organic Compounds by E. L. Eliel and S. H. Wilen (Wiley, 1994).

### Tautomerism

Where structural isomers are interconvertible via a low energy barrier, tautomeric isomerism (‘tautomerism’) may occur. This may take the form of proton tautomerism in compounds of the invention containing, for example, an imino/amino, keto/enol, or oxime/nitroso group, lactam/lactim or so-called valence tautomerism in compounds which contain an aromatic moiety. It follows that a single compound may exhibit more than one type of isomerism.

It must be emphasized that while, for conciseness, the compounds of the invention have been drawn herein in a single tautomeric form, all possible tautomeric forms are included within the scope of the invention.

### Isotopes

The present invention includes all pharmaceutically acceptable isotopically-labeled compounds of the invention wherein one or more atoms are replaced by atoms having the same atomic number, but an atomic mass or mass number different from the atomic mass or mass number which predominates in nature.

Examples of isotopes suitable for inclusion in the compounds of the invention may include isotopes of hydrogen, such as ²H (D, deuterium) and ³H (T, tritium), carbon, such as ¹¹C, ¹³C and ¹⁴C, chlorine, such as ³⁶Cl, fluorine, such as ¹⁸F, iodine, such as ¹²³I and ¹²⁵I nitrogen, such as ¹³N and ¹⁵N, oxygen, such as ¹⁵O, ¹⁷O and ¹⁸O, phosphorus, such as ³²P, and sulfur, such as ³⁵S.

Certain isotopically-labelled compounds of the invention, for example those incorporating a radioactive isotope, are useful in one or both of drug or substrate tissue distribution studies. The radioactive isotopes, such as, tritium and ¹⁴C are particularly useful for this purpose in view of their ease of incorporation and ready means of detection. Substitution with positron emitting isotopes, such as, ¹¹C ¹⁸F, ¹⁵O and ¹³N, may be useful in Positron Emission Topography (PET) studies for examining substrate receptor occupancy. Substitution with deuterium may afford certain therapeutic advantages resulting from greater metabolic stability, for example increased in vivo half-life, reduced dosage requirements, reduced CYP450 inhibition (competitive or time dependent), or an improvement in therapeutic index or tolerability.

In some embodiments, the disclosure provides deuterium-labeled (or deuterated) compounds and salts, where the formula and variables of such compounds and salts are each and independently as described herein. “Deuterated” means that at least one of the atoms in the compound is deuterium in an abundance that is greater than the natural abundance of deuterium (typically approximately 0.015%). A skilled artisan recognized that in chemical compounds with a hydrogen atom, the hydrogen atom actually represents a mixture of H and D, with about 0.015% being D. The concentration of the deuterium incorporated into the deuterium-labeled compounds and salt of the invention may be defined by the deuterium enrichment factor. It is understood that one or more deuterium may exchange with hydrogen under physiological conditions.

In some embodiments, the deuterium compound is selected from any one of the compounds set forth in Table 5 shown in the Examples section. In some embodiments, one or more hydrogen atoms on certain metabolic sites on the compounds of the invention are deuterated. In some embodiments, the deuterium compound is selected from the group consisting of:

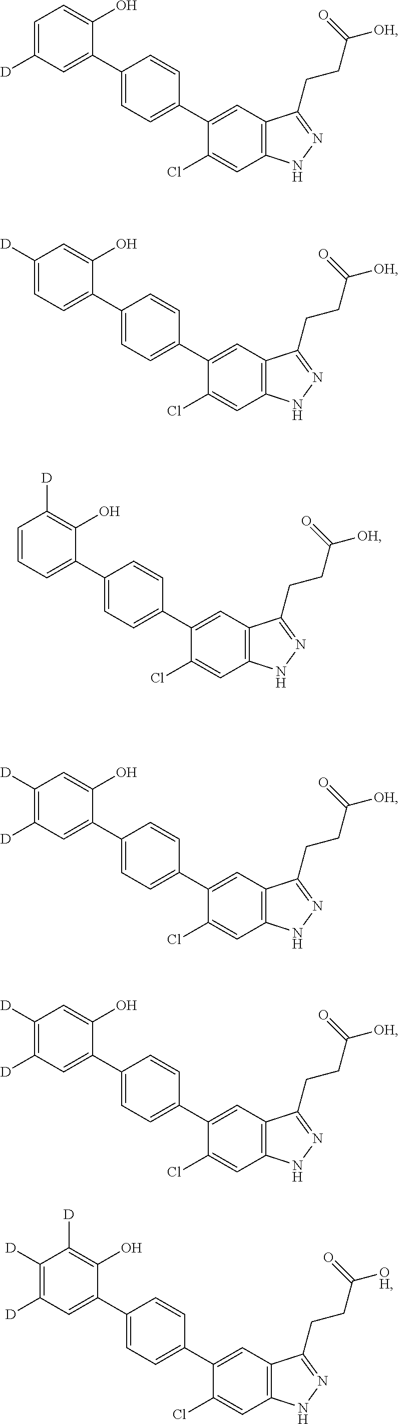 [SMILES: [2H]c1ccc(O)c(-c2ccc(-c3cc4c(CCC(=O)O)n[nH]c4cc3Cl)cc2)c1 ; [2H]c1ccc(-c2ccc(-c3cc4c(CCC(=O)O)n[nH]c4cc3Cl)cc2)c(O)c1 ; [2H]c1cccc(-c2ccc(-c3cc4c(CCC(=O)O)n[nH]c4cc3Cl)cc2)c1O ; [2H]c1cc(O)c(-c2ccc(-c3cc4c(CCC(=O)O)n[nH]c4cc3Cl)cc2)cc1[2H] ; [2H]c1cc(O)c(-c2ccc(-c3cc4c(CCC(=O)O)n[nH]c4cc3Cl)cc2)cc1[2H] ; [2H]c1cc(-c2ccc(-c3cc4c(CCC(=O)O)n[nH]c4cc3Cl)cc2)c(O)c([2H])c1[2H]]

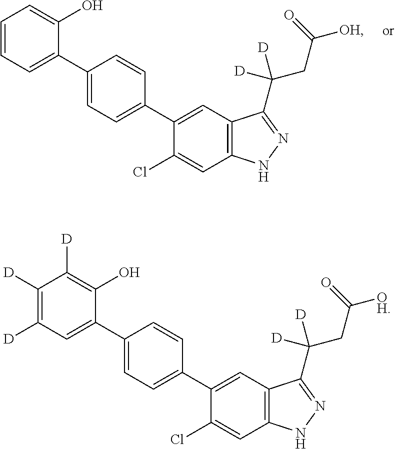 [SMILES: [2H]C([2H])(CC(=O)O)c1n[nH]c2cc(Cl)c(-c3ccc(-c4ccccc4O)cc3)cc12 ; [2H]c1cc(-c2ccc(-c3cc4c(C([2H])([2H])CC(=O)O)n[nH]c4cc3Cl)cc2)c(O)c([2H])c1[2H]]

Isotopically-labeled compounds of the invention may generally be prepared by conventional techniques known to those skilled in the art or by processes analogous to those described in the accompanying Examples and Preparations using an appropriate isotopically-labeled reagent in place of the non-labeled reagent previously employed.

Pharmaceutically acceptable solvates in accordance with the invention include those wherein the solvent of crystallization may be isotopically substituted, e.g., D₂O, d₆-acetone, d₆-DMSO.

### Prodrugs

A compound of the invention may be administered in the form of a prodrug. Thus, certain derivatives of a compound of the invention which may have little or no pharmacological activity themselves may, when administered into or onto the body, be converted into a compound of the invention having the desired activity, for example by hydrolytic cleavage, particularly hydrolytic cleavage promoted by an esterase or peptidase enzyme. Such derivatives are referred to as ‘prodrugs’. Further information on the use of prodrugs may be found in ‘The Expanding Role of Prodrugs in Contemporary Drug Design and Development, Nature Reviews Drug Discovery, 17, 559-587 (2018) (J. Rautio et al.).

Prodrugs in accordance with the invention may, for example, be produced by replacing appropriate functionalities present in compounds of the invention with certain moieties known to those skilled in the art as ‘pro-moieties’ as described, for example, in ‘Design of Prodrugs’ by H. Bundgaard (Elsevier, 1985).

Thus, a prodrug in accordance with the invention may be (a) an ester or amide derivative of a carboxylic acid when present in a compound of the invention; (b) an ester, carbonate, carbamate, phosphate or ether derivative of a hydroxyl group when present in a compound of the invention; (c) an amide, imine, carbamate or amine derivative of an amino group when present in a compound of the invention; (d) a thioester, thiocarbonate, thiocarbamate or sulfide derivatives of a thiol group when present in a compound of the invention; or an oxime or imine derivative of a carbonyl group when present in a compound of the invention.

Some specific examples of prodrugs in accordance with the invention include:
(I) when a compound of the invention contains a carboxylic acid functionality (—COOH), an ester thereof, such as a compound wherein the hydrogen of the carboxylic acid functionality of the compound is replaced by C₁-C₈ alkyl (e.g., ethyl) or (C₁-C₈ alkyl)C(═O)OCH₂— (e.g., ^{t}BuC(═O)OCH₂—);
(ii) when a compound of the invention contains an alcohol functionality (—OH), an ester thereof, such as a compound wherein the hydrogen of the alcohol functionality of the compound is replaced by —CO(C₁-C₈ alkyl) (e.g., methylcarbonyl) or the alcohol is esterified with an amino acid;
(iii) when a compound of the invention contains an alcohol functionality (—OH), an ether thereof, such as a compound wherein the hydrogen of the alcohol functionality of the compound is replaced by (C₁-C₈ alkyl)C(═O)OCH₂— or —CH₂OP(═O)(OH)₂;
(iv) when a compound of the invention contains an alcohol functionality (—OH), a phosphate thereof, such as a compound wherein the hydrogen of the alcohol functionality of the compound is replaced by —P(═O)(OH)₂ or —P(═O)(O⁻Na⁺)₂ or —P(═O)(O⁻)₂Ca²⁺;
(v) when a compound of the invention contains a primary or secondary amino functionality (—NH₂ or —NHR where R H), an amide thereof, for example, a compound wherein, as the case may be, one or both hydrogens of the amino functionality of the compound is/are replaced by (C₁-C₁₀)alkanoyl, —COCH₂NH₂ or the amino group is derivatized with an amino acid;
(vi) when a compound of the invention contains a primary or secondary amino functionality (—NH₂ or —NHR where R H), an amine thereof, for example, a compound wherein, as the case may be, one or both hydrogens of the amino functionality of the compound is/are replaced by —CH₂OP(═O)(OH)₂.

Certain compounds of the invention may themselves act as prodrugs of other compounds the invention It is also possible for two compounds of the invention to be joined together in the form of a prodrug. In certain circumstances, a prodrug of a compound of the invention may be created by internally linking two functional groups in a compound of the invention, for instance by forming a lactone.

### Metabolites

Also included within the scope of the invention are active metabolites of compounds of the invention, that is, compounds formed in vivo upon administration of the drug, often by oxidation or dealkylation. Some examples of metabolites in accordance with the invention include, but are not limited to,
(I) where the compound of the invention contains an alkyl group, a hydroxyalkyl derivative thereof (—CH>—COH):
(ii) where the compound of the invention contains an alkoxy group, a hydroxy derivative thereof (—OR->—OH);
(iii) where the compound of the invention contains a tertiary amino group, a secondary amino derivative thereof (—NRR′—>—NHR or —NHR′);
(iv) where the compound of the invention contains a secondary amino group, a primary derivative thereof (—NHR—>—NH₂);
(v) where the compound of the invention contains a phenyl moiety, a phenol derivative thereof (-Ph->-PhOH);
(vi) where the compound of the invention contains an amide group, a carboxylic acid derivative thereof (—CONH₂—>COOH); and
(vii) where the compound contains a hydroxy or carboxylic acid group, the compound may be metabolized by conjugation, for example with glucuronic acid to form a glucuronide. Other routes of conjugative metabolism exist. These pathways are frequently known as Phase 2 metabolism and include, for example, sulfation or acetylation. Other functional groups, such as NH groups, may also be subject to conjugation.

In a preferred embodiment, a metabolite of a compound disclosed herein can comprise an O-3,4,5-trihydroxytetrahydro-2H-pyran-2-carboxylic acid moiety. In a preferred embodiment, the metabolite is 6-((3-(6-chloro-5-(2′-hydroxy-[1,1′-biphenyl]-4-yl)-1H-indazol-3-yl)propanoyl)oxy)-3,4,5-trihydroxytetrahydro-2H-pyran-2-carboxylic acid, a pharmaceutically acceptable salt, a tautomer, or a pharmaceutically acceptable salt of the tautomer thereof. In a preferred embodiment, the metabolite has the structure:

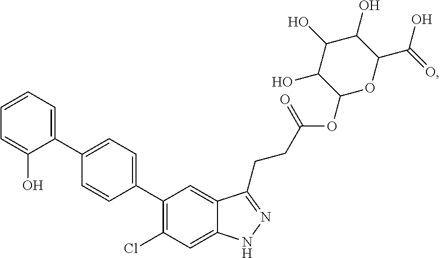 [SMILES: O=C(CCc1n[nH]c2cc(Cl)c(-c3ccc(-c4ccccc4O)cc3)cc12)OC1OC(C(=O)O)C(O)C(O)C1O]

a pharmaceutically acceptable salt, a tautomer, or a pharmaceutically acceptable salt of the tautomer thereof.

In a preferred embodiment, the metabolite is 6-((4′-(3-(2-carboxyethyl)-6-chloro-1H-indazol-5-yl)-[1,1′-biphenyl]-2-yl)oxy)-3,4,5-trihydroxytetrahydro-2H-pyran-2-carboxylic acid, a pharmaceutically acceptable salt, a tautomer, or a pharmaceutically acceptable salt of the tautomer thereof. In a preferred embodiment, the metabolite has the structure:

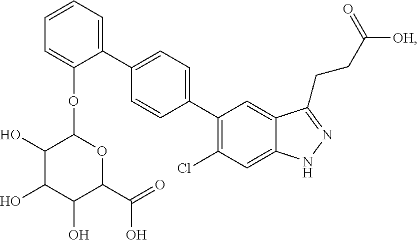 [SMILES: O=C(O)CCc1n[nH]c2cc(Cl)c(-c3ccc(-c4ccccc4OC4OC(C(=O)O)C(O)C(O)C4O)cc3)cc12]

a pharmaceutically acceptable salt, a tautomer, or a pharmaceutically acceptable salt of the tautomer thereof.

In a preferred embodiment, the metabolite is 3-(6-chloro-5-(2′-(sulfooxy)-[1,1′-biphenyl]-4-yl)-1H-indazol-3-yl)propanoic acid, a pharmaceutically acceptable salt, a tautomer, or a pharmaceutically acceptable salt of the tautomer thereof. In some embodiments, the metabolite is 3-(6-chloro-5-(2′-(sulfooxy)-[1,1′-biphenyl]-4-yl)-1H-indazol-3-yl)propanoic acid further comprising a hydroxyl group, or a pharmaceutically acceptable salt, a tautomer, or a pharmaceutically acceptable salt of the tautomer thereof. In some embodiments, the metabolite has the structure:

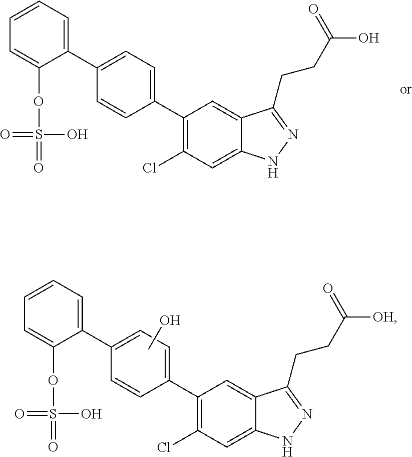

a pharmaceutically acceptable salt, a tautomer, or a pharmaceutically acceptable salt of the tautomer thereof.

### Pharmaceutical Compositions

In another embodiment, the invention comprises pharmaceutical compositions. For pharmaceutical composition purposes, the compound per se, pharmaceutically acceptable salts, tautomers, or pharmaceutically acceptable salts of the tautomers thereof will simply be referred to as the compounds of the invention. A “pharmaceutical composition” refers to a mixture of one or more of the compounds of the invention, or a pharmaceutically acceptable salt, tautomer, solvate, hydrate or prodrug thereof as an active ingredient, and at least one pharmaceutically acceptable excipient.

The term “excipient” is used herein to describe any ingredient other than the compound(s) of the invention. The choice of excipient will to a large extent depend on factors such as the mode of administration, the effect of the excipient on solubility and stability, and the nature of the dosage form.

As used herein, “excipient” includes any and all solvents, dispersion media, coatings, antibacterial and antifungal agents, isotonic and absorption delaying agents, carriers, diluents and the like that are physiologically compatible. Examples of excipients include one or more of water, saline, phosphate buffered saline, dextrose, glycerol, ethanol and the like, as well as combinations thereof, and may include isotonic agents, for example, sugars, sodium chloride, or polyalcohols such as mannitol, or sorbitol in the composition. Examples of excipients also include various organic solvents (such as hydrates and solvates). The pharmaceutical compositions may, if desired, contain additional excipients such as flavorings, binders/binding agents, lubricating agents, disintegrants, sweetening or flavoring agents, coloring matters or dyes, and the like. For example, for oral administration, tablets containing various excipients, such as citric acid may be employed together with various disintegrants such as starch, alginic acid and certain complex silicates and with binding agents such as sucrose, gelatin and acacia. Examples, without limitation, of excipients include calcium carbonate, calcium phosphate, various sugars and types of starch, cellulose derivatives, gelatin, vegetable oils and polyethylene glycols. Additionally, lubricating agents such as magnesium stearate, sodium lauryl sulfate and talc are often useful for tableting purposes. Solid compositions of a similar type may also be employed in soft and hard filled gelatin capsules. Non-limiting examples of excipients, therefore, also include lactose or milk sugar and high molecular weight polyethylene glycols. When aqueous suspensions or elixirs are desired for oral administration the active compound therein may be combined with various sweetening or flavoring agents, coloring matters or dyes and, if desired, emulsifying agents or suspending agents, together with additional excipients such as water, ethanol, propylene glycol, glycerin, or combinations thereof.

Examples of excipients also include pharmaceutically acceptable substances such as wetting agents or minor amounts of auxiliary substances such as wetting or emulsifying agents, preservatives, or buffers, which enhance the shelf life or effectiveness of the compound.

The compositions of this invention may be in a variety of forms. These include, for example, liquid, semi-solid and solid dosage forms, such as liquid solutions (e.g., injectable and infusible solutions), dispersions or suspensions, tablets, capsules, pills, powders, liposomes and suppositories. The form depends on the intended mode of administration and therapeutic application.

Typical compositions are in the form of injectable or infusible solutions, such as compositions similar to those used for passive immunization of humans with antibodies in general. One mode of administration is parenteral (e.g., intravenous, subcutaneous, intraperitoneal, intramuscular). In another embodiment, the compound is administered by intravenous infusion or injection. In yet another embodiment, the compound is administered by intramuscular or subcutaneous injection.

Oral administration of a solid dosage form may be, for example, presented in discrete units, such as hard or soft capsules, pills, cachets, lozenges, or tablets, each containing a predetermined amount of at least one compound of the invention. In another embodiment, the oral administration may be in a powder or granule form. In another embodiment, the oral dosage form is sub-lingual, such as, for example, a lozenge. In such solid dosage forms, the compounds of the invention are ordinarily combined with one or more adjuvants. Such capsules or tablets may comprise a controlled release formulation. In the case of capsules, tablets, and pills, the dosage forms also may comprise buffering agents or may be prepared with enteric coatings. In a preferred embodiment, a compound of the disclosure is administered orally. In a preferred embodiment, a compound of the disclosure is administered orally as a soft capsule, pill, or tablet. In a preferred embodiment, a compound of the disclosure is administered orally in immediate release form. In a preferred embodiment, a compound of the disclosure is administered orally in extended release form.

In another embodiment, oral administration may be in a liquid dosage form. Liquid dosage forms for oral administration include, for example, pharmaceutically acceptable emulsions, solutions, suspensions, syrups, and elixirs containing inert diluents commonly used in the art (e.g., water). Such compositions also may comprise adjuvants, such as one or more of wetting, emulsifying, suspending, flavoring (e.g., sweetening), or perfuming agents.

In another embodiment, the invention comprises a parenteral dosage form. “Parenteral administration” includes, for example, subcutaneous injections, intravenous injections, intraperitoneally, intramuscular injections, intrasternal injections, and infusion. Injectable preparations (i.e., sterile injectable aqueous or oleaginous suspensions) may be formulated according to the known art using one or more of suitable dispersing, wetting agents, or suspending agents.

In another embodiment, the invention comprises a topical dosage form. “Topical administration” includes, for example, dermal and transdermal administration, such as via transdermal patches or iontophoresis devices, intraocular administration, or intranasal or inhalation administration. Compositions for topical administration also include, for example, topical gels, sprays, ointments, and creams. A topical formulation may include a compound which enhances absorption or penetration of the active ingredient through the skin or other affected areas. When the compounds of this invention are administered by a transdermal device, administration will be accomplished using a patch either of the reservoir and porous membrane type or of a solid matrix variety. Typical formulations for this purpose include gels, hydrogels, lotions, solutions, creams, ointments, dusting powders, dressings, foams, films, skin patches, wafers, implants, sponges, fibers, bandages and microemulsions. Liposomes may also be used. Typical excipients include alcohol, water, mineral oil, liquid petrolatum, white petrolatum, glycerin, polyethylene glycol and propylene glycol. Penetration enhancers may be incorporated—see, for example, B. C. Finnin and T. M. Morgan, J. Pharm. Sci., vol. 88, pp. 955-958, 1999.

Formulations suitable for topical administration to the eye include, for example, eye drops wherein the compound of this invention is dissolved or suspended in a suitable excipient. A typical formulation suitable for ocular or aural administration may be in the form of drops of a micronized suspension or solution in isotonic, Ph-adjusted, sterile saline. Other formulations suitable for ocular and aural administration include ointments, biodegradable (i.e., absorbable gel sponges, collagen) and non-biodegradable (i.e., silicone) implants, wafers, lenses and particulate or vesicular systems, such as niosomes or liposomes. A polymer such as crossed linked polyacrylic acid, polyvinyl alcohol, hyaluronic acid, a cellulosic polymer, for example, hydroxypropylmethylcellulose, hydroxyethylcellulose, or methylcellulose, or a heteropolysaccharide polymer, for example, gelan gum, may be incorporated together with a preservative, such as benzalkonium chloride. Such formulations may also be delivered by iontophoresis.

For intranasal administration, the compounds of the invention are conveniently delivered in the form of a solution or suspension from a pump spray container that is squeezed or pumped by the patient or as an aerosol spray presentation from a pressurized container or a nebulizer, with the use of a suitable propellant. Formulations suitable for intranasal administration are typically administered in the form of a dry powder (either alone, as a mixture, for example, in a dry blend with lactose, or as a mixed component particle, for example, mixed with phospholipids, such as phosphatidylcholine) from a dry powder inhaler or as an aerosol spray from a pressurized container, pump, spray, atomizer (preferably an atomizer using electrohydrodynamics to produce a fine mist), or nebulizer, with or without the use of a suitable propellant, such as 1,1,1,2-tetrafluoroethane or 1,1,1,2,3,3,3-heptafluoropropane. For intranasal use, the powder may comprise a bioadhesive agent, for example, chitosan or cyclodextrin.

In another embodiment, the invention comprises a rectal dosage form. Such rectal dosage form may be in the form of, for example, a suppository. Cocoa butter is a traditional suppository base, but various alternatives may be used as appropriate.

Other excipients and modes of administration known in the pharmaceutical art may also be used. Pharmaceutical compositions of the invention may be prepared by any of the well-known techniques of pharmacy, such as effective formulation and administration procedures. The above considerations in regard to effective formulations and administration procedures are well known in the art and are described in standard textbooks. Formulation of drugs is discussed in, for example, Ansel, Howard C., et al., Ansel's Pharmaceutical Dosage Forms and Drug Delivery Systems. Philadelphia: Lippincott, Williams & Wilkins, 2004; Gennaro, Alfonso R., et al. Remington: The Science and Practice of Pharmacy. Philadelphia: Lippincott, Williams & Wilkins, 2000; Rowe, Raymond C. Handbook of Pharmaceutical Excipients. Chicago, Pharmaceutical Press, 2005; Stahl, P. Heinrich and Camilli G. Wermuth, Eds. Handbook of Pharmaceutical Salts: Properties, Selection, and Use. New York: Wiley-VCH, 2011; and Brittain, Harry G., Ed. Polymorphism in Pharmaceutical Solids. New York: Informa Healthcare USA, Inc., 2016.

Acceptable excipients are nontoxic to subjects at the dosages and concentrations employed, and may comprise one or more of the following: 1) buffers such as phosphate, citrate, or other organic acids; 2) salts such as sodium chloride; 3) antioxidants such as ascorbic acid or methionine; 4) preservatives such as octadecyldimethylbenzyl ammonium chloride, hexamethonium chloride, benzalkonium chloride, benzethonium chloride, phenol, butyl or benzyl alcohol; 5) alkyl parabens such as methyl or propyl paraben, catechol, resorcinol, cyclohexanol, 3-pentanol, or m-cresol; 6) low molecular weight (less than about 10 residues) polypeptides; 7) proteins such as serum albumin, gelatin, or immunoglobulins; 8) hydrophilic polymers such as polyvinylpyrrolidone; 9) amino acids such as glycine, glutamine, asparagine, histidine, arginine, or lysine; 10) monosaccharides, disaccharides, or other carbohydrates including glucose, mannose, or dextrins; 11) chelating agents such as EDTA; 12) sugars such as sucrose, mannitol, trehalose or sorbitol; 13) salt-forming counter-ions such as sodium, metal complexes (e.g., Zn-protein complexes), or 14) non-ionic surfactants such as polysorbates (e.g., polysorbate 20 or polysorbate 80), poloxamers or polyethylene glycol (PEG).

Liposome containing compounds of the invention may be prepared by methods known in the art (See, for example, Chang, H. I.; Yeh, M. K.; Clinical development of liposome-based drugs: formulation, characterization, and therapeutic efficacy; Int J Nanomedicine 2012; 7; 49-60). Particularly useful liposomes may be generated by the reverse phase evaporation method with a lipid composition comprising phosphatidylcholine, cholesterol and PEG-derivatized phosphatidylethanolamine (PEG-PE). Liposomes are extruded through filters of defined pore size to yield liposomes with the desired diameter.

Compounds of the invention may also be entrapped in microcapsules prepared, for example, by coacervation techniques or by interfacial polymerization, for example, hydroxymethylcellulose or gelatin-microcapsules and poly-(methylmethacrylate) microcapsules, respectively, in colloidal drug delivery systems (for example, liposomes, albumin microspheres, microemulsions, nano-particles and nanocapsules) or in macroemulsions. Such techniques are disclosed in Remington, The Science and Practice of Pharmacy, 20^{th} Ed., Mack Publishing (2000).

Sustained-release preparations may be used. Suitable examples of sustained-release preparations include semi-permeable matrices of solid hydrophobic polymers containing a compound of the invention, which matrices are in the form of shaped articles, e.g., films, or microcapsules. Examples of sustained-release matrices include polyesters, hydrogels (for example, poly(2-hydroxyethyl-methacrylate), or ‘poly(vinylalcohol)), polylactides, copolymers of L-glutamic acid and 7 ethyl-L-glutamate, non-degradable ethylene-vinyl acetate, degradable lactic acid-glycolic acid copolymers such as those used in leuprolide acetate for depot suspension (injectable microspheres composed of lactic acid-glycolic acid copolymer and leuprolide acetate), sucrose acetate isobutyrate, and poly-D-(−)-3-hydroxybutyric acid.

The formulations to be used for intravenous administration must be sterile. This is readily accomplished by, for example, filtration through sterile filtration membranes. Compounds of the invention are generally placed into a container having a sterile access port, for example, an intravenous solution bag or vial having a stopper pierceable by a hypodermic injection needle.

Suitable emulsions may be prepared using commercially available fat emulsions, such as a lipid emulsions comprising soybean oil, a fat emulsion for intravenous administration (e.g., comprising safflower oil, soybean oil, egg phosphatides and glycerin in water), emulsions containing soya bean oil and medium-chain triglycerides, and lipid emulsions of cottonseed oil. The active ingredient may be either dissolved in a pre-mixed emulsion composition or alternatively it may be dissolved in an oil (e.g., soybean oil, safflower oil, cottonseed oil, sesame oil, corn oil or almond oil) and an emulsion formed upon mixing with a phospholipid (e.g., egg phospholipids, soybean phospholipids or soybean lecithin) and water. It will be appreciated that other ingredients may be added, for example glycerol or glucose, to adjust the tonicity of the emulsion. Suitable emulsions will typically contain up to 20% oil, for example, between 5 and 20%. The fat emulsion may comprise fat droplets between 0.1 and 1.0 μm, particularly 0.1 and 0.5 μm, and have a Ph in the range of 5.5 to 8.0.

For example, the emulsion compositions may be those prepared by mixing a compound of the invention with a lipid emulsions comprising soybean oil or the components thereof (soybean oil, egg phospholipids, glycerol and water).

Compositions for inhalation or insufflation include solutions and suspensions in pharmaceutically acceptable aqueous or organic solvents, or mixtures thereof, and powders. The liquid or solid compositions may contain suitable pharmaceutically acceptable excipients as set out above. In some embodiments, the compositions are administered by the oral or nasal respiratory route for local or systemic effect. Compositions in preferably sterile pharmaceutically acceptable solvents may be nebulized by use of gases. Nebulized solutions may be breathed directly from the nebulizing device or the nebulizing device may be attached to a face mask, tent or intermittent positive pressure breathing machine. Solution, suspension or powder compositions may be administered, preferably orally or nasally, from devices which deliver the formulation in an appropriate manner.

A drug product intermediate (DPI) is a partly processed material that must undergo further processing steps before it becomes bulk drug product. Compounds of the invention may be formulated into drug product intermediate DPI containing the active ingredient in a higher free energy form than the crystalline form. One reason to use a DPI is to improve oral absorption characteristics due to low solubility, slow dissolution, improved mass transport through the mucus layer adjacent to the epithelial cells, and in some cases, limitations due to biological barriers such as metabolism and transporters. Other reasons may include improved solid state stability and downstream manufacturability. In one embodiment, the drug product intermediate contains a compound of the invention isolated and stabilized in the amorphous state (for example, amorphous solid dispersions (ASDs)). There are many techniques known in the art to manufacture ASD's that produce material suitable for integration into a bulk drug product, for example, spray dried dispersions (SDD's), melt extrudates (often referred to as HME's), co-precipitates, amorphous drug nanoparticles, and nano-adsorbates. In one embodiment amorphous solid dispersions comprise a compound of the invention and a polymer excipient. Other excipients as well as concentrations of said excipients and the compound of the disclosure are well known in the art and are described in standard textbooks. See, for example, “Amorphous Solid Dispersions Theory and Practice” by Navnit Shah et al.

### Administration and Dosing

A compound of the invention is administered in an amount effective to treat a condition as described herein. The compounds of the invention may be administered as compound per se, or alternatively, as a pharmaceutically acceptable salt, tautomer, or pharmaceutically acceptable salt of the tautomer. For administration and dosing purposes, the compound per se, pharmaceutically acceptable salt, tautomer, or pharmaceutically acceptable salt of the tautomer thereof will simply be referred to as the compounds of the invention.

The compounds of the invention are administered by any suitable route in the form of a pharmaceutical composition adapted to such a route, and in a dose effective for the treatment intended. The compounds of the invention may be administered orally, rectally, vaginally, parenterally, topically, intranasally, or by inhalation.

In a preferred embodiment, the compounds of the invention may be administered orally.

Oral administration may involve swallowing, so that the compound enters the gastrointestinal tract, or buccal or sublingual administration may be employed by which the compound enters the bloodstream directly from the mouth.

In another embodiment, the compounds of the invention may also be administered parenterally, for example directly into the bloodstream, into muscle, or into an internal organ. Suitable means for parenteral administration include intravenous, intraarterial, intraperitoneal, intrathecal, intraventricular, intraurethral, intrasternal, intracranial, intramuscular and subcutaneous. Suitable devices for parenteral administration include needle (including microneedle) injectors, needle-free injectors, and infusion techniques.

In another embodiment, the compounds of the invention may also be administered topically to the skin or mucosa, that is, dermally or transdermally. In another embodiment, the compounds of the invention may also be administered intranasally or by inhalation. In another embodiment, the compounds of the invention may be administered rectally or vaginally. In another embodiment, the compounds of the invention may also be administered directly to the eye or ear.

The dosage regimen for the compounds of the invention or compositions containing said compounds is based on a variety of factors, including the type, age, weight, sex and medical condition of the patient; the severity of the condition; the route of administration; and the activity of the particular compound employed. Thus, the dosage regimen may vary widely. In one embodiment, the total daily dose of a compound of the invention is typically from about 0.01 to about 100 mg/kg (i.e., mg compound of the invention per kg body weight) for the treatment of the indicated conditions discussed herein. In another embodiment, total daily dose of the compound of the invention is from about 0.1 to about 50 mg/kg, and in another embodiment, from about 0.5 to about 30 mg/kg. It is not uncommon that the administration of the compounds of the invention will be repeated a plurality of times in a day (typically no greater than 4 times). Multiple doses per day typically may be used to increase the total daily dose, if desired. In some embodiments, the compound, pharmaceutically acceptable salt, tautomer, or pharmaceutically acceptable salt of the tautomer thereof is administered once a day, twice a day, or three times a day. In a preferred embodiment, the compound, pharmaceutically acceptable salt, tautomer, or pharmaceutically acceptable salt of the tautomer thereof is administered once a day. In a preferred embodiment, the compound, pharmaceutically acceptable salt, tautomer, or pharmaceutically acceptable salt of the tautomer thereof is administered twice a day. In a preferred embodiment, the compound, pharmaceutically acceptable salt, tautomer, or pharmaceutically acceptable salt of the tautomer thereof is administered three times a day.

In one embodiment, a compound of the disclosure, a pharmaceutically acceptable salt thereof, a tautomer thereof, or a pharmaceutically acceptable salt of the tautomer thereof, or a pharmaceutical composition comprising the compound or a pharmaceutically acceptable salt thereof, a tautomer thereof, or a pharmaceutically acceptable salt of the tautomer thereof, may be administered orally in the form of a tablet or a capsule. The dosage of the compound, pharmaceutically acceptable salt, tautomer, or pharmaceutically acceptable salt of the tautomer thereof may be adjusted based on the patient's response and symptoms. In some embodiments, the compound or pharmaceutical composition may provide the compound in an amount of from about 0.01 mg to about 150 mg, from about 150 mg to about 250 mg, from about 250 mg to about 500 mg, from about 500 mg to about 750 mg, from about 750 mg to about 1000 mg, from about 1250 mg to about 1500 mg, from about 1500 mg to about 1750 mg, from about 1750 mg to about 2000 mg, from about 2000 mg to about 2250 mg, from about 2250 mg to about 2500 mg, from about 2500 mg to about 2750 mg, from about 2750 mg to about 3000 mg, from about 3000 mg to about 3250 mg, from about 3250 mg to about 3500 mg, from about 3500 mg to about 3750 mg, from about 3750 mg to about 4000 mg, from about 4000 mg to about 4250 mg, from about 4250 mg to about 4500 mg, from about 4500 mg to about 4750 mg, or from about 4750 mg to about 5000 mg. In a preferred embodiment, the compound, pharmaceutically acceptable salt, tautomer, or pharmaceutically acceptable salt of the tautomer thereof may be provided in an amount of from about 1 mg to about 2500 mg. In a preferred embodiment, the compound, pharmaceutically acceptable salt, tautomer, or pharmaceutically acceptable salt of the tautomer thereof may be provided in an amount of from about 1 mg to about 100 mg. In a preferred embodiment, the compound, pharmaceutically acceptable salt, tautomer, or pharmaceutically acceptable salt of the tautomer thereof may be provided in an amount of from about 1 mg to about 50 mg. In a preferred embodiment, the compound, pharmaceutically acceptable salt, tautomer, or pharmaceutically acceptable salt of the tautomer thereof may be provided in an amount of from about 1 mg to about 25 mg. In some embodiments, the compound, pharmaceutically acceptable salt, tautomer, or pharmaceutically acceptable salt of the tautomer thereof may be provided in an amount of from about 150 mg to about 2500 mg. In some embodiments, the compound, pharmaceutically acceptable salt, tautomer, or pharmaceutically acceptable salt of the tautomer thereof may be provided in an amount of from about 150 mg to about 500 mg. In a preferred embodiment, the compound, pharmaceutically acceptable salt, tautomer, or pharmaceutically acceptable salt of the tautomer thereof may be provided in an amount of from about 100 mg to about 1000 mg. In some embodiments, the compound, pharmaceutically acceptable salt, tautomer, or pharmaceutically acceptable salt of the tautomer thereof may be provided in an amount of from about 500 mg to about 1500 mg. In some embodiments, the compound, pharmaceutically acceptable salt, tautomer, or pharmaceutically acceptable salt of the tautomer thereof may be provided in an amount of from about 1500 mg to about 2500 mg. In some embodiments, the compound, pharmaceutically acceptable salt, tautomer, or pharmaceutically acceptable salt of the tautomer thereof may be provided in an amount of from about 2500 mg to about 5000 mg.

A compound, pharmaceutically acceptable salt, tautomer, or pharmaceutically acceptable salt of the tautomer thereof disclosed herein may be provided in an amount of about 0.01 mg, about 1 mg, about 2 mg, about 3 mg, about 4 mg, about 5 mg, about 6 mg, about 7 mg, about 8 mg, about 9 mg, about 10 mg, about 11 mg, about 12 mg, about 13 mg, about 14 mg, about 15 mg, about 16 mg, about 17 mg, about 18 mg, about 19 mg, about 20 mg, about 50 mg, about 100 mg, about 150 mg, about 200 mg, about 250 mg, about 250 mg, about 300 mg, about 350 mg, about 400 mg, about 450 mg, about 500 mg, about 550 mg, about 600 mg, about 650 mg, about 700 mg, about 750 mg, about 800 mg, about 850 mg, about 900 mg, about 950 mg, about 1000 mg, about 1100 mg, about 1200 mg, about 1300 mg, about 1400 mg, about 1500 mg, about 1600 mg, about 1700 mg, about 1800 mg, about 1900 mg, about 2000 mg, about 2100 mg, about 2200 mg, about 2300 mg, about 2400 mg, about 2500 mg, about 2600 mg, about 2700 mg, about 2800 mg, about 2900 mg, about 3000 mg, about 3200 mg, about 3400 mg, about 3600 mg, about 3800 mg, about 4000 mg, about 4200 mg, about 4400 mg, about 4600 mg, about 4800 mg, or about 5000 mg. In a preferred embodiment, the compound, pharmaceutically acceptable salt, tautomer, or pharmaceutically acceptable salt of the tautomer thereof disclosed herein may be provided in an amount of about 5 mg. In a preferred embodiment, the compound, pharmaceutically acceptable salt, tautomer, or pharmaceutically acceptable salt of the tautomer thereof disclosed herein may be provided in an amount of about 10 mg. In a preferred embodiment, the compound, pharmaceutically acceptable salt, tautomer, or pharmaceutically acceptable salt of the tautomer thereof disclosed herein may be provided in an amount of about 15 mg. In a preferred embodiment, the compound, pharmaceutically acceptable salt, tautomer, or pharmaceutically acceptable salt of the tautomer thereof disclosed herein may be provided in an amount of about 25 mg. In a preferred embodiment, the compound, pharmaceutically acceptable salt, tautomer, or pharmaceutically acceptable salt of the tautomer thereof disclosed herein may be provided in an amount of about 50 mg. In a preferred embodiment, the compound, pharmaceutically acceptable salt, tautomer, or pharmaceutically acceptable salt of the tautomer thereof disclosed herein may be provided in an amount of about 75 mg. In a preferred embodiment, the compound, pharmaceutically acceptable salt, tautomer, or pharmaceutically acceptable salt of the tautomer thereof disclosed herein may be provided in an amount of about 100 mg. In a preferred embodiment, the compound, pharmaceutically acceptable salt, tautomer, or pharmaceutically acceptable salt of the tautomer thereof disclosed herein may be provided in an amount of about 250 mg. In a preferred embodiment, the compound, pharmaceutically acceptable salt, tautomer, or pharmaceutically acceptable salt of the tautomer thereof disclosed herein may be provided in an amount of about 500 mg. In a preferred embodiment, the compound, pharmaceutically acceptable salt, tautomer, or pharmaceutically acceptable salt of the tautomer thereof disclosed herein may be provided in an amount of about 1000 mg.

### Therapeutic Methods and Uses

The compounds of the disclosure may activate AMPK and may be useful in the treatment of a condition associated with AMPK. In a preferred embodiment, the compounds of the disclosure may be a pan-AMPK activator and may be useful in the treatment of a condition associated with AMPK. In some embodiments, the condition or disorder is a metabolic disorder, an inflammatory disorder, an autoimmune disorder, a disorder of gastrointestinal barrier dysfunction, a functional gastrointestinal disorder, an eating disorder, a nutritional disorder, an allergy, or a central nervous system (CNS) disorder. In a preferred embodiment, the AMPK-activating compounds of the disclosure may be administered to a subject in need thereof to treat a metabolic disorder. In a preferred embodiments, the AMPK-activating compounds of the disclosure may be administered to a subject in need thereof to treat an inflammatory or autoimmune disorder. In a preferred embodiments, the AMPK-activating compounds of the disclosure may be administered to a subject in need thereof to treat a disorder of gastrointestinal barrier dysfunction or a functional gastrointestinal disorder.

In some embodiments, the AMPK-activating compounds disclosed herein may treat a metabolic disorder or a complication resulting from a metabolic condition selected from the group consisting of type 2 diabetes, gestational diabetes, insulin resistance, hyperglycemia, hypercholesterolemia, hypertriglyceridemia (elevated levels of triglyceride-rich-lipoproteins), obesity, abdominal obesity, vascular restenosis, hyperinsulinemia, glucose intolerance, atherosclerosis, Metabolic Syndrome, hypertension, high hepatic glucose output, high blood glucose concentrations, nonalcoholic steatohepatitis (NASH), dyslipidemia, mixed dyslipidemia, diabetic dyslipidemia, protection against ischemia and reperfusion damage, a lipid disorder, elevated levels of plasma triglycerides, elevated levels of free fatty acids, elevated levels of cholesterol, high levels of low density lipoprotein (LDL), low levels of high density lipoprotein (HDL), chronic kidney disease, diabetic nephropathy, diabetic retinopathy, diabetic neuropathy, cardiovascular disease, hypoxia, cancer, non-alcoholic fatty liver disease (NAFLD), glucocorticoid-induced apoptosis, loss of skeletal muscle mass, sarcopenia, high circulating free fatty acids (FFAs), heart attack, cardiomyopathy, heart failure, and atherosclerosis. In a preferred embodiment, the AMPK activating compounds of the disclosure may treat a metabolic disorder selected from the group consisting of type 2 diabetes, gestational diabetes, hyperglycemia, metabolic syndrome, obesity, hypercholesterolemia, or hypertension.

In some embodiments, the AMPK activating compounds disclosed herein may treat an inflammatory disorder or an autoimmune disorder selected from the group consisting of inflammatory bowel disease, ulcerative colitis, Crohn's disease, checkpoint inhibitor-induced colitis, psoriasis, celiac disease, graft-versus-host disease (GVHD), radiation-induced enteritis, chemotherapy-induced enteritis, and necrotizing enterocolitis. In some embodiments, the AMPK-activating compounds disclosed herein may treat gastrointestinal injury resulting from toxic insults such as radiation or chemotherapy. In a preferred embodiment, the AMPK-activating compounds of the disclosure may treat an inflammatory disorder or an autoimmune disorder selected from the group consisting of inflammatory bowel disease, colitis, ulcerative colitis, and Crohn's disease. In a preferred embodiment, the AMPK-activating compounds of the disclosure may treat inflammatory bowel disease. In a preferred embodiment, the AMPK-activating compounds of the disclosure may treat colitis. In a preferred embodiment, the AMPK-activating compounds of the disclosure may treat ulcerative colitis. In a preferred embodiment, the AMPK-activating compounds of the disclosure may treat Crohn's disease.

In some embodiments, the AMPK-activating compounds disclosed herein may treat a disorder of gastrointestinal barrier dysfunction, such as environmental enteric dysfunction or spontaneous bacterial peritonitis. In some embodiments, the AMPK-activating compounds disclosed herein may treat ischemic colitis or sclerosing cholangitis.

In some embodiments, the AMPK-activating compounds disclosed herein may treat a functional gastrointestinal disorder selected from the group consisting of irritable bowel syndrome, functional dyspepsia, functional abdominal bloating, functional abdominal distension, functional diarrhea, functional constipation, gastroparesis, disorders related to microbiome dysbiosis, and opioid-induced constipation. In a preferred embodiment, the AMPK-activating compounds of the disclosure may treat a functional gastrointestinal disorder selected from the group consisting of irritable bowel syndrome, functional diarrhea, celiac disease, and functional constipation.

In some embodiments, the AMPK-activating compounds disclosed herein may treat an eating disorder or a nutritional disorder selected from the group consisting of hyperphagia, cachexia, anorexia nervosa, short bowel syndrome, intestinal failure, and intestinal insufficiency. In some embodiments, the AMPK-activating compounds of the disclosure may treat complications associated with an eating disorder or a nutritional disorder, such as left ventricular hypertrophy.

In some embodiments, the AMPK-activating compounds disclosed herein may treat an allergy, such as food allergies and celiac sprue. In some embodiments, the AMPK-activating compounds disclosed herein may treat nausea and vomiting.

In some embodiments, the AMPK-activating compounds disclosed herein may treat a central nervous system disorder selected from the group consisting of a mood disorder, anxiety, depression, an affective disorder, schizophrenia, malaise, cognition disorder, addiction, autism, epilepsy, a neurodegenerative disorder, Alzheimer's disease, Parkinson's disease, Lewy Body dementia, episodic cluster headaches, migraines, and pain.

In some embodiments, the AMPK-activating compounds of the disclosure can decrease fatty acid synthesis; increase fatty acid oxidation; increase ketogenesis; decrease cholesterol synthesis, lipogenesis, and/or triglyceride synthesis; decrease blood glucose levels and/or concentrations; improve glucose homeostasis; normalize glucose metabolism; decrease blood pressure; increase HDL levels; decrease LDL levels; decrease plasma triglyceride levels; decrease fatty acid levels; decrease hepatic glucose output; improve insulin action; decrease blood pressure; improve insulin sensitivity; suppress hepatic glucose output; inhibit de novo lipogenesis; simulate muscle glucose uptake; modulate insulin secretion by pancreatic p cells; decrease body weight; increase skeletal muscle mass; or prevent the loss of skeletal muscle mass. In a preferred embodiment, the AMPK-activating compounds disclosed herein can treat or reduce systemic infection or systemic inflammation from having a leaky gut barrier. In a preferred embodiment, the AMPK-activating compounds disclosed herein are pan-AMPK activators.

### Co-Administration

The compounds of the invention may be used alone, or in combination with one or more other therapeutic agents. The invention provides any of the uses, methods or compositions as defined herein wherein the compound of the invention, pharmaceutically acceptable salt, tautomer, or pharmaceutically acceptable salt of the tautomer thereof, is used in combination with one or more other therapeutic agent discussed herein.

The administration of two or more compounds “in combination” means that all of the compounds are administered closely enough in time to affect treatment of the subject. The two or more compounds may be administered simultaneously or sequentially, via the same or different routes of administration, on same or different administration schedules and with or without specific time limits depending on the treatment regimen. Additionally, simultaneous administration may be carried out by mixing the compounds prior to administration or by administering the compounds at the same point in time but as separate dosage forms at the same or different site of administration. Examples of “in combination” include, but are not limited to, “concurrent administration,” “co-administration,” “simultaneous administration,” “sequential administration” and “administered simultaneously”.

A compound of the invention and the one or more other therapeutic agents may be administered as a fixed or non-fixed combination of the active ingredients. The term “fixed combination” means a compound of the invention, pharmaceutically acceptable salt, tautomer, or pharmaceutically acceptable salt of the tautomer thereof, and the one or more therapeutic agents, are both administered to a subject simultaneously in a single composition or dosage. The term “non-fixed combination” means that a compound of the invention, pharmaceutically acceptable salt, tautomer, or pharmaceutically acceptable salt of the tautomer thereof, and the one or more therapeutic agents are formulated as separate compositions or dosages such that they may be administered to a subject in need thereof simultaneously or at different times with variable intervening time limits, wherein such administration provides effective levels of the two or more compounds in the body of the subject.

In one embodiment, the compounds of this invention are administered in combination with the specifically named agents including the pharmaceutically acceptable salts of the specifically named agents and the pharmaceutically acceptable solvates of said agents and salts.

The present invention also provides any of the uses, methods or compositions as defined above wherein the compound of Formula I, Ia, II, III, IVa-c, and V, pharmaceutically acceptable salt, tautomer, or pharmaceutically acceptable salt of the tautomer thereof, is used in combination with another pharmacologically active compound, particularly one of the functionally-defined classes or specific compounds listed below. These agents may be administered as part of the same or separate dosage forms, via the same or different routes of administration, and on the same or different administration schedules according to standard pharmaceutical practice known to one skilled in the art.

Suitable agents for use in combination therapy with a compound of Formula I, Ia, II, III, IVa-c, and V, pharmaceutically acceptable salt, tautomer, or pharmaceutically acceptable salt of the tautomer thereof, include: sulfasalazine, mesalazine, prednisone, azathioprine, infliximab, adalimumab, belimumab, becertolizumab, natalizumab, vedolizumab, hydrocortisone, budesonide, cyclosporin, tacrolimus, fexofenadine, 6-mercaptopurine, methotrexate, ursodeoxycholic acid, obeticholic acid, anti-histamines, rifampin, prednisone, methotrexate, azathioprine, cyclophosphamide, hydroxychloroquine, mofetil, sodium mycophenolate, tacrolimus, leflunomide, chloroquine and quinacrine, thalidomide, rituxan, NSAIDs, solumedrol, depomedrol, and dexamethasone.

Other suitable agents for use in combination therapy with a compound of Formula I, Ia, II, III, IVa-c, and V, pharmaceutically acceptable salt, tautomer, or pharmaceutically acceptable salt of the tautomer thereof, include: a 5-lipoxygenase activating protein (FLAP) antagonist; a leukotriene antagonist (LTRA) such as an antagonist of LTB₄, LTC₄, LTD₄, LTE₄, CysLT₁ or CysLT₂, e.g., montelukast or zafirlukast; a histamine receptor antagonist, such as a histamine type 1 receptor antagonist or a histamine type 2 receptor antagonist, e.g., loratidine, fexofenadine, desloratidine, levocetirizine, methapyrilene or cetirizine; an α1-adrenoceptor agonist or an α2-adrenoceptor agonist, e.g., phenylephrine, methoxamine, oxymetazoline or methylnorephrine; a muscarinic M3 receptor antagonist, e.g., tiotropium or ipratropium; a dual muscarinic M3 receptor antagononist/β2 agonist; a PDE inhibitor, such as a PDE3 inhibitor, a PDE4 inhibitor or a PDE5 inhibitor, e.g., theophylline, sildenafil, vardenafil, tadalafil, ibudilast, cilomilast or roflumilast; sodium cromoglycate or sodium nedocromil; a cyclooxygenase (COX) inhibitor, such as a non-selective inhibitor (e.g., aspirin or ibuprofen) or a selective inhibitor (e.g., celecoxib or valdecoxib); a glucocorticosteroid, e.g., fluticasone, mometasone, dexamethasone, prednisolone, budesonide, ciclesonide or beclamethasone; an anti-inflammatory monoclonal antibody, e.g., infliximab, adalimumab, tanezumab, ranibizumab, bevacizumab or mepolizumab; a β2 agonist, e.g., salmeterol, albuterol, salbutamol, fenoterol or formoterol, particularly a long-acting β2 agonist; an integrin antagonist, e.g., natalizumab; an adhesion molecule inhibitor, such as a VLA-4 antagonist; a kinin B₁ or B₂ receptor antagonist; an immunosuppressive agent, such as an inhibitor of the IgE pathway (e.g., omalizumab) or cyclosporine; a matrix metalloprotease (MMP) inhibitor, such as an inhibitor of MMP-9 or MMP-12; a tachykinin NK₁, NK₂ or NK₃ receptor antagonist; a protease inhibitor, such as an inhibitor of elastase, chymase or catheopsin G; an adenosine A₂ₐ receptor agonist; an adenosine A_{2b} receptor antagonist; a urokinase inhibitor; a dopamine receptor agonist (e.g., ropinirole), particularly a dopamine D2 receptor agonist (e.g., bromocriptine); a modulator of the NFκB pathway, such as an IKK inhibitor; a further modulator of a cytokine signaling pathway such as an inhibitor of syk kinase, p38 kinase, SPHK-1 kinase, Rho kinase, EGF-R or MK-2; a mucolytic, mucokinetic or anti-tussive agent; an antibiotic; an antiviral agent; a vaccine; a chemokine; an epithelial sodium channel (ENaC) blocker or Epithelial sodium channel (EnaC) inhibitor; a nucleotide receptor agonist, such as a P2Y2 agonist; a thromboxane inhibitor; niacin; a 5-lipoxygenase (5-LO) inhibitor, e.g., Zileuton; an adhesion factor, such as VLAM, ICAM or ELAM; a CRTH2 receptor (DP₂) antagonist; a prostaglandin D₂ receptor (DP₁) antagonist; a hematopoietic prostaglandin D2 synthase (HPGDS) inhibitor; interferon-β; a soluble human TNF receptor, e.g., Etanercept; a HDAC inhibitor; a phosphoinositide 3-kinase gamma (PI3Kγ) inhibitor; a phosphoinositide 3-kinase delta (PI3Kδ) inhibitor; a CXCR-1 or a CXCR-2 receptor antagonist; an IRAK-4 inhibitor; and, a TLR-4 or TLR-9 inhibitor, including the pharmaceutically acceptable salts of the specifically named compounds. The agents may be administered with another active agent, wherein the second active agent may be administered either orally or topically.

These agents and compounds of the invention may be combined with pharmaceutically acceptable vehicles such as saline, Ringer's solution, dextrose solution, and the like. The particular dosage regimen, i.e., dose, timing and repetition, will depend on the particular individual and that individual's medical history.

### Kits

Another aspect of the invention provides kits comprising the compound of the invention or pharmaceutical compositions comprising the compound of the invention. A kit may include, in addition to the compound of the invention or pharmaceutical composition thereof, diagnostic or therapeutic agents. A kit may also include instructions for use in a diagnostic or therapeutic method. In some embodiments, the kit includes the compound or a pharmaceutical composition thereof and a diagnostic agent. In other embodiments, the kit includes the compound or a pharmaceutical composition thereof and one or more therapeutic agents, such as the therapeutic agents for co-administration described herein.

In yet another embodiment, the invention comprises kits that are suitable for use in performing the methods of treatment described herein. In one embodiment, the kit contains a first dosage form comprising one or more of the compounds of the invention in quantities sufficient to carry out the methods of the invention. In another embodiment, the kit comprises one or more compounds of the invention in quantities sufficient to carry out the methods of the invention and a container for the dosage and a container for the dosage.

### Synthetic Methods

Compounds of the present invention may be synthesized by synthetic routes that include processes analogous to those well-known in the chemical arts, particularly in light of the description contained herein. The starting materials are generally available from commercial sources or may be prepared using methods well known to those skilled in the art. Many of the compounds used herein, are related to, or may be derived from compounds in which one or more of the scientific interest or commercial need has occurred. Accordingly, such compounds may be one or more of 1) commercially available; 2) reported in the literature or 3) prepared from other commonly available substances by one skilled in the art using materials which have been reported in the literature.

Detailed descriptions of individual reaction steps are detailed in the EXAMPLES section below. Those skilled in the art will appreciate that other synthetic routes may be used to synthesize the inventive compounds. Although specific starting materials and reagents are discussed below, other starting materials and reagents may be substituted to provide one or more of a variety of derivatives or reaction conditions. In addition, many of the compounds prepared by the methods described below may be further modified in light of this disclosure using conventional chemistry well known to those skilled in the art.

The skilled person will appreciate that the experimental conditions set forth in the Preparations and Examples that follow are illustrative of suitable conditions for effecting the transformations shown, and that it may be necessary or desirable to vary the precise conditions employed for the preparation of compounds of the invention. It will be further appreciated that it may be necessary or desirable to carry out the transformations in a different order from that described in the Preparations and Examples, or to modify one or more of the transformations, to provide the desired compound of the invention.

In the preparation of compounds of the invention it is noted that some of the preparation methods useful for the preparation of the compounds described herein may require protection of remote functionality (e.g., a primary amine, secondary amine, carboxyl, etc. in a precursor of a compound of the invention). The need for such protection will vary depending on the nature of the remote functionality and the conditions of the preparation methods. The need for such protection is readily determined by one skilled in the art. The use of such protection/deprotection methods is also within the skill in the art. For a general description of protecting groups and their use, see March's Advanced Organic Chemistry: Reactions, Mechanisms, and Structure 8^{th} Edition.

For example, if a compound contains a amine or carboxylic acid functionality, such functionality may interfere with reactions at other sites of the molecule if left unprotected. Accordingly, such functionalities may be protected by an appropriate protecting group (PG) which may be removed in a subsequent step. Suitable protecting groups for amine and carboxylic acid protection include those protecting groups commonly used in peptide synthesis (such as N-t-butoxycarbonyl (Boc), benzyloxycarbonyl (Cbz), and 9-fluorenylmethylenoxycarbonyl (Fmoc) for amines and lower alkyl or benzyl esters for carboxylic acids) which are generally not chemically reactive under the reaction conditions described and may typically be removed without chemically altering other functionality in a compound of the invention.

### General Experimental Details

In the non-limiting Examples and Preparations that illustrate the invention and that are set out in the description and the following schemes, the following abbreviations, definitions and analytical procedures may be referred to. Other abbreviations common in the art may also be used. Compounds of the present invention were named using ChemDraw Professional™ version 20 (Perkin Elmer) or were given names which are consistent with IUPAC nomenclature.

¹H Nuclear magnetic resonance (NMR) spectra were in all cases consistent with the proposed structures. Characteristic chemical shifts (b) are given in parts-per-million downfield from tetramethylsilane using conventional abbreviations for designation of major peaks: e.g., s, singlet; d, doublet; t, triplet; q, quartet; quin, quintet; m, multiplet; br, broad. The following abbreviations have been used for common NMR solvents: CD₃CN, deuteroacetonitrile; CDCl₃, deuterochloroform; DMSO-d₆, deuterodimethylsulfoxide; and MeOD, deuteromethanol. Where appropriate, tautomers may be recorded within the NMR data; and some exchangeable protons may not be visible. Some resonances in the NMR spectrum appear as complex multiplets because the isolate is a mixture of two conformers.

Mass spectra were recorded using electron impact ionization (EI), electrospray ionization (ESI) or atmospheric pressure chemical ionization (APCI). The observed ions are reported as MS m/z and may be positive ions of the compound [M]⁺, compound plus a proton [M+H]⁺, or compound plus a sodium ion [M+Na]⁺. In some cases the only observed ions may be fragment ions reported as [M+H-(fragment lost)]+. Where relevant, the reported ions are assigned for isotopes of chlorine (³⁵C₁ and/or ³⁷Cl), bromine (⁷⁹Br and/or ⁸¹Br) and tin (¹²⁰Sn).

Wherein TLC, chromatography or HPLC has been used to purify compounds, one skilled in the art may choose any appropriate solvent or combination of solvents to purify the desired compound. Chromatographic separations (excluding HPLC) were carried out using silica gel adsorbent unless otherwise noted.

All reactions were carried out using continuous stirring under an atmosphere of nitrogen or argon gas unless otherwise noted. In some cases, reactions were purged with nitrogen or argon gas prior to the start of the reaction. In these cases, the nitrogen or argon gas was bubbled through the liquid phase of the mixture for the approximate specified time. Solvents used were commercial anhydrous grades. All starting materials were commercially available products. In some cases, starting materials were prepared according to reported literature procedures. It will be apparent to one skilled in the art that the word “concentrated” as used herein generally refers to the practice of evaporation of solvent under reduced pressure, typically accomplished using a rotary evaporator.

### ABBREVIATIONS

Ac₂O is acetic anhydride;
B₂(Pin)₂ is bis(pinacolato)diboron;
br is broad;
tBu is tert-butyl;
° C. is degrees Celcius;
CDCl₃ is deuterochloroform;
δ is chemical shift;
d is doublet;
dd is doublet of doublets;
ddd is doublet of doublet of doublets;
dt is doublet of triplets;
DCM is dichloromethane; methylene chloride;
DMF is N,N-dimethylformamide;
DMSO-d₆ is deuterodimethylsulfoxide;
EtOAc is ethyl acetate;
EtOH is ethanol;
Et₃N is triethylamine;
g is gram;
HPLC is high pressure liquid chromatography;
h is hour(s);
KOAc is potassium acetate;
L is liter;
LC is liquid chromatography;
m is multiplet;
M is molar;
(M−H)⁻ is negative ion of compound minus proton;
MeOD is deuterated methanol;
MeOH is methanol;
mg is milligram;
MHz is mega Hertz;
min(s) is minute(s);
mL is milliliter;
mm is millimeters;
mM is millimolar;
mmol is millimole;
MS is mass spectrometry;
(M+H)⁺ is positive ion of compound plus proton;
NMR is nuclear magnetic resonance;
Pd₂(dba)₃ is tris(dibenzylideneacetone)dipalladium(0);
Pd(dppf)Cl₂ is [1,1′-bis(diphenylphophino)ferrocene]dichloropalladium(II);
Pd(dppf)Cl₂—CH₂Cl₂ is [1,1′-bis(diphenylphosphino)ferrocene]dichloropalladium(II) dichloromethane complex;
Pd(OAc)₂ is palladium(II) acetate;
Pd(PPh₃)₄ is tetrakis(triphenylphosphine)palladium(0);
PPh₃ is triphenylphosphine;
ppm is parts per million;
psi is pounds per square inch;
q is quartet;
r.t. is retention time;
s is singlet;
SFC is supercritical fluid chromatography;
t is triplet;
TFA is trifluoroacetic acid;
THF is tetrahydrofuran;
TLC is thin layer chromatography;
μ is micron;
μL is microliters;
UPLC is ultra-performance liquid chromatography; and
Xantphos is 4,5-bis(diphenylphosphino)-9,9-dimethylxanthene.

The Schemes described below are intended to provide a general description of the methodology employed in the preparation of the compounds of the present invention. In the following Schemes, the general methods for the preparation of the compounds are shown either in racemic or enantioenriched form. It will be apparent to one skilled in the art that all of the synthetic transformations may be conducted in a precisely similar manner whether the materials are enantioenriched or racemic. Moreover, the resolution to the desired optically active material may take place at any desired point in the sequence using well known methods such as described herein and in the chemistry literature.

Reaction Scheme IA outlines general procedures for the synthesis of compounds of Formula (I). In Reaction Scheme IA, the chemical groups are as defined in the Specification; additionally in Reaction Scheme IA: X=a halide suitable for transition metal catalyzed cross-coupling reactions, preferably iodo, bromo, or chloro; R=a carbon-linked (hetero)alkyl or (hetero)aryl group where —C(O)OR is an ester that is chemically inert to the described reaction cross-coupling reactions but where —C(O)OR may be readily converted to —C(O)OR¹ to provide compounds of Formula (I), preferably R=methyl, ethyl, benzyl, or tert-butyl; —BY₂=a boronic acid or boronate ester suitable for transition metal catalyzed cross-coupling reactions, preferably boronic acid or pinacolboronate.

Heteroaryl halide Intermediates (1a) can be prepared based on methods known in the literature (Tetrahedron Letters 2007, 48, 2457; WO2011008572; Bioorganic & Medicinal Chemistry 2014, 22, 1156; Journal of Organic Chemistry 2023, 88, 13049) in combination with standard functional group interconversions that are described in Reaction Scheme II, Reaction Scheme III, and Reaction Scheme IV. Boronate Intermediates (1b) can be prepared as described in WO2011061168 or WO2014140078. The conversion of heteroaryl halide Intermediates (1a) to boronate Intermediates (1d) can be readily achieved when A¹=CR^{a} by methods known in the literature (WO2018229543; WO2022221526; WO2012119046). Halide Intermediates (1e) can be prepared by methods widely reported in the literature. Intermediates (1c) can be prepared by transition metal catalyzed cross-coupling of halide and boronate Intermediates (1a) and (1b), or (1e) and (1d), using procedures analogous to ACS Medicinal Chemistry Letters 2020, 11, 825; Journal of Medicinal Chemistry 2021, 64, 4498; WO2019201297; WO2016164285; Journal of Medicinal Chemistry 2014, 57, 5129; WO2019213570. Compounds of Formula (I) can be prepared by transesterification or hydrolysis of Intermediates (1c) based upon procedures well known in the literature (WO2012137089; Tetrahedron Letters 2018, 59, 2917; WO2022150574; WO2022229341).

The borylation of Intermediate (1a) to afford Intermediate (1d) may be effected under standard palladium-catalyzed reaction conditions, using a catalyst such as [1,1′-bis(diphenylphosphino)ferrocene]dichloropalladium(II), a base such as potassium acetate, and a borylating reagent such as bis(pinacolato)diboron, in a suitable solvent such as dioxane, at a temperature preferably between 80-110° C. The cross-coupling between halide Intermediate (1a) and boronate Intermediate (1b), or between halide Intermediate (1e) and boronate Intermediate (1d), to afford Intermediate (1c) may be effected using a catalyst such as bis(triphenylphosphine)palladium(II) dichloride or [1,1′-bis(diphenylphosphino)ferrocene]dichloro-palladium(II) and a base such as potassium fluoride, potassium carbonate, or sodium bicarbonate, in a suitable solvent such as aqueous dioxane or aqueous 1,2-dimethoxyethane, at an appropriate temperature, preferably between 80-110° C. The conversion of ester Intermediate (1c) to a compound of Formula (I) may be effected under standard conditions for cleavage of the —C(O)OR ester. For R=small alkyl, such as methyl or ethyl, hydrolysis to afford carboxylic acid of Formula (I) may be effected with a suitable metal hydroxide, such as sodium hydroxide, potassium hydroxide, or lithium hydroxide, in a solvent such as water, or a mixed aqueous-organic solvent such as aqueous methanol, ethanol, and/or tetrahydrofuran at an appropriate temperature, preferably between 0-110° C. For R=tert-butyl, acid-promoted cleavage of the tert-butyl ester to afford carboxylic acid of Formula (I) may be effected with a suitable acid and solvent combination, such as trifluoroacetic acid in dichloromethane, or hydrogen chloride in dioxane, at an appropriate temperature, preferably between 0-30° C.

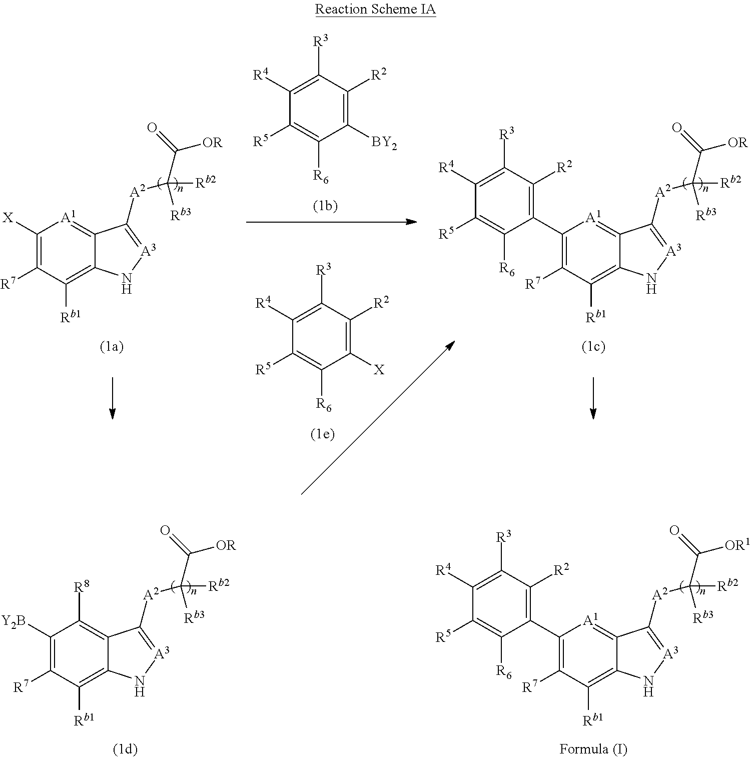

In Reaction Scheme IB, the chemical groups are as defined in the Specification; additionally in Reaction Scheme IB: X=a halide suitable for transition metal catalyzed cross-coupling reactions, preferably iodo, bromo, or chloro; R=a carbon-linked (hetero)alkyl or (hetero)aryl group where —C(O)OR is an ester that is chemically inert to the described reaction cross-coupling reactions but where —C(O)OR may be readily converted to —C(O)OR¹ to provide compounds of Formula (I), preferably R=methyl, ethyl, benzyl, or tert-butyl; —BY₂=a boronic acid or boronate ester suitable for transition metal catalyzed cross-coupling reactions, preferably boronic acid or pinacolboronate.

Reaction Scheme IB depicts an alternate route to Intermediates (1c) via introduction of an alternative R⁴ group. Although Reaction Scheme IB depicts variants of the R⁴ group, one skilled in the art will recognize that analogous procedures may be applied to prepare analogous derivatives at any of the R²-R⁶ substituents. Bis(boronate) Intermediates (1f) can be prepared according to literature references (Chemistry—An Asian Journal 2013, 8, 1368; Organometallics 2002, 21, 4886; Organometallics 2014, 33, 1291). The mono-boronate Intermediates (1g) can be prepared by a selective mono-cross-coupling of bis(boronate) Intermediates (1f) with heteroaryl halide Intermediates (1a) (ACS Macro Letters 2012, 1, 392; WO2016115360; US20160072072; ACS Catalysis 2021, 11, 5968). The Intermediates (1c) can be prepared by transition metal catalyzed cross-coupling of boronate Intermediates (1g) with appropriate (heteroaryl)halides Intermediates (1h) using procedures analogous to ACS Medicinal Chemistry Letters 2020, 11, 825; Journal of Medicinal Chemistry 2021, 64, 4498; WO2019201297; WO2016164285; Journal of Medicinal Chemistry 2014, 57, 5129; WO2019213570.

The cross-coupling between halide Intermediate (1a) and (bis)boronate Intermediate (1f) may be effected using a catalyst such as tetrakis(triphenylphosphine)palladium(0) or bis(triphenylphosphine)palladium(II) dichloride, and a base such as potassium carbonate or sodium carbonate, in a suitable solvent such as aqueous dioxane or aqueous tetrahydrofuran, at an appropriate temperature, preferably between 80-120° C. The cross-coupling between boronate Intermediate (1 g) and halide Intermediate (1 h), to afford Intermediate (1c) may be effected using a catalyst such as bis(triphenylphosphine)palladium(II) dichloride or [1,1′-bis(diphenylphosphino)ferrocene]dichloro-palladium(II) and a base such as potassium fluoride, potassium carbonate, or sodium bicarbonate, in a suitable solvent such as aqueous dioxane or aqueous 1,2-dimethoxyethane, at an appropriate temperature, preferably between 80-110° C.

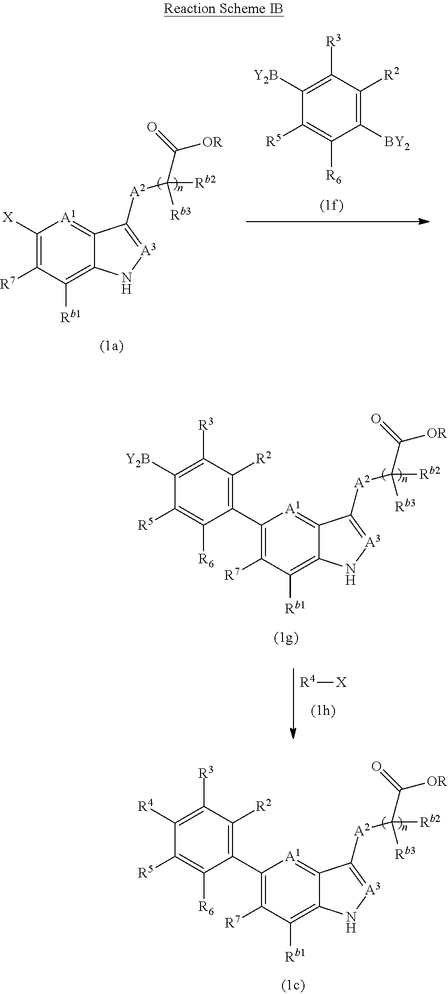

Reaction Scheme II outlines general procedures for the synthesis of Intermediates (2d). In Reaction Scheme II, the chemical groups are as defined in the Specification; additionally in Reaction Scheme II: X=a halide suitable for transition metal catalyzed cross-coupling reactions, preferably iodo, bromo, or chloro; R=a carbon-linked (hetero)alkyl or (hetero)aryl group where —C(O)OR is an ester that is chemically inert to the described reaction cross-coupling reactions but where —C(O)OR may be readily converted to —C(O)OR¹ to provide compounds of Formula (I), preferably R=methyl, ethyl, benzyl, or tert-butyl.

Substituted (aza)indole Intermediates (2a) can be prepared based on methods reported in the literature (RSC Advances 2014, 4, 4672; WO2014049133; European Journal of Organic Chemistry 2008, 5, 783; WO2019236957; Journal of Medicinal Chemistry 2021, 64, 14968; RSC Advances 2017, 7, 52852; US20190185469; WO2013010880). Intermediates (2b) can be synthesized from (aza)indole Intermediates (2a) based on literature procedures (RSC Advances 2018, 8, 13121; WO2011123946; WO2020173400). Unsaturated Intermediates (2c) can be prepared by olefination of aldehyde Intermediates (2b) under standard conditions (WO200979767; Chemical Reviews 1989, 89, 863; Organic Letters 2017, 19, 1500; European Journal of Medicinal Chemistry 2022, 234, 114248; European Journal of Medicinal Chemistry 2010, 45, 298; WO2016102633). Intermediates (2d) can be prepared by reduction of unsaturated Intermediates (2c) with appropriate choice of reaction conditions to avoid undesired reduction of other functional groups; hydrogenation with a platinum catalyst can promote selective olefin reduction in the presence of halides (Journal of the American Chemical Society 1922, 44, 1397; Journal of the American Chemical Society 1960, 82, 6090; WO200876805; US2020247768). Alternatively, Intermediates (2d) can be prepared directly from Intermediates (2b) by reaction with malonate derivatives (Synthetic Communications 25, 3067; U.S. Pat. No. 5,350,872; EP3275867; WO201535223; Bioorganic & Medicinal Chemistry 2017, 25, 2995).

Aldehyde Intermediate (2b) may be prepared from fused pyrrole Intermediate (2a) by reaction with sodium nitrite and an acid, preferably hydrochloric acid, in a suitable solvent, such as aqueous N,N-dimethylformamide, aqueous dioxane, aqueous acetone, or water, at an appropriate temperature, preferably between 0-30° C. Olefin Intermediate (2c) may be synthesized from aldehyde Intermediate (2b) by reaction with an appropriate olefinating reagent, preferably (carbethoxymethylene)triphenylphosphorane or (tert-butoxycarbonylmethylene)triphenylphosphorane, in an appropriate solvent such as tetrahydrofuran, dichloromethane, or ethanol, at an appropriate temperature, preferably between 20-70° C. Reduction of olefin Intermediate (2c) to Intermediate (2d) may be achieved by catalytic hydrogenation, preferably using platinum oxide as catalyst and hydrogen gas as reductant, in a suitable solvent such as ethanol or ethyl acetate, at an appropriate temperature, preferably between 20-40° C. Alternatively, Intermediate (2d) may be prepared directly from aldehyde Intermediate (2b) by reaction with a malonate derivative, preferably 2,2-dimethyl-1,3-dioxane-4,6-dione, and a base and a reducing agent, preferably triethylamine and formic acid, in an appropriate solvent, preferably dioxane, at an appropriate temperature, preferably between 50-100° C.

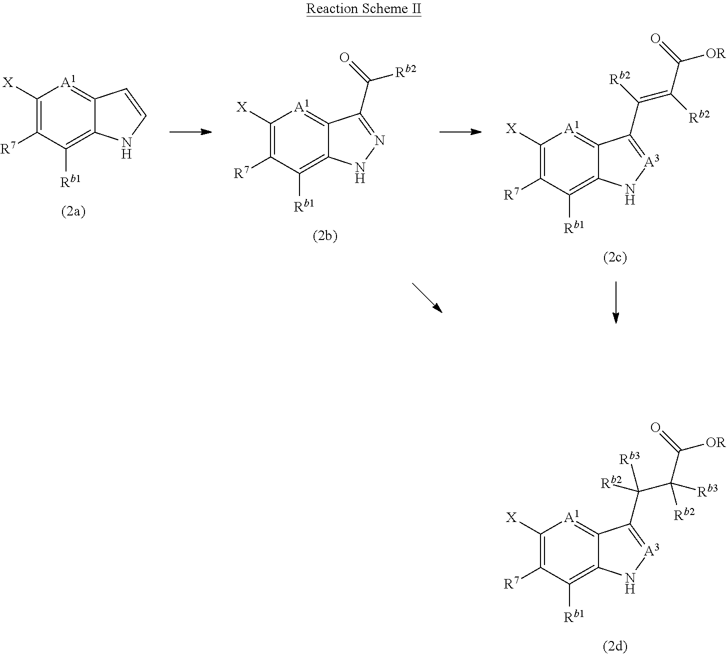

Reaction Scheme III outlines general procedures for the synthesis of Intermediates (3g) via standard protecting group and functional group interconversions that are well known to those skilled in the art. In Reaction Scheme III, the chemical groups are as defined in the Specification; except that in Reaction Scheme III, A² is limited to CH₂, CHD, CD₂; additionally in Reaction Scheme III: X=a halide suitable for transition metal catalyzed cross-coupling reactions, preferably iodo, bromo, or chloro; R=a carbon-linked (hetero)alkyl or (hetero)aryl group where —C(O)OR is an ester that is chemically inert to the described reaction cross-coupling reactions but where —C(O)OR may be readily converted to —C(O)OR¹ to provide compounds of Formula (I), preferably R=methyl, ethyl, benzyl, or tert-butyl; PG=a protecting group suitable for the reactions described, preferably 2-tetrahydropyranyl or (2-(trimethylsilyl)ethoxy)methyl; X¹=a leaving group suitable for the reactions described, preferably mesylate, tosylate, chloride, or bromide; m=0 or 1.

N-protected Intermediates (3b) can be prepared from Intermediates (3a) using standard protecting group strategies as described in P. G. M. Wuts, Greene's Protective Groups in Organic Synthesis, John Wiley & Sons, New York, 2014. General approaches for homologation of carboxylic acid derivatives are described in Synthesis 1979, 1979, 633. Carboxylic acid Intermediate (3c) can be synthesized by hydrolysis or deprotection of ester Intermediate (3b) (WO2012137089; Tetrahedron Letters 2018, 59, 2917; WO2022150574; WO2022229341). Alcohol Intermediate (3d) can be prepared by reduction of carboxylic acid Intermediate (3c) using hydride sources as described in Advanced Synthesis & Catalysis 2021, 363, 4867; Journal of Medicinal Chemistry 2011, 54, 1333. Alcohol Intermediate (3d) can be converted to leaving group Intermediate (3e) as described in Synthesis 2006, 10, 1635; Journal of the American Chemical Society 2020, 142, 2766; Chemical Communications 2018, 54, 1877. Nitrile Intermediate (3f) can be prepared from Intermediate (3e) by reaction with a cyanide source (Organic Letters 2017, 19, 4742; WO2022204150). Alternatively, nitrile Intermediate (3f) can be prepared directly from alcohol Intermediate (3d) (Tetrahedron Letters 1999, 40, 7355). Ester Intermediate (3g) can be synthesized from nitrile Intermediate (3f) by treatment with alcoholic acid both to convert the nitrile and to cleave an acid-labile N-protecting group (Synthetic Communications 2003, 33, 3271; European Journal of Organic Chemistry 2000, 21, 3575).

N-Protected Intermediate (3b) may be prepared from Intermediate (3a) by reaction with an appropriate protecting group reagent and an acid or base as appropriate. Preferably the 2-tetrahydropyranyl protecting group may be introduced using 3,4-dihydro-2H-pyran and p-toluenesulfonic acid in a solvent such as THF, at an appropriate temperature, preferably between 20-50° C. Alternatively, the (2-(trimethylsilyl)ethoxy)methyl protecting group may be introduced using (2-(trimethylsilyl)ethoxy)methyl chloride and an appropriate base, such as sodium hydride, potassium tert-butoxide, or N,N-diisopropylethylamine, in a solvent such as tetrahydrofuran or N,N-dimethylformamide, at an appropriate temperature, preferably between 0-30° C. The conversion of ester Intermediate (3b) to acid Intermediate (3c) may be effected under standard conditions for cleavage of the —C(O)OR ester. For R=small alkyl, such as methyl or ethyl, hydrolysis to afford carboxylic acid Intermediate (3c) may be effected with a suitable metal hydroxide, such as sodium hydroxide, potassium hydroxide, or lithium hydroxide, in a solvent such as water, or a mixed aqueous-organic solvent such as aqueous methanol, ethanol, and/or tetrahydrofuran at an appropriate temperature, preferably between 20-60° C. For R=tert-butyl, acid-promoted cleavage of the tert-butyl ester to afford carboxylic acid Intermediate (3c) may be effected with a suitable acid and solvent combination, such as trifluoroacetic acid in dichloromethane, or hydrogen chloride in dioxane, at an appropriate temperature, preferably between 0-30° C.

Reduction of acid Intermediate (3c) to alcohol Intermediate (3d) may be effected with an appropriate hydride source, preferably borane, borane tetrahydrofuran complex, or lithium aluminum hydride in a solvent such as tetrahydrofuran or diethyl ether, at an appropriate temperature, preferably between 0-60° C. Leaving group Intermediate (3e) may be prepared from alcohol Intermediate (3d) by reaction with an activating reagent and a base, preferably methanesulfonic anhydride or methanesulfonyl chloride and triethylamine or pyridine, in a suitable solvent such as dichloromethane, at an appropriate temperature, preferably between 0-30° C. Nitrile Intermediate (3f) may be prepared from leaving group Intermediate (3e) by reaction with a cyanide source in an appropriate solvent, such as trimethylsilyl cyanide and potassium fluoride in N,N-dimethylformamide, or sodium cyanide in dimethylsulfoxide, at an appropriate temperature, preferably between 25-90° C. Ester Intermediate (3g) may be synthesized by treating nitrile Intermediate (3f) with alcoholic acid, preferably sulfuric acid or hydrogen chloride in methanol or ethanol; the acidic reaction conditions also cleave the acid-labile N-protecting group.

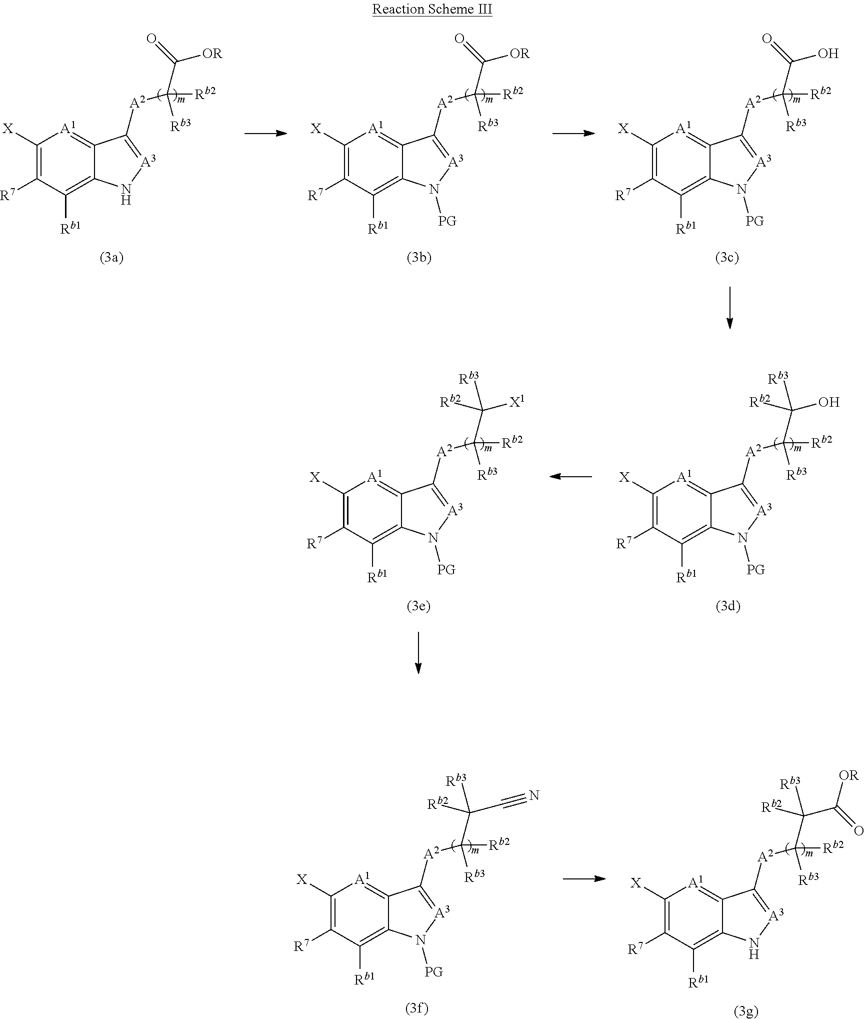

Reaction Scheme IV outlines general procedures for the synthesis of compounds of Formula (I). In Reaction Scheme IV, the chemical groups are as defined in the Specification; except that in Reaction Scheme IV, A² is limited to S, O, NH; additionally in Reaction Scheme IV: X=a halide suitable for transition metal catalyzed cross-coupling reactions, preferably bromo or chloro; R=a carbon-linked (hetero)alkyl or (hetero)aryl group where —C(O)OR is an ester that is chemically inert to the described reaction cross-coupling reactions but where —C(O)OR may be readily converted to —C(O)OR¹ to provide compounds of Formula (I), preferably R=methyl, ethyl, benzyl, or tert-butyl; —BY₂=a boronic acid or boronate ester suitable for transition metal catalyzed cross-coupling reactions, preferably boronic acid or pinacolboronate; PG=a protecting group suitable for the reactions described, preferably 2-tetrahydropyranyl.

Substituted (aza)indazole intermediates (4a) are commercially available and are well-known in the literature, as is their conversion to 3-iodo Intermediates (4b) and then to N-protected Intermediates (4c) (European Journal of Medicinal Chemistry 2020, 203, 11255; Synlett 2009, 615-619; Journal of Organic Chemistry 2009, 74, 6331; WO2023196720; WO2023091707; WO2022133037; WO2013030138; WO2018011628; Journal of Medicinal Chemistry 2017, 60, 2361; P. G. M. Wuts, Greene's Protective Groups in Organic Synthesis, John Wiley & Sons, New York, 2014). Intermediates (4e) can be prepared by transition metal catalyzed cross-coupling of iodide Intermediates (4c) and thiol Intermediate (4d) (A²=S; WO2009149837; WO2011138265), or alcohol Intermediate (4d) (A²=0; WO2009089359; WO2016004272; WO2009149837), or amine Intermediate (4d) (A²=NH; European Journal of Medicinal Chemistry 2021, 213, 113192; Synthesis 2011, 16, 2651). Cross coupling product Intermediates (4f) can be prepared from halide Intermediates (4e) and boronate Intermediates (1b) by methods analogous to those described in Reaction Scheme IA for the conversion of Intermediates (1a) to Intermediates (1c). Compounds of Formula (I) can be prepared from Intermediates (4f) by sequential cleavage of the N-protecting group to Intermediate (4g) followed by ester hydrolysis, or by sequential ester hydrolysis to Intermediate (4h) followed by cleavage of the N-protecting group. Hydrolysis of Intermediates (4f) or Intermediates (4g) can be conducted based upon procedures well known in the literature (WO2012137089; Tetrahedron Letters 2018, 59, 2917; WO2022150574; WO2022229341). Cleavage of N-protecting groups from Intermediates (4f) or Intermediates (4h) can be conducted based on P. G. M. Wuts, Greene's Protective Groups in Organic Synthesis, John Wiley & Sons, New York, 2014.

Intermediate (4b) can be prepared by iodination of Intermediate (4a) by reaction with an appropriate iodinating reagent such as iodine or N-iodosuccinimide, in the presence of a base such as potassium hydroxide or potassium tert-butoxide, in an appropriate solvent such as N,N-dimethylformamide or tetrahydrofuran, at an appropriate temperature, preferably between 0-30° C. N-Protected Intermediate (4c) may be prepared from Intermediate (4b) by reaction with an appropriate protecting group reagent and an acid or base as appropriate. Preferably the 2-tetrahydropyranyl protecting group may be introduced using 3,4-dihydro-2H-pyran and p-toluenesulfonic acid in a solvent such as tetrahydrofuran, at an appropriate temperature, preferably between 20-50° C. Alternatively, the (2-(trimethylsilyl)ethoxy)methyl protecting group may be introduced using 2-(trimethylsilyl)ethoxymethyl chloride and an appropriate base, such as sodium hydride, potassium tert-butoxide, or N,N-diisopropylethylamine, in a solvent such as tetrahydrofuran or N,N-dimethylformamide at an appropriate temperature, preferably between 0-30° C. Intermediate (4e) can be prepared by transition metal catalyzed cross-coupling of iodide Intermediate (4c) and thiol Intermediate (4d) (A²=S) using a transition metal catalyst and a ligand, preferably Pd₂(dba)₃ and Xantphos, in an appropriate solvent such as toluene, with an appropriate base, preferably N,N-diisopropylethylamine, at an appropriate temperature, preferably between 40-70° C.

Intermediates (4e) can be prepared by transition metal catalyzed cross-coupling of iodide Intermediates (4c) and alcohol Intermediate (4d) (A²═O) using a transition metal catalyst and a ligand, preferably copper(I)iodide and 1,10-phenanthroline, in an appropriate solvent such as toluene or ethanol, with an appropriate base, preferably potassium fluoride or cesium carbonate, at an appropriate temperature, preferably between 100-120° C. Intermediate (4e) can be prepared by transition metal catalyzed cross-coupling of iodide Intermediate (4c) and amine Intermediate (4d) (A²=NH) using a transition metal catalyst and a ligand, preferably copper(I)iodide and N-(2,6-difluorophenyl)-6-hydroxypicolinamide, in an appropriate solvent such as dioxane or toluene, with an appropriate base and additive, preferably potassium carbonate and sodium ascorbate, at an appropriate temperature, preferably between 90-110° C. The cross-coupling between halide Intermediate (4e) and boronate Intermediate (1b) may be effected using a catalyst such as bis(triphenylphosphine)palladium(II) dichloride or [1,1′-bis(diphenylphosphino)ferrocene] dichloro-palladium(II) and a base such as potassium fluoride, potassium carbonate, or sodium bicarbonate, in a suitable solvent such as aqueous dioxane or aqueous 1,2-dimethoxyethane, at an appropriate temperature, preferably between 80-110° C.

The conversion of ester Intermediate (4f) to Intermediate (4h), or of ester Intermediate (4g) to a compound of Formula (I), may be effected under standard conditions for cleavage of the —C(O)OR ester. For R=small alkyl, such as methyl or ethyl, hydrolysis to afford a carboxylic acid may be effected with a suitable metal hydroxide, such as sodium hydroxide, potassium hydroxide, or lithium hydroxide, in a solvent such as water, or a mixed aqueous-organic solvent such as aqueous methanol, ethanol, and/or tetrahydrofuran at an appropriate temperature, preferably between 0-110° C. For R=tert-butyl, acid-promoted cleavage of the tert-butyl ester to afford a carboxylic acid may be effected with a suitable acid and solvent combination, such as trifluoroacetic acid in dichloromethane, or hydrogen chloride in dioxane, at an appropriate temperature, preferably between 0-30° C. The 2-tetrahydropyranyl protecting group may be removed by treatment of Intermediate (4f) or Intermediate (4h) with an appropriate acid, preferably trifluoroacetic acid, in an appropriate solvent such as dichloromethane, at an appropriate temperature, preferably between 10-30° C. The (2-(trimethylsilyl)ethoxy)methyl protecting group may be removed by treatment of Intermediate (4f) or Intermediate (4h) with an appropriate acid-solvent combination, such as trifluoroacetic acid-dichloromethane, or hydrochloric acid-methanol, at an appropriate temperature, preferably between 10-50° C. Alternatively, the (2-(trimethylsilyl)ethoxy)methyl protecting group may be removed by treatment of Intermediate (4f) or Intermediate (4h) with an appropriate fluoride source, such as tetra-N-butylammonium fluoride, in an appropriate solvent, such as tetrahydrofuran, at an appropriate temperature, preferably between 20-60° C.

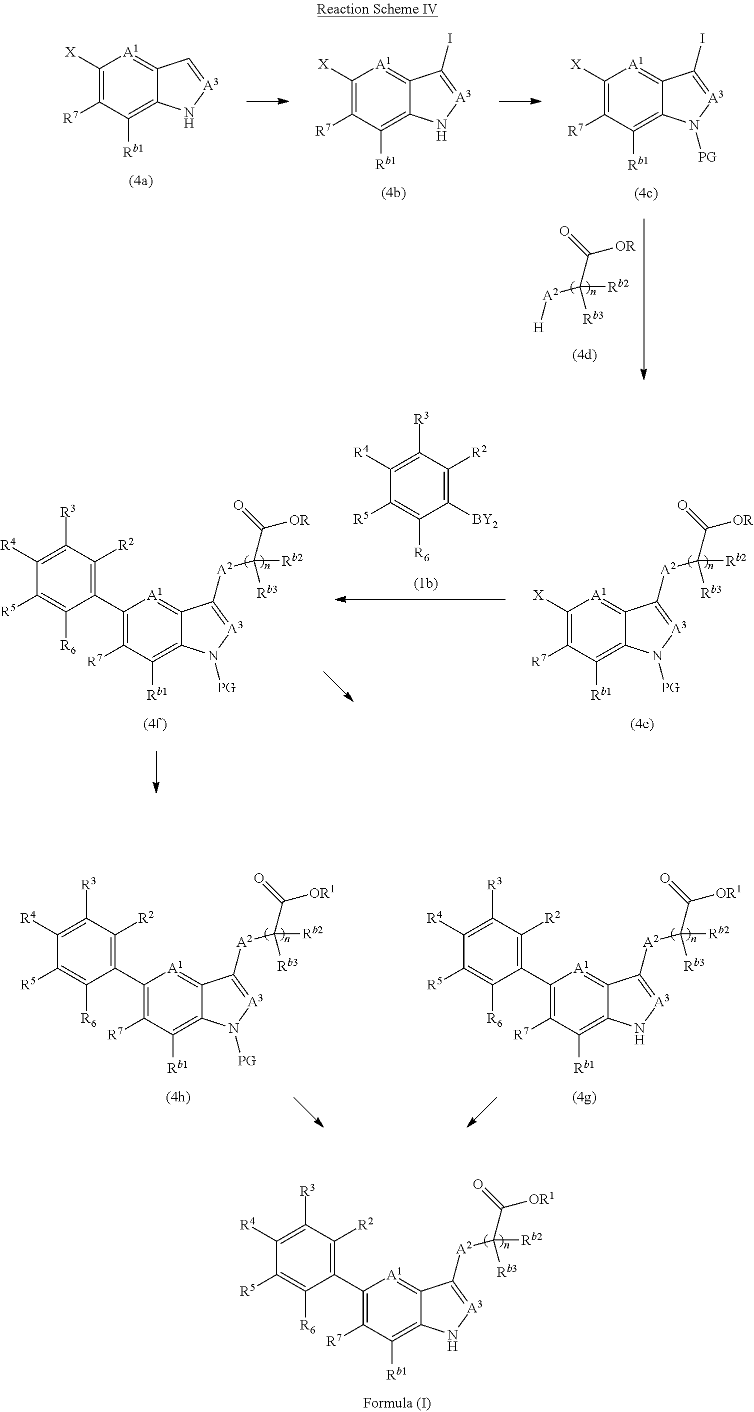

### EXAMPLES

In order that this invention may be better understood, the following examples are set forth. These examples are for purposes of illustration only and are not to be construed as limiting the scope of the invention in any manner.

### Liquid Chromatography Analytical Methods

LC Method A: Acquity UPLC BEH C18 2.1 mm×50 mm, 1.7μ; A: 10 mM ammonium acetate in 95:5 H₂O:CH₃CN, B: 10 mM ammonium acetate in 5:95 H₂O:CH₃CN, Gradient 5%-100% B over 1 min, then 100% B for 0.2 min; 1.0 mL/min.

LC Method B: Xbridge C18 2.1 mm×50 mm, 5μ; A: 0.0375% TFA in H₂O, B: 0.01875% TFA in CH₃CN, Gradient 1%-5% B over 0.6 min, then to 100% B over 3.4 min; 0.8 mL/min; 40° C.

LC Method C: Xbridge C18 2.1 mm×50 mm, 5μ; A: 0.0375% TFA in H₂O, B: 0.01875% TFA in CH₃CN, Gradient 10% B for 0.5 min, then to 100% B over 3.5 min; 0.8 mL/min, 40° C.

LC Method D: Xbridge C18 2.1 mm×50 mm, 5μ; A: 0.0375% TFA in H₂O, B: 0.01875% TFA in CH₃CN, Gradient 25% B for 0.5 min, then to 100% B over 3.0 min; 0.8 mL/min, 40° C.

LC Method E: Xbridge C18 2.1 mm×50 mm, 5μ; A: 0.05% NH₄OH in H₂O, B: CH₃CN, Gradient 5% B for 0.5 min, then to 100% B over 2.9 min; 0.8 mL/min, 40° C.

LC Method F: Xbridge C18 2.1 mm×50 mm, 5μ; A: 0.05% NH₄OH in H₂O, B: CH₃CN, Gradient 5% B for 0.5 min, then to 100% B over 2.9 min; 0.8 mL/min, 60° C.

LC Method G: Waters Atlantis C18 4.6×50 mm, 5μ; A: 0.05% TFA in H₂O, B: 0.05% TFA in CH₃CN, Gradient 5-95% B over 4 min; 2 mL/min.

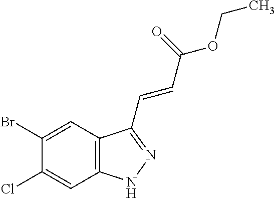 [SMILES: CCOC(=O)/C=C/c1n[nH]c2cc(Cl)c(Br)cc12]

### Preparation 1. Ethyl (E)-3-(5-bromo-6-chloro-1H-indazol-3-yl)acrylate

(Carbethoxymethylene)triphenylphosphorane (50.3 g, 145 mmol) was added to a solution of 5-bromo-6-chloro-1H-indazole-3-carbaldehyde (25.0 g, 96.0 mol) in THF (500 mL). The resulting mixture was heated at 50° C. for 16 h, then was concentrated. The residue was purified by silica gel chromatography (0-20% THF:petroleum ether) to afford ethyl (E)-3-(5-bromo-6-chloro-1H-indazol-3-yl)acrylate as a yellow solid (23 g).

¹H NMR (400 MHz, DMSO-d₆) δ 13.86 (br s, 1H), 8.64 (s, 1H), 7.92 (s, 1H), 7.87 (d, 1H), 6.82 (d, 1H), 4.22 (q, 2H), 1.28 (t, 3H); MS (M+H)⁺ 330.9.

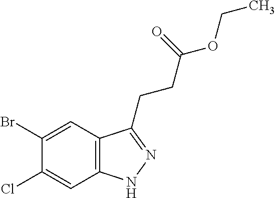 [SMILES: CCOC(=O)CCc1n[nH]c2cc(Cl)c(Br)cc12]

### Preparation 2. Ethyl 3-(5-bromo-6-chloro-1H-indazol-3-yl)propanoate

PtO₂ (3.17 g, 14.0 mmol) was added to a solution of ethyl (E)-3-(5-bromo-6-chloro-1H-indazol-3-yl)acrylate (23 g, 70 mmol) in EtOH (1.0 L) and EtOAc (0.30 L). The mixture was stirred at 25° C. under an atmosphere of H₂ gas (balloon) for 16 h. Filtration and concentration afforded the crude product which was purified by silica gel chromatography (0-20% THF:petroleum ether) to afford ethyl 3-(5-bromo-6-chloro-1H-indazol-3-yl)propanoate as a white solid (16 g).

¹H NMR (DMSO-d₆) δ 12.99 (s, 1H), 8.25 (s, 1H), 7.77 (s, 1H), 4.03 (q, 2H), 3.16 (t, 2H), 2.78 (t, 2H), 1.14 (t, 3H); MS (M+H)+332.9.

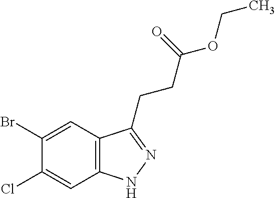 [SMILES: CCOC(=O)CCc1n[nH]c2cc(Cl)c(Br)cc12]

### Preparation 2, Alternate Procedure. Ethyl 3-(5-bromo-6-chloro-1H-indazol-3-yl)propanoate

2,2-Dimethyl-1,3-dioxane-4,6-dione (183 g, 1.27 mmol), Et₃N (322 mL, 2.31 mol), and formic acid (214 mL, 5.67 mol) were added sequentially to a solution of 5-bromo-6-chloro-1H-indazole-3-carbaldehyde (300 g, 1.16 mol) in dioxane (3.0 L). The resulting mixture was heated at 100° C. for 16 h, then was cooled to ambient temperature and H₂O (200 mL) was added. Aqueous HCl solution (3 M) was added to adjust pH to ˜2-3, then the solution was extracted with EtOAc (2×100 mL). The combined organics were washed with saturated aqueous NaCl solution (3×100 mL), dried over MgSO₄, filtered, and concentrated. The resulting residue was purified by silica gel chromatography (0-5% THF:DCM) to afford ethyl 3-(5-bromo-6-chloro-1H-indazol-3-yl)propanoate as a yellow solid. Two reactions on this same scale provided a combined total of 469 g of ethyl 3-(5-bromo-6-chloro-1H-indazol-3-yl)propanoate.

¹H NMR (DMSO-d₆) δ 12.99 (s, 1H), 8.25 (s, 1H), 7.77 (s, 1H), 4.03 (q, 2H), 3.16 (t, 2H), 2.78 (t, 2H), 1.14 (t, 3H); MS (M+H)⁺ 332.9.

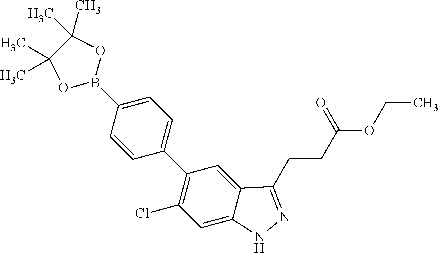 [SMILES: CCOC(=O)CCc1n[nH]c2cc(Cl)c(-c3ccc(B4OC(C)(C)C(C)(C)O4)cc3)cc12]

### Preparation 3. Ethyl 3-(6-chloro-5-(4-(4,4,5,5-tetramethyl-1,3,2-dioxaborolan-2-yl)phenyl)-1H-indazol-3-yl)propanoate

1,4-Bis(4,4,5,5-tetramethyl-1,3,2-dioxaborolan-2-yl)benzene (35.8 g, 109 mmol), Pd(PPh₃)₄(2.51 g, 2.17 mmol), and an aqueous solution of K₂CO₃ (0.2 M, 72.4 mL, 145 mmol) were added sequentially to a solution of ethyl 3-(5-bromo-6-chloro-1H-indazol-3-yl)propanoate (12 g, 36 mmol) in dioxane (150 mL). The resulting mixture was stirred under N₂ gas at 120° C. for 5 h, then was cooled and was diluted with H₂O (50 mL). The volatile organics were evaporated under reduced pressure, then the residue was extracted with EtOAc (2×100 mL). The combined organics were dried over Na₂SO₄, filtered, and concentrated. The resulting residue was purified by silica gel chromatography (0-20% THF:petroleum ether) to afford a solid which was slurried in petroleum ether (50 mL). Filtration and drying afforded ethyl 3-(6-chloro-5-(4-(4,4,5,5-tetramethyl-1,3,2-dioxaborolan-2-yl)phenyl)-1H-indazol-3-yl)propanoate as a yellow solid (7.0 g).

¹H NMR (400 MHz, DMSO-d₆) δ 12.89 (s, 1H), 7.76 (m, 3H), 7.68 (s, 1H), 7.47 (d, 2H), 4.03 (q, 2H), 3.18 (t, 2H), 2.79 (t, 2H), 1.33 (s, 12H), 1.12 (t, 3H); MS (M+H)⁺ 455.1.

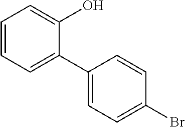 [SMILES: Oc1ccccc1-c1ccc(Br)cc1]

### Preparation 4. 4′-Bromo-[1,1′-biphenyl]-2-ol

(2-Hydroxyphenyl)boronic acid (536 g, 1.17 mol), Pd(PPh₃)₄(102 g, 26.5 mmol), and K₃PO₄ (1125 g, 1.59 mol) were added sequentially to a solution of 1-bromo-4-iodobenzene (1000 g, 1.06 mol) in H₂O (2.5 L) and dioxane (7.5 L). The mixture was stirred under N₂ gas at 80° C. for 17 h, then was cooled and was partitioned between H₂O (1.0 L) and EtOAc (3×1.0 L). The combined organics were washed with saturated aqueous NaCl solution (1.0 L), dried over MgSO₄, filtered, and concentrated. The resulting residue was purified by silica gel chromatography (0-7% EtOAc:petroleum ether) to afford 4′-bromo-[1,1′-biphenyl]-2-ol as a yellow oil (625 g).

¹H NMR (400 MHz, DMSO-d₆) δ 9.63 (s, 1H), 7.58 (d, 2H), 7.50 (d, 2H), 7.25 (d, 1H), 7.17 (m, 1H), 6.95 (dd, 1H), 6.87 (m, 1H).

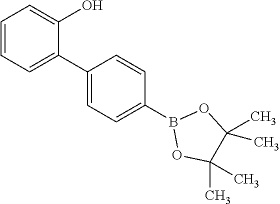 [SMILES: CC1(C)OB(c2ccc(-c3ccccc3O)cc2)OC1(C)C]

### Preparation 5. 4′-(4,4,5,5-Tetramethyl-1,3,2-dioxaborolan-2-yl)-[1,1′-biphenyl]-2-ol

B₂Pin₂ (808 g, 1.16 mol), Pd(dppf)Cl₂ (91.8 g, 48.1 mmol), and KOAc (369 g, 1.45 mol) were added sequentially to a solution of 4′-bromo-[1,1′-biphenyl]-2-ol (625 g, 963 mmol) in dioxane (6.5 L), and the resulting mixture was stirred under N₂ gas at 100° C. for 16 h, then was cooled and was partitioned between H₂O (1.5 L) and EtOAc (3×1.5 L). The combined organics were washed with saturated aqueous NaCl solution (1.0 L), dried over MgSO₄, filtered, and concentrated. The resulting residue was purified by silica gel chromatography (0-3% EtOAc:petroleum ether) to afford 4′-(4,4,5,5-tetramethyl-1,3,2-dioxaborolan-2-yl)-[1,1′-biphenyl]-2-ol as a white solid (500 g).

¹H NMR (400 MHz, CDC₃) δ 7.86 (d, 2H), 7.41 (d, 2H), 7.18 (m, 2H), 6.91 (m, 2H), 5.16 (s, 1H), 1.29 (s, 12H).

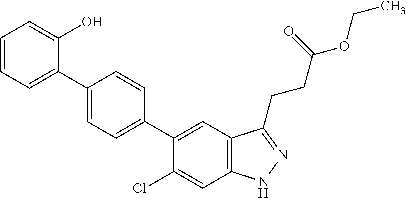 [SMILES: CCOC(=O)CCc1n[nH]c2cc(Cl)c(-c3ccc(-c4ccccc4O)cc3)cc12]

### Preparation 6. Ethyl 3-(6-chloro-5-(2′-hydroxy-[1,1′-biphenyl]-4-yl)-1H-indazol-3-yl)propanoate

A solution of ethyl 3-(5-bromo-6-chloro-1H-indazol-3-yl)propanoate (5.00 g, 15.0 mmol), 4′-(4,4,5,5-tetramethyl-1,3,2-dioxaborolan-2-yl)-[1,1′-biphenyl]-2-ol (5.36 g, 18.1 mmol), and KF (2.68 g, 45.2 mmol) in dioxane (75 mL) and H₂O (25 mL) was sparged with N₂ gas for 20 min. Pd(dppf)Cl₂ (1.10 g, 1.51 mmol) was added and the mixture was heated to 80° C. for 17 h, then was cooled to ambient temperature. The mixture was partitioned between EtOAc (2×) and H₂O, and the combined organics were dried over MgSO₄, filtered, and concentrated. The residue was purified via silica gel chromatography (20-50% EtOAc:heptane) to afford a solid which was slurried in EtOH. Filtration and drying afforded ethyl 3-(6-chloro-5-(2′-hydroxy-[1,1′-biphenyl]-4-yl)-1H-indazol-3-yl)propanoate as a white solid (2.6 g).

¹H NMR (400 MHz, DMSO-d₆) δ 12.85 (s, 1H), 9.58 (s, 1H), 7.83 (s, 1H), 7.68 (s, 1H), 7.64 (d, 2H), 7.48 (d, 2H), 7.33 (dd, 1H), 7.18 (m, 1H), 6.97 (d, 1H), 6.91 (m, 1H), 4.03 (q, 2H), 3.19 (t, 2H), 2.79 (t, 2H), 1.13 (t, 3H); MS (M+H)*421.2.

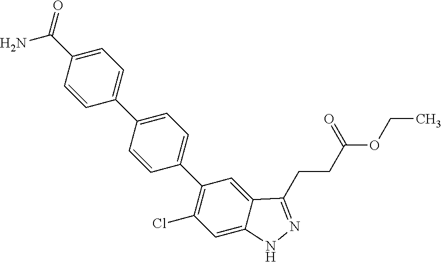 [SMILES: CCOC(=O)CCc1n[nH]c2cc(Cl)c(-c3ccc(-c4ccc(C(N)=O)cc4)cc3)cc12]

### Preparation 7. Ethyl 3-(5-(4′-carbamoyl-[1,1′-biphenyl]-4-yl)-6-chloro-1H-indazol-3-yl)propanoate

Pd(dppf)Cl₂—CH₂Cl₂ (19 mg, 0.026 mmol) was added to a mixture of 4-bromobenzamide (58 mg, 0.29 mmol), ethyl 3-(6-chloro-5-(4-(4,4,5,5-tetramethyl-1,3,2-dioxaborolan-2-yl)phenyl)-1H-indazol-3-yl)propanoate (120 mg, 0.26 mmol), and K₂CO₃ (109 mg, 0.79 mmol) in dioxane (5 mL) and H₂O (1 mL) and the resulting mixture was heated at 80° C. for 2 h. The mixture was cooled to ambient temperature and then was extracted with EtOAc (2×20 mL). The combined organics were dried over Na₂SO₄, filtered, and concentrated. The resulting solid was purified via silica gel chromatography (50-100% petroleum ether:EtOAc, then 10:1 EtOAc:MeOH) to afford ethyl 3-(5-(4′-carbamoyl-[1,1′-biphenyl]-4-yl)-6-chloro-1H-indazol-3-yl)propanoate as a yellow solid (95 mg). MS (M+H)⁺ 448.1.

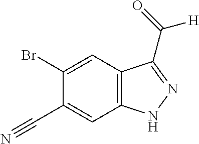 [SMILES: N#Cc1cc2[nH]nc(C=O)c2cc1Br]

### Preparation 8. 5-Bromo-3-formyl-1H-indazole-6-carbonitrile

A solution of NaNO₂ (521 mg, 7.6 mmol) in H₂O (3.8 mL) was added to a solution of 5-bromo-1H-indole-6-carbonitrile (167 mg, 0.76 mmol) in acetone (7.6 mL). The mixture was sparged with N₂ for 0.5 min, then was cooled to 0° C. Hydrochloric acid (2 M, 3.8 mL, 7.6 mmol) was added and the resulting mixture was stirred at 0° C. for 1.5 h, then at ambient temperature for 3.5 h. The resulting solids (120 mg), a mixture of 5-bromo-3-formyl-1H-indazole-6-carbonitrile and unreacted 5-bromo-1H-indole-6-carbonitrile, were collected and were used in the next step without further purification. MS (M−H)⁻ 248.1.

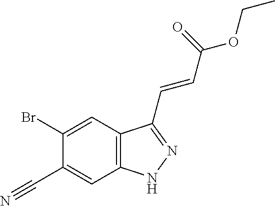 [SMILES: CCOC(=O)/C=C/c1n[nH]c2cc(C#N)c(Br)cc12]

### Preparation 9. Ethyl (E)-3-(5-bromo-6-cyano-1H-indazol-3-yl)acrylate

(Carbethoxymethylene)triphenylphosphorane (209 mg, 0.60 mmol) was added to a solution of 5-bromo-3-formyl-1H-indazole-6-carbonitrile (100 mg, 0.40 mmol) in THF (1.5 mL) and the resulting mixture was heated at 50° C. for 5 h. The mixture was concentrated and the residue was purified by silica gel chromatography (5-65% EtOAc:heptane) to afford ethyl (E)-3-(5-bromo-6-cyano-1H-indazol-3-yl)acrylate, contaminated with 5-bromo-1H-indole-6-carbonitrile. The material was used in the next reaction without further purification. MS (M−H)⁻ 318.1.

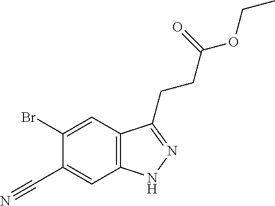 [SMILES: CCOC(=O)CCc1n[nH]c2cc(C#N)c(Br)cc12]

### Preparation 10. Ethyl 3-(5-bromo-6-cyano-1H-indazol-3-yl)propanoate

A suspension of ethyl 3-(5-bromo-6-cyano-1H-indazol-3-yl)acrylate (47 mg, 0.15 mmol) in EtOH (15 mL) was added to PtO₂ (13 mg, 0.057 mmol) and EtOH (2 mL) in a Paar reactor. The reactor was purged sequentially with N₂ (50 psi, 3×) and with H₂ (30 psi, 3×) and then was held under H₂ for 13 h. The resulting mixture was filtered through Celite, rinsing with EtOH, and the filtrate was concentrated. Silica gel chromatography (0-70% EtOAc:heptane) afforded ethyl 3-(5-bromo-6-cyano-1H-indazol-3-yl)propanoate (18 mg). MS (M−H)⁻ 320.1.

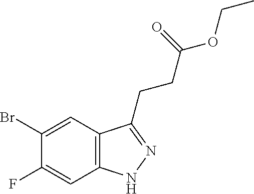 [SMILES: CCOC(=O)CCc1n[nH]c2cc(F)c(Br)cc12]

### Preparation 11. Ethyl 3-(5-bromo-6-fluoro-1H-indazol-3-yl)propanoate

Ethyl 3-(5-bromo-6-fluoro-1H-indazol-3-yl)propanoate was prepared from 5-bromo-6-fluoro-1H-indazole-3-carbaldehyde by analogous methods to Preparations 9 and 10. MS (M−H)⁻ 313.2.

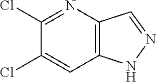 [SMILES: Clc1cc2[nH]ncc2nc1Cl]

### Preparation 12. 5,6-Dichloro-1H-pyrazolo[4,3-b]pyridine

KOAc (2.36 g, 24 mmol) and Ac₂O (7.56 mL, 80 mmol) were added sequentially to a solution of 5,6-dichloro-2-methylpyridin-3-amine (3.5 g, 20 mmol) in CHCl₃ (10 mL). The resulting mixture was heated at 60° C. for 2 h, then dicyclohexano-18-crown-6 (745 mg, 2.0 mmol) and a solution of isoamyl nitrite (6.45 mL, 47 mmol) in CHCl₃ (5 mL) were added and heating at 60° C. was continued for 21 h. The mixture was cooled and was concentrated. The resulting residue was dissolved in MeOH (50 mL) and H₂O (11 mL), then solid K₂CO₃ was added at 0° C., and the mixture was stirred for 10 min at 0° C., then for 1.5 h at ambient temperature. The resulting solids were collected by filtration to afford 5,6-dichloro-1H-pyrazolo[4,3-b]pyridine (1.9 g). MS (M−H)⁻ 186.1.

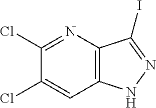 [SMILES: Clc1cc2[nH]nc(I)c2nc1Cl]

### Preparation 13. 5,6-Dichloro-3-iodo-1H-pyrazolo[4,3-b]pyridine

Solid KOH (673 mg, 12 mmol) and 12 (1.83 g, 7.2 mmol) were sequentially added to a solution of 5,6-dichloro-1H-pyrazolo[4,3-b]pyridine (752 mg, 4.0 mmol) in DMF (15 mL) at 0° C. The resulting mixture was stirred at 0° C. for 30 min, then at ambient temperature for 18 h. Excess 12 was quenched by addition of saturated aqueous Na₂S2O₃ solution, then the mixture was diluted with H₂O (30 mL) and was acidified to pH 3-4 by addition of aqueous HCl solution (1 M, 6 mL). The mixture was extracted with 10% MeOH:DCM (3×60 mL). The combined organics were washed sequentially with saturated aqueous NaHCO₃ solution and with H₂O (2×), then were dried over MgSO₄. Concentration provided a yellow solid that was triturated with 10% EtOAc:heptane to afford 5,6-dichloro-3-iodo-1H-pyrazolo[4,3-b]pyridine as a yellow solid (1.25 g). MS (M−H)⁻ 312.0.

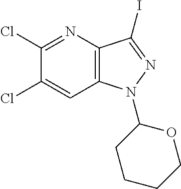 [SMILES: Clc1cc2c(nc1Cl)c(I)nn2C1CCCCO1]

### Preparation 14. 5,6-Dichloro-3-iodo-1-(tetrahydro-2H-pyran-2-yl)-1H-pyrazolo[4,3-b]pyridine

3,4-Dihydro-2H-pyran (0.82 mL, 9.0 mmol) and p-toluenesulfonic acid monohydrate (103 mg, 0.60 mmol) were added sequentially to a solution of 5,6-dichloro-3-iodo-1H-pyrazolo[4,3-b]pyridine (991 mg, 3.0 mmol) in THF (15 mL). The resulting mixture was heated at 50° C. for 9 h, then at ambient temperature for 12 h. The mixture was partitioned between saturated aqueous NaHCO₃ solution and EtOAc. The organic layer was washed with brine, dried over MgSO₄, and concentrated to afford a solid which was triturated with heptane to afford 5,6-dichloro-3-iodo-1-(tetrahydro-2H-pyran-2-yl)-1H-pyrazolo[4,3-b]pyridine as a beige solid (1.1 g).

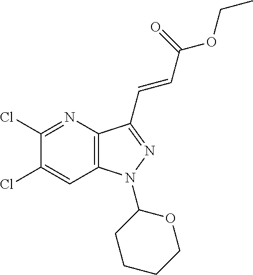 [SMILES: CCOC(=O)/C=C/c1nn(C2CCCCO2)c2cc(Cl)c(Cl)nc12]

### Preparation 15. Ethyl (E)-3-(5,6-dichloro-1-(tetrahydro-2H-pyran-2-yl)-1H-pyrazolo[4,3-b]pyridin-3-yl)acrylate

A solution of 5,6-dichloro-3-iodo-1-(tetrahydro-2H-pyran-2-yl)-1H-pyrazolo[4,3-b]pyridine (597 mg, 1.5 mmol), tri(o-tolyl)phosphine (120 mg, 0.37 mmol), and Pd(OAc)₂ (43 mg, 0.19 mmol) in DMF (7.5 mL) was sparged with N₂ for 5 min. Triethylamine (0.60 mL, 4.3 mmol) and ethyl acrylate (0.18 mL, 1.7 mmol) were added and the resulting mixture was heated at 80° C. for 4 h. An additional portion of ethyl acrylate (0.18 mL, 1.7 mmol) was added and heating at 80° C. was continued for an additional 19 h. The mixture was cooled and was combined with an analogous mixture from a reaction that was run on a scale of 80 mg (0.20 mmol) 5,6-dichloro-3-iodo-1-(tetrahydro-2H-pyran-2-yl)-1H-pyrazolo[4,3-b]pyridine. The combined materials were concentrated to remove DMF, and the resulting residue was partitioned between EtOAc and H₂O. The organic layer was washed sequentially with saturated aqueous NaHCO₃ solution, H₂O, and brine, then was concentrated. Silica gel chromatography (0-100% EtOAc:heptane) afforded ethyl (E)-3-(5,6-dichloro-1-(tetrahydro-2H-pyran-2-yl)-1H-pyrazolo[4,3-b]pyridin-3-yl)acrylate (483 mg). MS (M+H)⁺ 370.2.

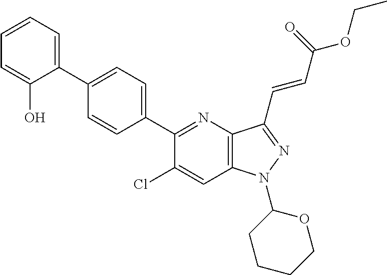 [SMILES: CCOC(=O)/C=C/c1nn(C2CCCCO2)c2cc(Cl)c(-c3ccc(-c4ccccc4O)cc3)nc12]

### Preparation 16. Ethyl (E)-3-(6-chloro-5-(2′-hydroxy-[1,1′-biphenyl]-4-yl)-1-(tetrahydro-2H-pyran-2-yl)-1H-pyrazolo[4,3-b]pyridin-3-yl)acrylate

A mixture of 4′-(4,4,5,5-tetramethyl-1,3,2-dioxaborolan-2-yl)-[1,1′-biphenyl]-2-ol (59 mg, 0.20 mmol), ethyl (E)-3-(5,6-dichloro-1-(tetrahydro-2H-pyran-2-yl)-1H-pyrazolo[4,3-b]pyridin-3-yl)acrylate (74 mg, 0.20 mmol), saturated aqueous NaHCO₃ solution (0.5 mL), and 1,2-dimethoxyethane (1 mL) was sparged with N₂ for 5 min. Pd(dppf)Cl₂ (8.2 mg, 0.010 mmol) was added and the mixture was heated at 60° C. for 22 h. The mixture was cooled and was combined with an analogous mixture from a reaction that was run on identical scale. The combined mixtures were partitioned between EtOAc and H₂O. The organic layer was washed with saturated aqueous NaHCO₃ solution, then was concentrated. Silica gel chromatography (0-100% EtOAc:heptane) afforded ethyl (E)-3-(6-chloro-5-(2′-hydroxy-[1,1′-biphenyl]-4-yl)-1-(tetrahydro-2H-pyran-2-yl)-1H-pyrazolo[4,3-b]pyridin-3-yl)acrylate (102 mg). MS (M+H)⁺504.3.

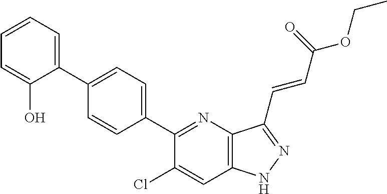 [SMILES: CCOC(=O)/C=C/c1n[nH]c2cc(Cl)c(-c3ccc(-c4ccccc4O)cc3)nc12]

### Preparation 17. Ethyl (E)-3-(6-chloro-5-(2′-hydroxy-[1,1′-biphenyl]-4-yl)-1H-pyrazolo[4,3-b]pyridin-3-yl)acrylate

TFA (0.30 mL, 4.0 mmol) was added to a solution of ethyl (E)-3-(6-chloro-5-(2′-hydroxy-[1,1′-biphenyl]-4-yl)-1-(tetrahydro-2H-pyran-2-yl)-1H-pyrazolo[4,3-b]pyridin-3-yl)acrylate (99 mg, 0.20 mmol) in DCM (2 mL). After 2.5 h, an additional portion of TFA (0.30 mL, 4.0 mmol) was added. After an additional 2.5 days, the mixture was concentrated and the resulting residue was partitioned between EtOAc and H₂O. The organic was washed sequentially with saturated aqueous NaHCO₃ solution and with brine, then was concentrated. Silica gel chromatography (0-100% EtOAc:heptane) afforded ethyl (E)-3-(6-chloro-5-(2′-hydroxy-[1,1′-biphenyl]-4-yl)-1H-pyrazolo[4,3-b]pyridin-3-yl)acrylate (58 mg).

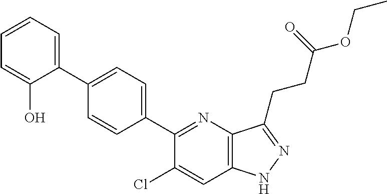 [SMILES: CCOC(=O)CCc1n[nH]c2cc(Cl)c(-c3ccc(-c4ccccc4O)cc3)nc12]

### Preparation 18. Ethyl 3-(6-chloro-5-(2′-hydroxy-[1,1′-biphenyl]-4-yl)-1H-pyrazolo[4,3-b]pyridin-3-yl)propanoate

Ethyl 3-(6-chloro-5-(2′-hydroxy-[1,1′-biphenyl]-4-yl)-1H-pyrazolo[4,3-b]pyridin-3-yl)propanoate was prepared from ethyl (E)-3-(6-chloro-5-(2′-hydroxy-[1,1′-biphenyl]-4-yl)-1H-pyrazolo[4,3-b]pyridin-3-yl)acrylate by a procedure analogous to Preparation 10. Ethyl 3-(6-chloro-5-(2′-hydroxy-[1,1′-biphenyl]-4-yl)-1H-pyrazolo[4,3-b]pyridin-3-yl)propanoate was purified by silica gel chromatography (0-100% EtOAc:heptane). MS (M+H)⁺ 422.4.

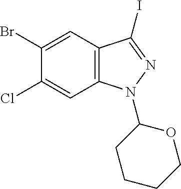 [SMILES: Clc1cc2c(cc1Br)c(I)nn2C1CCCCO1]

### Preparation 19. 5-Bromo-6-chloro-3-iodo-1-(tetrahydro-2H-pyran-2-yl)-1H-indazole

3,4-Dihydro-2H-pyran (7.5 mL, 82 mmol) and p-toluenesulfonic acid monohydrate (520 mg, 2.7 mmol) were added sequentially to a solution of 5-bromo-6-chloro-3-iodo-1H-indazole (9.76 g, 27.3 mmol) in DCM (50 mL). The resulting mixture was stirred for 16 h, then was concentrated. The resulting residue was treated with aqueous NaOH solution (1 M) to dissolve p-toluenesulfonic acid, and the remaining solids were collected by filtration to afford 5-bromo-6-chloro-3-iodo-1-(tetrahydro-2H-pyran-2-yl)-1H-indazole (3.58 g).

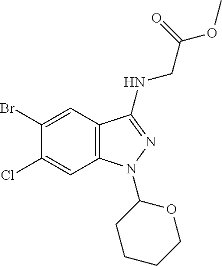 [SMILES: COC(=O)CNc1nn(C2CCCCO2)c2cc(Cl)c(Br)cc12]

### Preparation 20. Methyl (5-bromo-6-chloro-1-(tetrahydro-2H-pyran-2-yl)-1H-indazol-3-yl)glycinate

A mixture of 5-bromo-6-chloro-3-iodo-1-(tetrahydro-2H-pyran-2-yl)-1H-indazole (200 mg, 0.45 mmol), glycine methyl ester hydrochloride (63 mg, 0.50 mmol), CuI (8.6 mg, 0.045 mmol), N-(2,6-difluorophenyl)-6-hydroxypicolinamide (34 mg, 0.14 mmol), K₂CO₃ (250 mg, 1.8 mmol), and sodium ascorbate (9.0 mg, 0.045 mmol) in a sealed microwave vial was purged with N₂ (3×), then dioxane (2.27 mL) was added and the resulting mixture was heated at 100° C. for 18 h. The resulting mixture was cooled, diluted with EtOAc, and filtered through Celite. The filtrate was concentrated and the resulting residue was purified by silica gel chromatography (0-100% EtOAc:heptane) to afford methyl (5-bromo-6-chloro-1-(tetrahydro-2H-pyran-2-yl)-1H-indazol-3-yl)glycinate as a solid (115 mg). MS (M+H)⁺ 404.1.

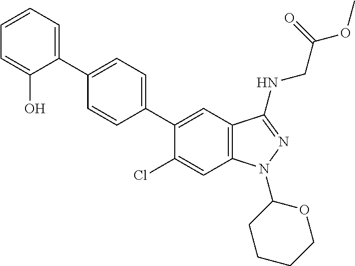 [SMILES: COC(=O)CNc1nn(C2CCCCO2)c2cc(Cl)c(-c3ccc(-c4ccccc4O)cc3)cc12]

### Preparation 21. Methyl (6-chloro-5-(2′-hydroxy-[1,1′-biphenyl]-4-yl)-1-(tetrahydro-2H-pyran-2-yl)-1H-indazol-3-yl)glycinate

A mixture of 4′-(4,4,5,5-tetramethyl-1,3,2-dioxaborolan-2-yl)-[1,1′-biphenyl]-2-ol (110 mg, 0.37 mmol), K₂CO₃ (118 mg, 0.86 mmol), and Pd(dppf)Cl₂—CH₂Cl₂ (23 mg, 0.029 mmol) in a sealed microwave vial was purged with N₂ (3×). A solution of methyl (5-bromo-6-chloro-1-(tetrahydro-2H-pyran-2-yl)-1H-indazol-3-yl)glycinate (115 mg, 0.029 mmol) in dioxane (2.0 mL) and then H₂O (0.5 mL) were sequentially added and the resulting mixture was heated at 90° C. for 1.75 h. The mixture was cooled and was filtered through Celite, rinsing with EtOAc, and the filtrate was concentrated to remove organic solvents. The resulting mixture was extracted with DCM (3×). The combined organics were dried over MgSO₄ and were concentrated. Silica gel chromatography (0-100% EtOAc:heptane) afforded methyl (6-chloro-5-(2′-hydroxy-[1,1′-biphenyl]-4-yl)-1-(tetrahydro-2H-pyran-2-yl)-1H-indazol-3-yl)glycinate (83 mg). MS (M+H)⁺ 492.3.

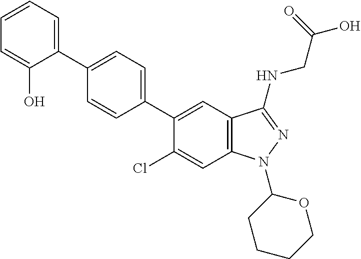 [SMILES: O=C(O)CNc1nn(C2CCCCO2)c2cc(Cl)c(-c3ccc(-c4ccccc4O)cc3)cc12]

### Preparation 22. (6-Chloro-5-(2′-hydroxy-[1,1′-biphenyl]-4-yl)-1-(tetrahydro-2H-pyran-2-yl)-1H-indazol-3-yl)glycine

Aqueous NaOH solution (1 M, 0.50 mL, 0.50 mmol) was added to a mixture of methyl (6-chloro-5-(2′-hydroxy-[1,1′-biphenyl]-4-yl)-1-(tetrahydro-2H-pyran-2-yl)-1H-indazol-3-yl)glycinate (20 mg, 0.041 mmol) and MeOH (0.5 mL). The mixture was heated at 50° C. for 1 h, then was cooled and was diluted with aqueous HCl solution (1 M) until a precipitate formed.

The mixture was extracted with 20% MeOH:DCM (2 mL), and the organic layer was concentrated to afford (6-chloro-5-(2′-hydroxy-[1,1′-biphenyl]-4-yl)-1-(tetrahydro-2H-pyran-2-yl)-1H-indazol-3-yl)glycine (22 mg) which was used without further purification. MS (M+H)⁺ 478.2.

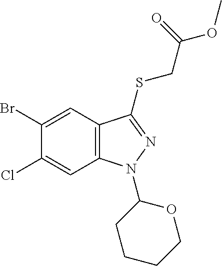 [SMILES: COC(=O)CSc1nn(C2CCCCO2)c2cc(Cl)c(Br)cc12]

### Preparation 23. Methyl 2-((5-bromo-6-chloro-1-(tetrahydro-2H-pyran-2-yl)-1H-indazol-3-yl)thio)acetate

To a sealed microwave vial containing Pd₂(dba)₃ (41 mg, 0.044 mmol) and Xantphos (51 mg, 0.088 mmol) under an atmosphere of N₂ were added sequentially a solution of 5-bromo-6-chloro-3-iodo-1-(tetrahydro-2H-pyran-2-yl)-1H-indazole (196 mg, 0.44 mmol) in toluene (2 mL), N,N-diisopropylethylamine (78 μL, 0.44 mmol), and methyl 2-mercaptoacetate (40 μL, 0.44 mmol). The resulting mixture was heated at 45° C. for 1.5 h, then was cooled and was diluted with H₂O. The organic layer was collected, and the aqueous layer was extracted with EtOAc (2×). The combined organics were washed with brine, dried over MgSO₄, and concentrated. Silica gel chromatography (0-100% EtOAc:heptane) afforded methyl 2-((5-bromo-6-chloro-1-(tetrahydro-2H-pyran-2-yl)-1H-indazol-3-yl)thio)acetate as a solid (114 mg). MS (M+H)⁺ 421.2.

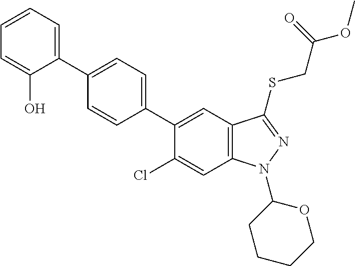 [SMILES: COC(=O)CSc1nn(C2CCCCO2)c2cc(Cl)c(-c3ccc(-c4ccccc4O)cc3)cc12]

### Preparation 24. Methyl 2-((6-chloro-5-(2′-hydroxy-[1,1′-biphenyl]-4-yl)-1-(tetrahydro-2H-pyran-2-yl)-1H-indazol-3-yl)thio)acetate

Methyl 2-((6-chloro-5-(2′-hydroxy-[1,1′-biphenyl]-4-yl)-1-(tetrahydro-2H-pyran-2-yl)-1H-indazol-3-yl)thio)acetate was prepared from methyl 2-((5-bromo-6-chloro-1-(tetrahydro-2H-pyran-2-yl)-1H-indazol-3-yl)thio)acetate by an analogous method to Preparation 21. MS (M+H)⁺ 509.4.

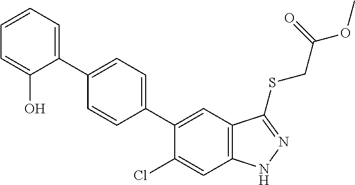 [SMILES: COC(=O)CSc1n[nH]c2cc(Cl)c(-c3ccc(-c4ccccc4O)cc3)cc12]

### Preparation 25. Methyl 2-((6-chloro-5-(2′-hydroxy-[1,1′-biphenyl]-4-yl)-1H-indazol-3-yl)thio)acetate

TFA (0.30 mL, 3.9 mmol) was added to a mixture of methyl 2-((6-chloro-5-(2′-hydroxy-[1,1′-biphenyl]-4-yl)-1-(tetrahydro-2H-pyran-2-yl)-1H-indazol-3-yl)thio)acetate (16 mg, 0.031 mmol) and DCM (0.5 mL). After 2.5 h, the mixture was concentrated to afford methyl 2-((6-chloro-5-(2′-hydroxy-[1,1′-biphenyl]-4-yl)-1H-indazol-3-yl)thio)acetate which was used without further purification. MS (M+H)⁺ 425.3.

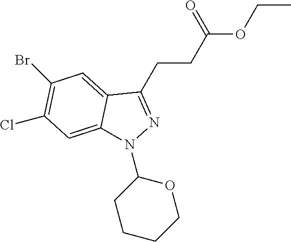 [SMILES: CCOC(=O)CCc1nn(C2CCCCO2)c2cc(Cl)c(Br)cc12]

### Preparation 26. Ethyl 3-(5-bromo-6-chloro-1-(tetrahydro-2H-pyran-2-yl)-1H-indazol-3-yl)propanoate

3,4-Dihydro-2H-pyran (8.77 mL, 96.5 mmol) and p-toluenesulfonic acid monohydrate (1.66 g, 9.65 mmol) were added sequentially to a solution of ethyl 3-(5-bromo-6-chloro-1H-indazol-3-yl)propanoate (16.0 g, 48.3 mmol) in THF (100 mL). The resulting mixture was heated at 50° C. for 4 h, then was cooled. The mixture was partitioned between saturated aqueous NaHCO₃ solution (30 mL) and EtOAc (3×100 mL). The combined organics were dried over Na₂SO₄ and concentrated. Silica gel chromatography (0-80% EtOAc:petroleum ether) afforded ethyl 3-(5-bromo-6-chloro-1-(tetrahydro-2H-pyran-2-yl)-1H-indazol-3-yl)propanoate as a solid (16.5 g). MS (M+H)+417.1.

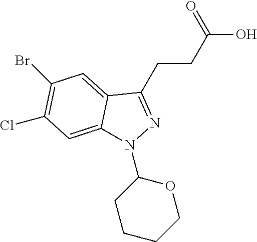 [SMILES: O=C(O)CCc1nn(C2CCCCO2)c2cc(Cl)c(Br)cc12]

### Preparation 27. 3-(5-Bromo-6-chloro-1-(tetrahydro-2H-pyran-2-yl)-1H-indazol-3-yl)propanoic acid

LiOH—H₂O (303 mg, 7.2 mmol) was added to a solution of ethyl 3-(5-bromo-6-chloro-1-(tetrahydro-2H-pyran-2-yl)-1H-indazol-3-yl)propanoate (1.0 g, 2.4 mmol) in THF (10 mL), MeOH (10 mL), and H₂O (10 mL) at 15° C. After 3 h, the mixture was concentrated to remove organic solvents, then the pH was adjusted to ˜3 by addition of aqueous HCl solution. The resulting white solids were collected and were dried to afford 3-(5-bromo-6-chloro-1-(tetrahydro-2H-pyran-2-yl)-1H-indazol-3-yl)propanoic acid (0.90 g). MS (M+H)⁺ 388.9.

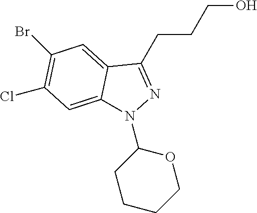 [SMILES: OCCCc1nn(C2CCCCO2)c2cc(Cl)c(Br)cc12]

### Preparation 28. 3-(5-Bromo-6-chloro-1-(tetrahydro-2H-pyran-2-yl)-1H-indazol-3-yl)propan-1-ol

Borane tetrahydrofuran complex (1 M solution in THF, 6.96 mL, 6.96 mmol) was added dropwise to a solution of 3-(5-bromo-6-chloro-1-(tetrahydro-2H-pyran-2-yl)-1H-indazol-3-yl)propanoic acid (0.90 g, 2.32 mmol) in THF (20 mL) at 0° C. The resulting mixture was stirred for 16 h at 15° C. MeOH was added and the mixture was concentrated. The resulting residue was partitioned between H₂O (30 mL) and EtOAc (100 mL, then 60 mL). The combined organics were washed with brine (2×30 mL) and were concentrated to afford 3-(5-bromo-6-chloro-1-(tetrahydro-2H-pyran-2-yl)-1H-indazol-3-yl)propan-1-ol (0.80 g) as a solid which was used without further purification. MS (M+H)⁺ 375.0.

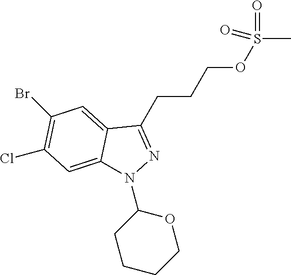 [SMILES: CS(=O)(=O)OCCCc1nn(C2CCCCO2)c2cc(Cl)c(Br)cc12]

### Preparation 29. 3-(5-Bromo-6-chloro-1-(tetrahydro-2H-pyran-2-yl)-1H-indazol-3-yl)propyl methanesulfonate

Triethylamine (1.49 mL, 10.7 mmol) and methanesulfonic anhydride (1.49 g, 8.56 mmol) were added sequentially to a solution of 3-(5-bromo-6-chloro-1-(tetrahydro-2H-pyran-2-yl)-1H-indazol-3-yl)propan-1-ol (0.80 g, 2.1 mmol) in DCM (45 mL) at 0° C. The resulting mixture was stirred for 18 h at 10° C., then was partitioned between DCM (30 mL) and H₂O (2×20 mL). The combined organics were washed sequentially with aqueous citric acid solution (0.5 M, 20 mL) and with brine (20 mL), then were dried over Na₂SO₄ and were concentrated to afford 3-(5-bromo-6-chloro-1-(tetrahydro-2H-pyran-2-yl)-1H-indazol-3-yl)propyl methanesulfonate (0.96 g) as an oil that was used without further purification. MS (M+H)⁺ 452.9.

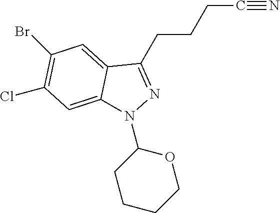 [SMILES: N#CCCCc1nn(C2CCCCO2)c2cc(Cl)c(Br)cc12]

### Preparation 30. 4-(5-Bromo-6-chloro-1-(tetrahydro-2H-pyran-2-yl)-1H-indazol-3-yl)butanenitrile

Trimethylsilyl cyanide (425 mg, 4.28 mmol) and KF (249 mg, 4.28 mmol) were added sequentially to a solution of 3-(5-bromo-6-chloro-1-(tetrahydro-2H-pyran-2-yl)-1H-indazol-3-yl)propyl methanesulfonate (0.96 g, 2.1 mmol) in DMF (20 mL). The resulting mixture was heated for 16 h at 90° C., then was cooled and was partitioned between EtOAc and H₂O. The organic layer was washed with brine, then was dried over Na₂SO₄ and was concentrated. Silica gel chromatography (15% EtOAc:petroleum ether) afforded 4-(5-bromo-6-chloro-1-(tetrahydro-2H-pyran-2-yl)-1H-indazol-3-yl)butanenitrile (0.30 g) as an oil. MS (M+H)+381.9.

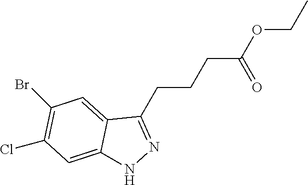 [SMILES: CCOC(=O)CCCc1n[nH]c2cc(Cl)c(Br)cc12]

### Preparation 31. Ethyl 4-(5-bromo-6-chloro-1H-indazol-3-yl)butanoate

Sulfuric acid (1 mL) was added dropwise to a solution of 4-(5-bromo-6-chloro-1-(tetrahydro-2H-pyran-2-yl)-1H-indazol-3-yl)butanenitrile (0.30 g, 0.78 mmol) in EtOH (5 mL), and the resulting mixture was heated at 100° C. for 16 h, then was cooled and was concentrated. Saturated aqueous NaHCO₃ solution (30 mL) was added, and the mixture was extracted with EtOAc (3×100 mL). The combined organics were washed with brine, dried over Na₂SO₄, and concentrated. Silica gel chromatography (0-50% EtOAc:petroleum ether) afforded ethyl 4-(5-bromo-6-chloro-1H-indazol-3-yl)butanoate (150 mg) as an oil. MS (M+H)⁺ 346.9.

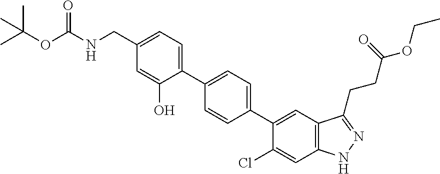 [SMILES: CCOC(=O)CCc1n[nH]c2cc(Cl)c(-c3ccc(-c4ccc(CNC(=O)OC(C)(C)C)cc4O)cc3)cc12]

### Preparation 32. Ethyl 3-(5-(4′-(((tert-butoxycarbonyl)amino)methyl)-2′-hydroxy-[1,1′-biphenyl]-4-yl)-6-chloro-1H-indazol-3-yl)propanoate

Ethyl 3-(5-(4′-(((tert-butoxycarbonyl)amino)methyl)-2′-hydroxy-[1,1′-biphenyl]-4-yl)-6-chloro-1H-indazol-3-yl)propanoate was prepared from tert-butyl (4-bromo-3-hydroxybenzyl)carbamate by a procedure analogous to Preparation 7. MS (M+H)⁺ 550.1.

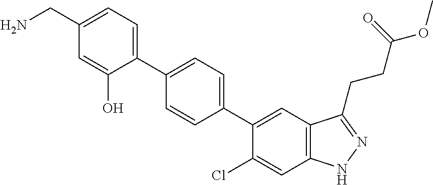 [SMILES: COC(=O)CCc1n[nH]c2cc(Cl)c(-c3ccc(-c4ccc(CN)cc4O)cc3)cc12]

### Preparation 33. Methyl 3-(5-(4′-(aminomethyl)-2′-hydroxy-[1,1′-biphenyl]-4-yl)-6-chloro-1H-indazol-3-yl)propanoate

A solution of HCl in dioxane (4 M, 2.0 mL, 8.0 mmol) was added to a solution of ethyl 3-(5-(4′-(((tert-butoxycarbonyl)amino)methyl)-2′-hydroxy-[1,1′-biphenyl]-4-yl)-6-chloro-1H-indazol-3-yl)propanoate (55 mg, 0.10 mmol) in MeOH (4 mL). After 3 h at 20° C., the resulting mixture was concentrated to afford methyl 3-(5-(4′-(aminomethyl)-2′-hydroxy-[1,1′-biphenyl]-4-yl)-6-chloro-1H-indazol-3-yl)propanoate (44 mg) which was used without further purification. MS (M+H)⁺ 437.3.

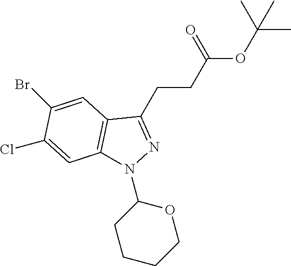 [SMILES: CC(C)(C)OC(=O)CCc1nn(C2CCCCO2)c2cc(Cl)c(Br)cc12]

### Preparation 34. tert-Butyl 3-(5-bromo-6-chloro-1-(tetrahydro-2H-pyran-2-yl)-1H-indazol-3-yl)propanoate

tert-Butyl 3-(5-bromo-6-chloro-1-(tetrahydro-2H-pyran-2-yl)-1H-indazol-3-yl)propanoate was prepared from 5-bromo-6-chloro-1H-indazole-3-carbaldehyde and (tert-butoxycarbonylmethylene)triphenylphosphorane by procedures analogous to Preparations 9, 10, and 26. MS (M+H)⁺445.1.

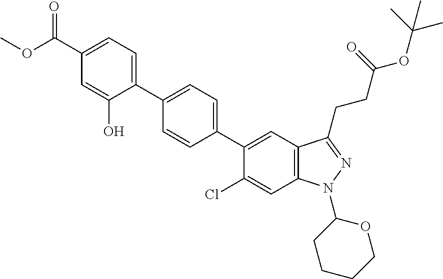 [SMILES: COC(=O)c1ccc(-c2ccc(-c3cc4c(CCC(=O)OC(C)(C)C)nn(C5CCCCO5)c4cc3Cl)cc2)c(O)c1]

### Preparation 35. Methyl 4′-(3-(3-(tert-butoxy)-3-oxopropyl)-6-chloro-1-(tetrahydro-2H-pyran-2-yl)-1H-indazol-5-yl)-2-hydroxy-[1,1′-biphenyl]-4-carboxylate

Methyl 4′-(3-(3-(tert-butoxy)-3-oxopropyl)-6-chloro-1-(tetrahydro-2H-pyran-2-yl)-1H-indazol-5-yl)-2-hydroxy-[1,1′-biphenyl]-4-carboxylate was prepared from tert-butyl 3-(5-bromo-6-chloro-1-(tetrahydro-2H-pyran-2-yl)-1H-indazol-3-yl)propanoate, 1,4-bis(4,4,5,5-tetramethyl-1,3,2-dioxaborolan-2-yl)benzene, and methyl 4-bromo-3-hydroxybenzoate by procedures analogous to Preparations 3 and 7. MS (M+H)⁺ 591.5.

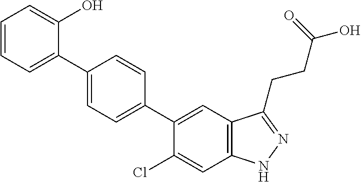 [SMILES: O=C(O)CCc1n[nH]c2cc(Cl)c(-c3ccc(-c4ccccc4O)cc3)cc12]

### Example 1. 3-(6-Chloro-5-(2′-hydroxy-[1,1′-biphenyl]-4-yl)-1H-indazol-3-yl)propanoic acid

Aqueous NaOH solution (1.0 M, 26.0 mL, 26.0 mmol) was added to a solution of ethyl 3-(6-chloro-5-(2′-hydroxy-[1,1′-biphenyl]-4-yl)-1H-indazol-3-yl)propanoate (2.14 g, 5.08 mmol) in inhibitor-free THF (12.5 mL) and EtOH (12.5 mL). After 4 h, the mixture was concentrated to remove the organics, then the aqueous residue was diluted with H₂O (25 mL). The resulting solution was heated to 100° C. and was held at that temperature for 15 min. An aqueous HCl solution (1.0 M, 27 mL, 27 mmol) was added dropwise to the heated solution, resulting in formation of a precipitate. The mixture was heated at reflux for 16 h, then was cooled to ambient temperature and was stirred for an additional 4 h. The solids were collected by filtration and were dried under a flow of N₂ gas to afford 3-(6-chloro-5-(2′-hydroxy-[1,1′-biphenyl]-4-yl)-1H-indazol-3-yl)propanoic acid as a crystalline white solid (1.6 g).

¹H NMR (400 MHz, DMSO-d₆) δ 12.84 (s, 1H), 12.11 (s, 1H), 9.58 (s, 1H), 7.83 (s, 1H), 7.68 (s, 1H), 7.63 (d, 2H), 7.48 (d, 2H), 7.33 (dd, 1H), 7.18 (m, 1H), 6.97 (d, 1H), 6.91 (t, 1H), 3.16 (t, 2H), 2.72 (t, 2H); MS (M+H)⁺393.1.

The following examples were synthesized by analogous methods and starting materials as described for 3-(6-chloro-5-(2′-hydroxy-[1,1′-biphenyl]-4-yl)-1H-indazol-3-yl)propanoic acid.

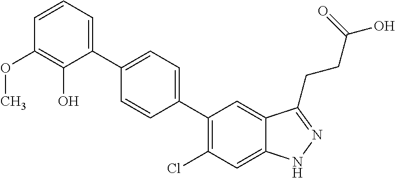 [SMILES: COc1cccc(-c2ccc(-c3cc4c(CCC(=O)O)n[nH]c4cc3Cl)cc2)c1O]

### Example 2. 3-(6-Chloro-5-(2′-hydroxy-3′-methoxy-[1,1′-biphenyl]-4-yl)-1H-indazol-3-yl)propanoic acid

Synthesized from 1,4-dibromobenzene, (2-hydroxy-3-methoxyphenyl)boronic acid, and 1,4-bis(5,5-dimethyl-1,3,2-dioxaborinan-2-yl)benzene by analogous methods as described for 3-(6-chloro-5-(2′-hydroxy-[1,1′-biphenyl]-4-yl)-1H-indazol-3-yl)propanoic acid.

¹H NMR (400 MHz, DMSO-d₆) δ 12.84 (s, 1H), 12.12 (s, 1H), 8.67 (s, 1H), 7.83 (s, 1H), 7.68 (s, 1H), 7.62 (d, 2H), 7.48 (d, 2H), 6.97 (m, 2H), 6.88 (m, 1H), 3.86 (s, 3H), 3.16 (t, 2H), 2.72 (t, 2H); MS (M+H)⁺ 423.3.

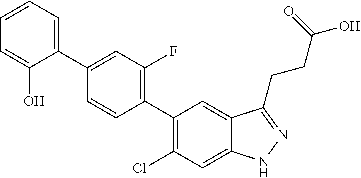 [SMILES: O=C(O)CCc1n[nH]c2cc(Cl)c(-c3ccc(-c4ccccc4O)cc3F)cc12]

### Example 3. 3-(6-Chloro-5-(3-fluoro-2′-hydroxy-[1,1′-biphenyl]-4-yl)-1H-indazol-3-yl)propanoic acid

Synthesized from 1-bromo-2-fluoro-4-iodobenzene by analogous methods as described for 3-(6-chloro-5-(2′-hydroxy-[1,1′-biphenyl]-4-yl)-1H-indazol-3-yl)propanoic acid.

¹H NMR (400 MHz, MeOD) δ 7.76 (s, 1H), 7.62 (s, 1H), 7.43 (m, 2H), 7.36 (m, 2H), 7.17 (m, 1H), 6.91 (m, 2H), 3.25 (t, 2H), 2.79 (t, 2H); MS (M+H)⁺ 411.2.

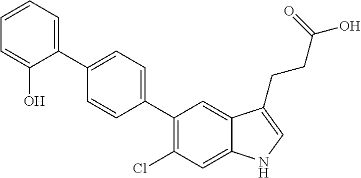 [SMILES: O=C(O)CCc1c[nH]c2cc(Cl)c(-c3ccc(-c4ccccc4O)cc3)cc12]

### Example 4. 3-(6-Chloro-5-(2′-hydroxy-[1,1′-biphenyl]-4-yl)-1H-indol-3-yl)propanoic acid

Synthesized from 5-bromo-6-chloro-1H-indole-3-carbaldehyde by analogous methods as described for 3-(6-chloro-5-(2′-hydroxy-[1,1′-biphenyl]-4-yl)-1H-indazol-3-yl)propanoic acid. ¹H NMR (400 MHz, DMSO-d₆) δ 10.99 (s, 1H), 9.63 (s, 1H), 8.32 (s, 1H), 7.56 (m, 6H), 7.32 (m, 1H), 7.20 (m, 2H), 6.93 (m, 2H), 2.93 (t, 2H), 2.56 (t, 2H); MS (M+H)⁺392.0.

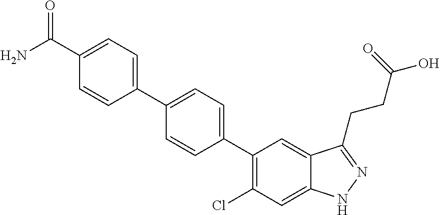 [SMILES: NC(=O)c1ccc(-c2ccc(-c3cc4c(CCC(=O)O)n[nH]c4cc3Cl)cc2)cc1]

### Example 5. 3-(5-(4′-Carbamoyl-[1,1′-biphenyl]-4-yl)-6-chloro-1H-indazol-3-yl)propanoic acid

LiOH—H₂O (27 mg, 0.64 mmol) was added to a mixture of ethyl 3-(5-(4′-carbamoyl-2′-hydroxy-[1,1′-biphenyl]-4-yl)-6-chloro-1H-indazol-3-yl)propanoate (95 mg, 0.21 mmol) in MeOH (3 mL) and H₂O (1 mL), and the resulting mixture was stirred for 16 h. The mixture was concentrated to remove the organics, then the aqueous residue was acidified to pH ˜3 by addition of aqueous HCl solution (1 M). The resulting solid was collected, rinsed with H₂O, and dried to afford 3-(5-(4′-carbamoyl-[1,1′-biphenyl]-4-yl)-6-chloro-1H-indazol-3-yl)propanoic acid as a solid (72 mg).

¹H NMR (400 MHz, MeOD) δ 7.99 (d, 2H), 7.85 (s, 1H), 7.82 (d, 2H), 7.77 (d, 2H), 7.64 (s, 1H), 7.58 (d, 2H), 3.27 (m, 2H), 2.70 (m, 2H); MS (M+H)⁺ 420.1.

The following examples were synthesized by analogous methods and starting materials as described for 3-(5-(4′-carbamoyl-[1,1′-biphenyl]-4-yl)-6-chloro-1H-indazol-3-yl)propanoic acid.

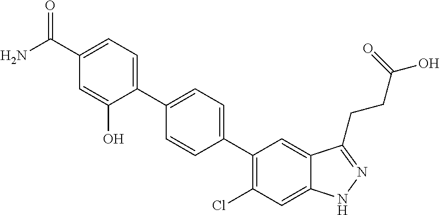 [SMILES: NC(=O)c1ccc(-c2ccc(-c3cc4c(CCC(=O)O)n[nH]c4cc3Cl)cc2)c(O)c1]

### Example 6. 3-(5-(4′-Carbamoyl-2′-hydroxy-[1,1′-biphenyl]-4-yl)-6-chloro-1H-indazol-3-yl)propanoic acid

Synthesized from 4-bromo-3-hydroxybenzamide by analogous methods as described for 3-(5-(4′-carbamoyl-[1,1′-biphenyl]-4-yl)-6-chloro-1H-indazol-3-yl)propanoic acid.

¹H NMR (400 MHz, MeOD) δ 7.83 (s, 1H), 7.68 (m, 2H), 7.63 (s, 1H), 7.50 (m, 2H), 7.43 (m, 3H), 3.27 (m, 2H), 2.71 (m, 2H); MS (M+H)⁺ 436.1.

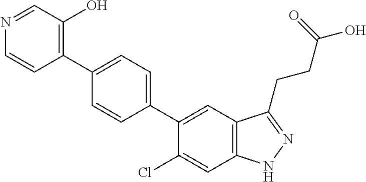 [SMILES: O=C(O)CCc1n[nH]c2cc(Cl)c(-c3ccc(-c4ccncc4O)cc3)cc12]

### Example 7. 3-(6-Chloro-5-(4-(3-hydroxypyridin-4-yl)phenyl)-1H-indazol-3-yl)propanoic acid

Synthesized from 4-bromopyridin-3-ol by analogous methods as described for 3-(5-(4′-carbamoyl-[1,1′-biphenyl]-4-yl)-6-chloro-1H-indazol-3-yl)propanoic acid.

¹H NMR (400 MHz, MeOD) δ 8.19 (s, 1H), 8.10 (d, 1H), 7.82 (s, 1H), 7.78 (d, 2H), 7.65 (s, 1H), 7.56 (d, 2H), 7.46 (d, 1H), 3.27 (m, 2H), 2.78 (m, 2H); MS (M+H)⁺ 394.2.

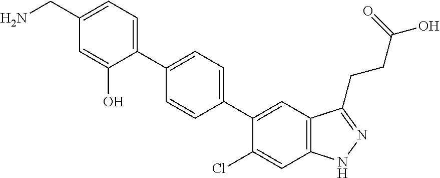 [SMILES: NCc1ccc(-c2ccc(-c3cc4c(CCC(=O)O)n[nH]c4cc3Cl)cc2)c(O)c1]

### Example 8. 3-(5-(4′-(Aminomethyl)-2′-hydroxy-[1,1′-biphenyl]-4-yl)-6-chloro-1H-indazol-3-yl)propanoic acid

NaOH (20 mg, 0.51 mmol) was added to methyl 3-(5-(4′-(aminomethyl)-2′-hydroxy-[1,1′-biphenyl]-4-yl)-6-chloro-1H-indazol-3-yl)propanoate (44 mg, 0.10 mmol), MeOH (4 mL), and H₂O (2 mL) at 20° C. After 3 h, the pH was adjusted to ˜4 with 2 M hydrochloric acid. The mixture was concentrated, and the residue was purified by reversed-phase HPLC to afford 3-(5-(4′-(aminomethyl)-2′-hydroxy-[1,1′-biphenyl]-4-yl)-6-chloro-1H-indazol-3-yl)propanoic acid. MS (M+H)⁺ 422.1. LC Method A, r.t. 0.60 min.

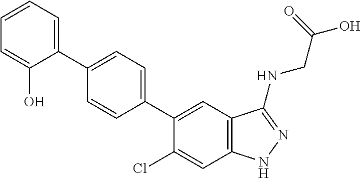 [SMILES: O=C(O)CNc1n[nH]c2cc(Cl)c(-c3ccc(-c4ccccc4O)cc3)cc12]

### Example 19. (6-Chloro-5-(2′-hydroxy-[1,1′-biphenyl]-4-yl)-1H-indazol-3-yl)glycine

TFA (0.20 mL, 2.6 mmol) was added to a mixture of (6-chloro-5-(2′-hydroxy-[1,1′-biphenyl]-4-yl)-1-(tetrahydro-2H-pyran-2-yl)-1H-indazol-3-yl)glycine (22 mg, 0.046 mmol) and DCM (1.0 mL). After 3.5 h, the mixture was concentrated and the resulting residue was purified by reversed-phase HPLC to afford (6-chloro-5-(2′-hydroxy-[1,1′-biphenyl]-4-yl)-1H-indazol-3-yl)glycine (3.4 mg). MS (M+H)⁺394.3. LC Method G, r.t. 2.60 min.

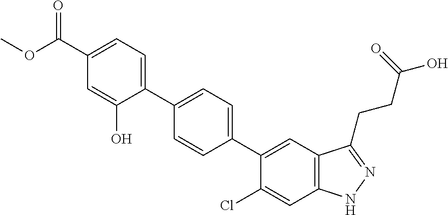 [SMILES: COC(=O)c1ccc(-c2ccc(-c3cc4c(CCC(=O)O)n[nH]c4cc3Cl)cc2)c(O)c1]

### Example 31. 3-(6-Chloro-5-(2′-hydroxy-4′-(methoxycarbonyl)-[1,1′-biphenyl]-4-yl)-1H-indazol-3-yl)propanoic acid

A mixture of methyl 4′-(3-(3-(tert-butoxy)-3-oxopropyl)-6-chloro-1-(tetrahydro-2H-pyran-2-yl)-1H-indazol-5-yl)-2-hydroxy-[1,1′-biphenyl]-4-carboxylate (40 mg, 0.068 mmol) and a solution of HCl in dioxane (4 M, 5.0 mL, 20 mmol) was stirred at 25° C. for 16 h. The mixture was concentrated and the resulting residue was purified by reversed-phase HPLC to afford 3-(6-chloro-5-(2′-hydroxy-4′-(methoxycarbonyl)-[1,1′-biphenyl]-4-yl)-1H-indazol-3-yl)propanoic acid (17 mg). ¹H NMR (400 MHz, MeOD) δ 7.82 (s, 1H), 7.69 (d, 2H), 7.65-7.55 (m, 3H), 7.51 (d, 2H), 7.46 (d, 1H), 3.91 (s, 3H), 3.26 (t, 2H), 2.76 (t, 2H); MS (M+H)⁺ 451.3.

The Examples in Table 1 were prepared using methods analogous to the methods described above.

TABLE 1
AMPK-activating compounds

| | | | | MS (M + H)⁺ |
|---|---|---|---|---|
| | | | Analogous | LC method |
| Ex# | Structure | IUPAC Name | Synthesis | r.t. |
| 9 | 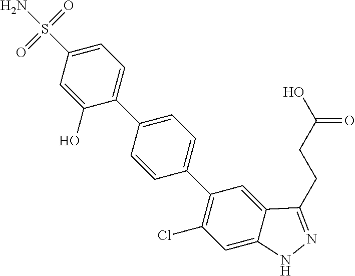 [SMILES: NS(=O)(=O)c1ccc(-c2ccc(-c3cc4c(CCC(=O)O)n[nH]c4cc3Cl)cc2)c(O)c1] | 3-(6-chloro-5-(2′- hydroxy-4′-sulfam- oyl-[1,1′-biphenyl]- 4-yl)-1H-indazol-3- yl)propanoic acid | P7 | 472.2 Method A 0.661 min |
| 10 | 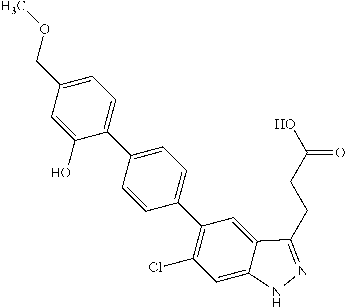 [SMILES: COCc1ccc(-c2ccc(-c3cc4c(CCC(=O)O)n[nH]c4cc3Cl)cc2)c(O)c1] | 3-(6-chloro-5-(2′- hydroxy-4′-(meth- oxymethyl)-[1,1′- biphenyl]-4-yl)- 1H-indazol-3-yl)- propanoic acid | P7 | 437.3 Method A 0.813 min |
| 11 | 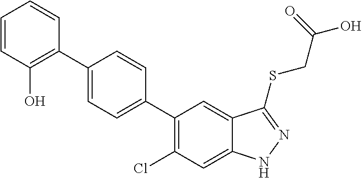 [SMILES: O=C(O)CSc1n[nH]c2cc(Cl)c(-c3ccc(-c4ccccc4O)cc3)cc12] | 2-((6-chloro-5-(2′- hydroxy-[1,1′- biphenyl]-4-yl)- 1H-indazol-3-yl)- thio)acetic acid | P25 | 411.1 Method G 2.92 min |
| 12 | 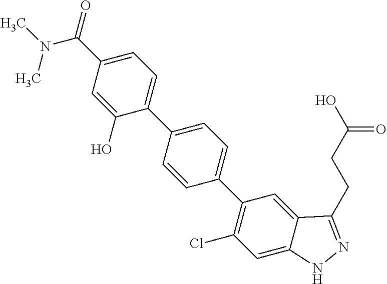 [SMILES: CN(C)C(=O)c1ccc(-c2ccc(-c3cc4c(CCC(=O)O)n[nH]c4cc3Cl)cc2)c(O)c1] | 3-(6-chloro-5-(4′- (dimethylcarbam- oyl)-2′-hydroxy- [1,1′-biphenyl]-4- yl)-1H-indazol-3- yl)propanoic acid | P7 | 464.3 Method A 0.702 min |
| 13 | 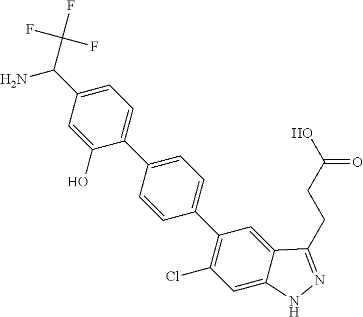 [SMILES: NC(c1ccc(-c2ccc(-c3cc4c(CCC(=O)O)n[nH]c4cc3Cl)cc2)c(O)c1)C(F)(F)F] | 3-(5-(4′-(1-amino- 2,2,2-trifluoroeth- yl)-2′-hydroxy-[1,1′- biphenyl]-4-yl)-6- chloro-1H-indazol- 3-yl)propanoic acid | P7 | 490.2 Method A 0.75 min |
| 14 | 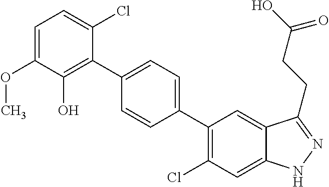 [SMILES: COc1ccc(Cl)c(-c2ccc(-c3cc4c(CCC(=O)O)n[nH]c4cc3Cl)cc2)c1O] | 3-(6-chloro-5-(6′- chloro-2′-hydroxy- 3′-methoxy-[1,1′- biphenyl]-4-yl)- 1H-indazol-3-yl)- propanoic acid | P7 | 457.2 Method A 0.829 min |
| 15 | 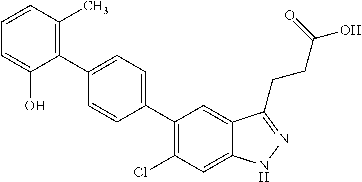 [SMILES: Cc1cccc(O)c1-c1ccc(-c2cc3c(CCC(=O)O)n[nH]c3cc2Cl)cc1] | 3-(6-chloro-5-(2′- hydroxy-6′-methyl- [1,1′-biphenyl]-4- yl)-1H-indazol-3- yl)propanoic acid | P7 | 407 Method C 2.938 min |
| 16 | 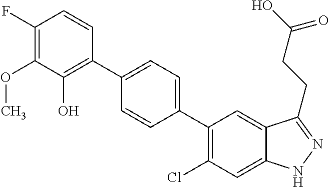 [SMILES: COc1c(F)ccc(-c2ccc(-c3cc4c(CCC(=O)O)n[nH]c4cc3Cl)cc2)c1O] | 3-(6-chloro-5-(4′- fluoro-2′-hydroxy- 3′-methoxy-[1,1′- biphenyl]-4-yl)- 1H-indazol-3-yl)- propanoic acid | P7 | 441.3 Method A 0.855 min |
| 17 | 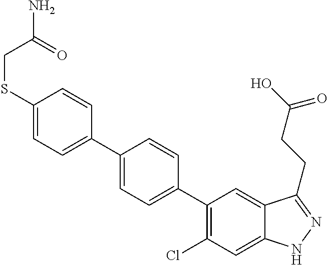 [SMILES: NC(=O)CSc1ccc(-c2ccc(-c3cc4c(CCC(=O)O)n[nH]c4cc3Cl)cc2)cc1] | 3-(5-(4′-((2-amino- 2-oxoethyl)thio)- [1,1′-biphenyl]-4- yl)-6-chloro-1H- indazol-3-yl)- propanoic acid | P7 | 466 Method C 2.563 min |
| 18 | 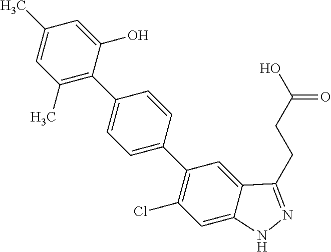 [SMILES: Cc1cc(C)c(-c2ccc(-c3cc4c(CCC(=O)O)n[nH]c4cc3Cl)cc2)c(O)c1] | 3-(6-chloro-5-(2′- hydroxy-4′,6′- dimethyl-[1,1′- biphenyl]-4-yl)- 1H-indazol-3-yl)- propanoic acid | P7 | 421 Method C 3.076 min |
| 20 | 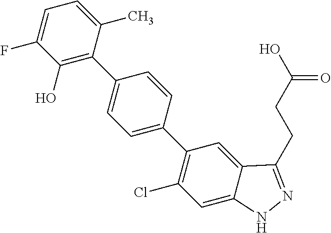 [SMILES: Cc1ccc(F)c(O)c1-c1ccc(-c2cc3c(CCC(=O)O)n[nH]c3cc2Cl)cc1] | 3-(6-chloro-5-(3′- fluoro-2′-hydroxy- 6′-methyl-[1,1′- biphenyl]-4-yl)- 1H-indazol-3-yl)- propanoic acid | P7 | 425 Method C 3 min |
| 21 | 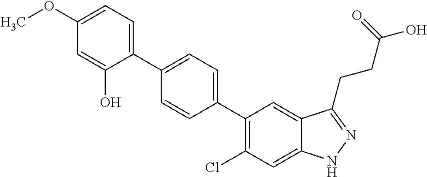 [SMILES: COc1ccc(-c2ccc(-c3cc4c(CCC(=O)O)n[nH]c4cc3Cl)cc2)c(O)c1] | 3-(6-chloro-5-(2′- hydroxy-4′-meth- oxy-[1,1′-biphenyl]- 4-yl)-1H-indazol-3- yl)propanoic acid | P7 | 423 Method C 2.795 min |
| 22 | 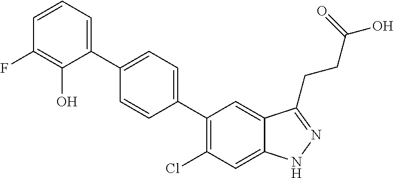 [SMILES: O=C(O)CCc1n[nH]c2cc(Cl)c(-c3ccc(-c4cccc(F)c4O)cc3)cc12] | 3-(6-chloro-5-(3′- fluoro-2′-hydroxy- [1,1′-biphenyl]-4- yl)-1H-indazol-3- yl)propanoic acid | P7 | 411 Method C 2.893 min |
| 23 | 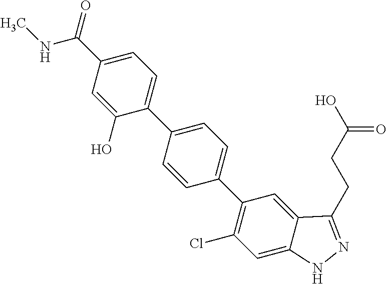 [SMILES: CNC(=O)c1ccc(-c2ccc(-c3cc4c(CCC(=O)O)n[nH]c4cc3Cl)cc2)c(O)c1] | 3-(6-chloro-5-(2′- hydroxy-4′-(methyl- carbamoyl)-[1,1′- biphenyl]-4-yl)- 1H-indazol-3-yl)- propanoic acid | P7 | 450.1 Method A 0.72 min |
| 24 | 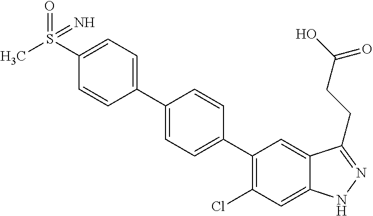 [SMILES: CS(=N)(=O)c1ccc(-c2ccc(-c3cc4c(CCC(=O)O)n[nH]c4cc3Cl)cc2)cc1] | 3-(6-chloro-5-(4′- (S-methylsulfon- imidoyl)-[1,1′- biphenyl]-4-yl)- 1H-indazol-3-yl)- propanoic acid | P7 | 454.2 Method A 0.66 min |
| 25 | 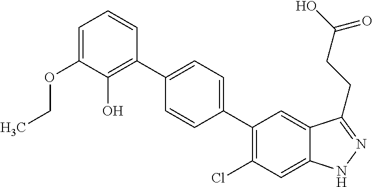 [SMILES: CCOc1cccc(-c2ccc(-c3cc4c(CCC(=O)O)n[nH]c4cc3Cl)cc2)c1O] | 3-(6-chloro-5-(3′- ethoxy-2′-hydroxy- [1,1′-biphenyl]-4- yl)-1H-indazol-3- yl)propanoic acid | P7 | 437 Method A 0.909 min |
| 26 | 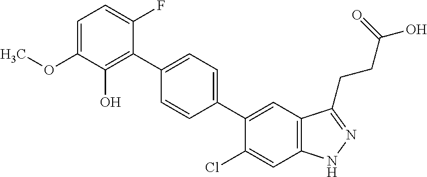 [SMILES: COc1ccc(F)c(-c2ccc(-c3cc4c(CCC(=O)O)n[nH]c4cc3Cl)cc2)c1O] | 3-(6-chloro-5-(6′- fluoro-2′-hydroxy- 3′-methoxy-[1,1′- biphenyl]-4-yl)- 1H-indazol-3-yl)- propanoic acid | P7 | ¹H NMR(400 MHz, MeOD) δ 7.85 (s, 1H), 7.62 (s, 1H), 7.49 (s, 4H), 6.91 (dd, 1H), 6.64 (t, 1H), 3.90 (s, 3H), 3.26 (t, 2H), 2.70 (t, 2H); MS (M + H)⁺ 441.0. |
| 27 | 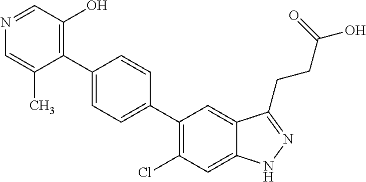 [SMILES: Cc1cncc(O)c1-c1ccc(-c2cc3c(CCC(=O)O)n[nH]c3cc2Cl)cc1] | 3-(6-chloro-5-(4- (3-hydroxy-5- methylpyridin-4- yl)phenyl)-1H- indazol-3-yl)- propanoic acid | P7 | 408 Method C 2.038 min |
| 28 | 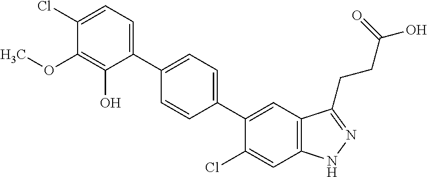 [SMILES: COc1c(Cl)ccc(-c2ccc(-c3cc4c(CCC(=O)O)n[nH]c4cc3Cl)cc2)c1O] | 3-(6-chloro-5-(4′- chloro-2′-hydroxy- 3′-methoxy-[1,1′- biphenyl]-4-yl)-1H- indazol-3-yl)- propanoic acid | P7 | ¹H NMR (400 MHz, MeOD) δ 7.82 (s, 1H), 7.63 (m, 3H), 7.49 (d, 2H), 7.10 (d, 1H), 6.95 (d, 1H), 3.88 (s, 3H), 3.27 (t, 2H), 2.73 (t, 2H); MS (M + H)⁺ 457.0. |
| 29 | 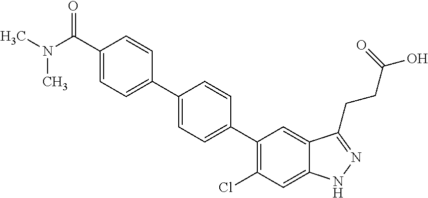 [SMILES: CN(C)C(=O)c1ccc(-c2ccc(-c3cc4c(CCC(=O)O)n[nH]c4cc3Cl)cc2)cc1] | 3-(6-chloro-5-(4′- (dimethylcarbam- oyl)-[1,1′-biphenyl]- 4-yl)-1H-indazol-3- yl)propanoic acid | P7 | 448 Method C 2.608 min |
| 30 | 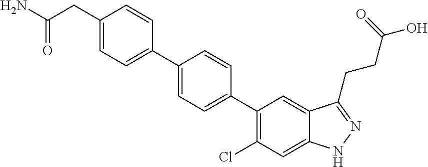 [SMILES: NC(=O)Cc1ccc(-c2ccc(-c3cc4c(CCC(=O)O)n[nH]c4cc3Cl)cc2)cc1] | 3-(5-(4′-(2-amino- 2-oxoethyl)-[1,1′- biphenyl]-4-yl)-6- chloro-1H-indazol- 3-yl)propanoic acid | P7 | 434 Method C 2.405 min |
| 32 | 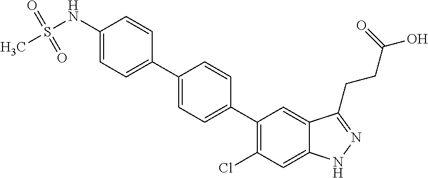 [SMILES: CS(=O)(=O)Nc1ccc(-c2ccc(-c3cc4c(CCC(=O)O)n[nH]c4cc3Cl)cc2)cc1] | 3-(6-chloro-5-(4′- (methylsulfon- amido)-[1,1′- biphenyl]-4-yl)- 1H-indazol-3-yl)- propanoic acid | P7 | 470 Method C 2.604 min |
| 33 | 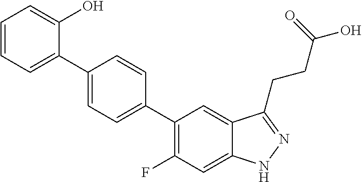 [SMILES: O=C(O)CCc1n[nH]c2cc(F)c(-c3ccc(-c4ccccc4O)cc3)cc12] | 3-(6-fluoro-5-(2′- hydroxy-[1,1′- biphenyl]-4-yl)- 1H-indazol-3-yl)- propanoic acid | P6, P11 | 377.4 Method G 1.96 min |
| 34 | 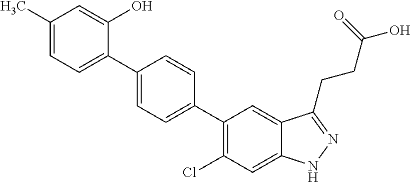 [SMILES: Cc1ccc(-c2ccc(-c3cc4c(CCC(=O)O)n[nH]c4cc3Cl)cc2)c(O)c1] | 3-(6-chloro-5-(2′- hydroxy-4′-methyl- [1,1′-biphenyl]-4- yl)-1H-indazol-3- yl)propanoic acid | P7 | 407 Method C 2.746 min |
| 35 | 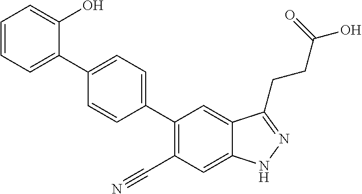 [SMILES: N#Cc1cc2[nH]nc(CCC(=O)O)c2cc1-c1ccc(-c2ccccc2O)cc1] | 3-(6-cyano-5-(2′- hydroxy-[1,1′- biphenyl]-4-yl)- 1H-indazol-3-yl)- propanoic acid | P6, P10 | 384.2 Method G 2.52 min. |
| 36 | 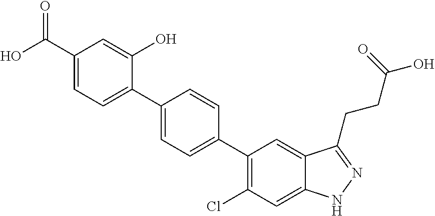 [SMILES: O=C(O)CCc1n[nH]c2cc(Cl)c(-c3ccc(-c4ccc(C(=O)O)cc4O)cc3)cc12] | 4′-(3-(2-carboxy- ethyl)-6-chloro- 1H-indazol-5-yl)- 2-hydroxy-[1,1′- biphenyl]-4- carboxylic acid | P7 | 437.3 Method A 0.556 min |
| 37 | 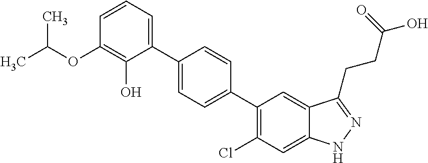 [SMILES: CC(C)Oc1cccc(-c2ccc(-c3cc4c(CCC(=O)O)n[nH]c4cc3Cl)cc2)c1O] | 3-(6-chloro-5-(2′- hydroxy-3′-iso- propoxy-[1,1′- biphenyl]-4-yl)- 1H-indazol-3-yl)- propanoic acid | P7 | 451.4 Method A 0.913 min |
| 38 | 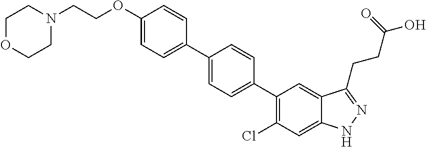 [SMILES: O=C(O)CCc1n[nH]c2cc(Cl)c(-c3ccc(-c4ccc(OCCN5CCOCC5)cc4)cc3)cc12] | 3-(6-chloro-5-(4′- (2-morpholinoeth- oxy)-[1,1′-biphen- yl]-4-yl)-1H-ind- azol-3-yl)propanoic acid | P7 | 506 Method B 2.496 min |
| 39 | 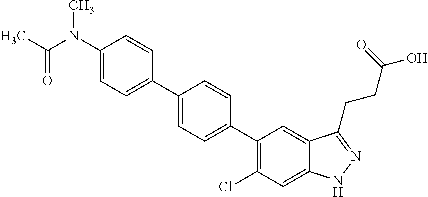 [SMILES: CC(=O)N(C)c1ccc(-c2ccc(-c3cc4c(CCC(=O)O)n[nH]c4cc3Cl)cc2)cc1] | 3-(6-chloro-5-(4′- (N-methylacet- amido)-[1,1′- biphenyl]-4-yl)- 1H-indazol-3-yl)- propanoic acid | P7 | 448 Method C 2.625 min |
| 40 | 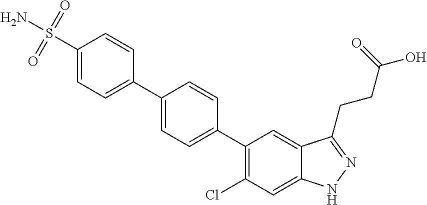 [SMILES: NS(=O)(=O)c1ccc(-c2ccc(-c3cc4c(CCC(=O)O)n[nH]c4cc3Cl)cc2)cc1] | 3-(6-chloro-5-(4′- sulfamoyl-[1,1′- biphenyl]-4-yl)- 1H-indazol-3-yl)- propanoic acid | P7 | 456 Method C 2.465 min |
| 41 | 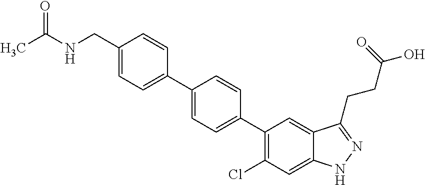 [SMILES: CC(=O)NCc1ccc(-c2ccc(-c3cc4c(CCC(=O)O)n[nH]c4cc3Cl)cc2)cc1] | 3-(5-(4′-(acetamido- methyl)-[1,1′- biphenyl]-4-yl)-6- chloro-1H-indazol- 3-yl)propanoic acid | P7 | 448 Method C 2.494 min |
| 42 | 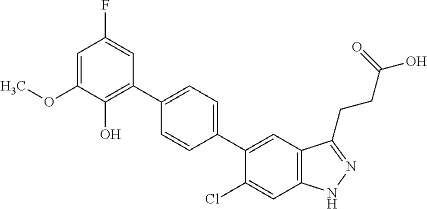 [SMILES: COc1cc(F)cc(-c2ccc(-c3cc4c(CCC(=O)O)n[nH]c4cc3Cl)cc2)c1O] | 3-(6-chloro-5-(5′- fluoro-2′-hydroxy- 3′-methoxy-[1,1′- biphenyl]-4-yl)- 1H-indazol-3-yl)- propanoic acid | P7 | 441.1 Method A 0.821 min |
| 43 | 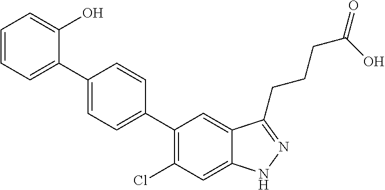 [SMILES: O=C(O)CCCc1n[nH]c2cc(Cl)c(-c3ccc(-c4ccccc4O)cc3)cc12] | 4-(6-chloro-5-(2′- hydroxy-[1,1′- biphenyl]-4-yl)- 1H-indazol-3-yl)- butanoic acid | P31, P6 | 407.3 Method A 0.905 min |
| 44 | 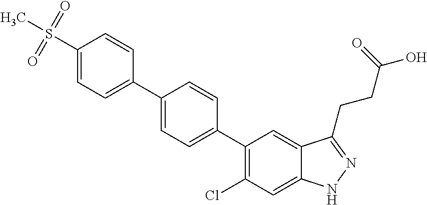 [SMILES: CS(=O)(=O)c1ccc(-c2ccc(-c3cc4c(CCC(=O)O)n[nH]c4cc3Cl)cc2)cc1] | 3-(6-chloro-5-(4′- (methylsulfonyl)- [1,1′-biphenyl]-4- yl)-1H-indazol-3- yl)propanoic acid | P7 | 455.3 Method A 0.735 min |
| 45 | 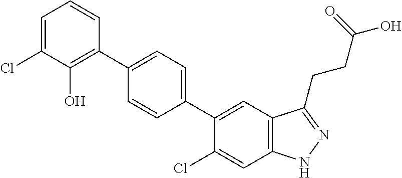 [SMILES: O=C(O)CCc1n[nH]c2cc(Cl)c(-c3ccc(-c4cccc(Cl)c4O)cc3)cc12] | 3-(6-chloro-5-(3′- chloro-2′-hydroxy- [1,1′-biphenyl]-4- yl)-1H-indazol-3- yl)propanoic acid | P7 | 427 Method C 3.071 min |
| 46 | 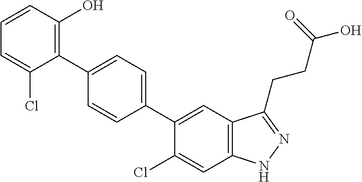 [SMILES: O=C(O)CCc1n[nH]c2cc(Cl)c(-c3ccc(-c4c(O)cccc4Cl)cc3)cc12] | 3-(6-chloro-5-(2′- chloro-6′-hydroxy- [1,1′-biphenyl]-4- yl)-1H-indazol-3- yl)propanoic acid | P7 | 427 Method C 2.928 min |
| 47 | 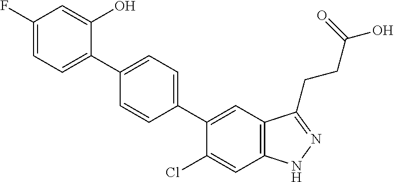 [SMILES: O=C(O)CCc1n[nH]c2cc(Cl)c(-c3ccc(-c4ccc(F)cc4O)cc3)cc12] | 3-(6-chloro-5-(4′- fluoro-2′-hydroxy- [1,1′-biphenyl]-4- yl)-1H-indazol-3- yl)propanoic acid | P7 | 411 Method C 2.907 min |
| 48 | 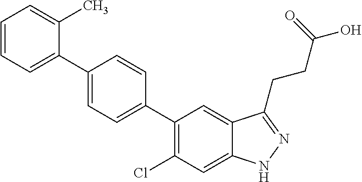 [SMILES: Cc1ccccc1-c1ccc(-c2cc3c(CCC(=O)O)n[nH]c3cc2Cl)cc1] | 3-(6-chloro-5-(2′- methyl-[1,1′- biphenyl]-4-yl)- 1H-indazol-3-yl)- propanoic acid | P7 | 391 Method D 2.739 min |
| 49 | 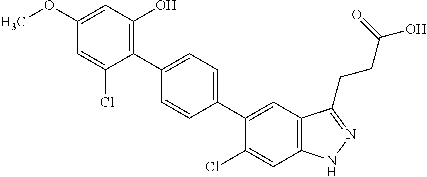 [SMILES: COc1cc(O)c(-c2ccc(-c3cc4c(CCC(=O)O)n[nH]c4cc3Cl)cc2)c(Cl)c1] | 3-(6-chloro-5-(2′- chloro-6′-hydroxy- 4′-methoxy-[1,1′- biphenyl]-4-yl)-1H- indazol-3-yl)- propanoic acid | P7 | 457 Method C 2.932 min |
| 50 | 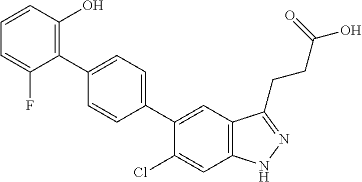 [SMILES: O=C(O)CCc1n[nH]c2cc(Cl)c(-c3ccc(-c4c(O)cccc4F)cc3)cc12] | 3-(6-chloro-5-(2′- fluoro-6′-hydroxy- [1,1′-biphenyl]-4- yl)-1H-indazol-3- yl)propanoic acid | P7 | 411 Method C 2.807 min |
| 51 | 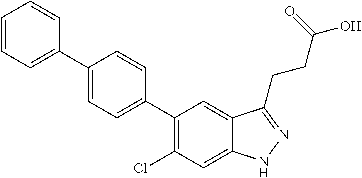 [SMILES: O=C(O)CCc1n[nH]c2cc(Cl)c(-c3ccc(-c4ccccc4)cc3)cc12] | 3-(5-([1,1′-biphen- yl]-4-yl)-6-chloro- 1H-indazol-3-yl)- propanoic acid | P7 | 377.3 Method A 0.895 min |
| 52 | 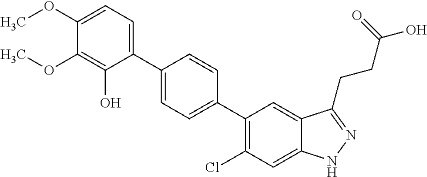 [SMILES: COc1ccc(-c2ccc(-c3cc4c(CCC(=O)O)n[nH]c4cc3Cl)cc2)c(O)c1OC] | 3-(6-chloro-5-(2′- hydroxy-3′,4′- dimethoxy-[1,1′- biphenyl]-4-yl)-1H- indazol-3-yl)- propanoic acid | P7 | 453 Method C 2.887 min |
| 53 | 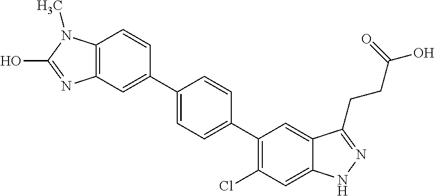 [SMILES: Cn1c(O)nc2cc(-c3ccc(-c4cc5c(CCC(=O)O)n[nH]c5cc4Cl)cc3)ccc21] | 3-(6-chloro-5-(4-(2- hydroxy-1-methyl- 1H-benzo[d]imid- azol-5-yl)phenyl)- 1H-indazol-3-yl)- propanoic acid | P7 | 447 Method C 2.506 min |
| 54 | 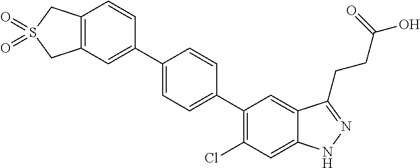 [SMILES: O=C(O)CCc1n[nH]c2cc(Cl)c(-c3ccc(-c4ccc5c(c4)CS(=O)(=O)C5)cc3)cc12] | 3-(6-chloro-5-(4- (2,2-dioxido-1,3- dihydrobenzo[c]- thiophen-5-yl)- phenyl)-1H-indazol- 3-yl)propanoic acid | P7 | 467 Method B 2.844 min |
| 55 | 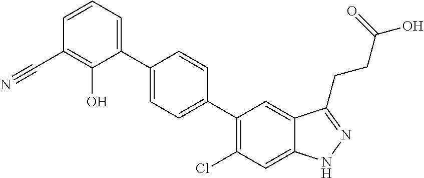 [SMILES: N#Cc1cccc(-c2ccc(-c3cc4c(CCC(=O)O)n[nH]c4cc3Cl)cc2)c1O] | 3-(6-chloro-5-(3′- cyano-2′-hydroxy- [1,1′-biphenyl]-4- yl)-1H-indazol-3- yl)propanoic acid | P7 | 418 Method C 2.842 min |
| 56 | 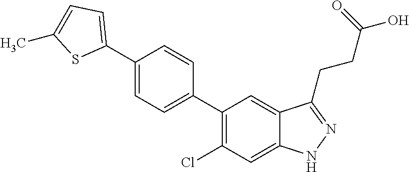 [SMILES: Cc1ccc(-c2ccc(-c3cc4c(CCC(=O)O)n[nH]c4cc3Cl)cc2)s1] | 3-(6-chloro-5-(4- (5-methylthiophen- 2-yl)phenyl)-1H- indazol-3-yl)- propanoic acid | P7 | 397 Method D 2.745 min |
| 57 | 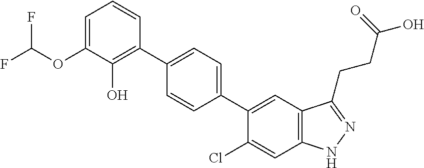 [SMILES: O=C(O)CCc1n[nH]c2cc(Cl)c(-c3ccc(-c4cccc(OC(F)F)c4O)cc3)cc12] | 3-(6-chloro-5-(3′- (difluoromethoxy)- 2′-hydroxy-[1,1′- biphenyl]-4-yl)- 1H-indazol-3-yl)- propanoic acid | P7 | 459.1 Method A 0.841 min |
| 58 | 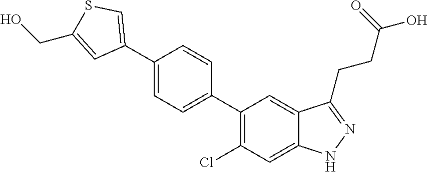 [SMILES: O=C(O)CCc1n[nH]c2cc(Cl)c(-c3ccc(-c4csc(CO)c4)cc3)cc12] | 3-(6-chloro-5-(4-(5- (hydroxymethyl)- thiophen-3-yl)phen- yl)-1H-indazol-3- yl)propanoic acid | P7 | 413 Method C 2.581 min |
| 59 | 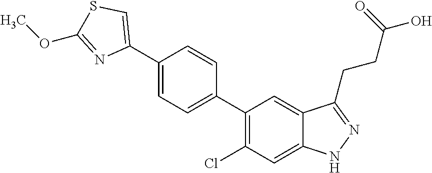 [SMILES: COc1nc(-c2ccc(-c3cc4c(CCC(=O)O)n[nH]c4cc3Cl)cc2)cs1] | 3-(6-chloro-5-(4- (2-methoxythiazol- 4-yl)phenyl)-1H- indazol-3-yl)- propanoic acid | P7 | 414 Method C 3.051 min |
| 60 | 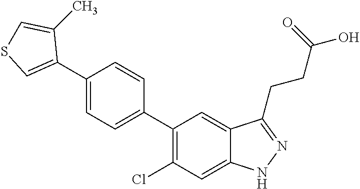 [SMILES: Cc1cscc1-c1ccc(-c2cc3c(CCC(=O)O)n[nH]c3cc2Cl)cc1] | 3-(6-chloro-5-(4-(4- methylthiophen-3- yl)phenyl)-1H- indazol-3-yl)- propanoic acid | P7 | 397 Method D 2.656 min |
| 61 | 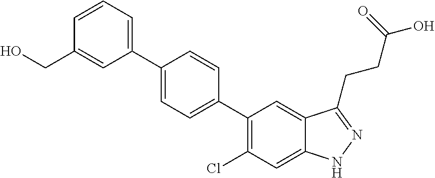 [SMILES: O=C(O)CCc1n[nH]c2cc(Cl)c(-c3ccc(-c4cccc(CO)c4)cc3)cc12] | 3-(6-chloro-5-(3′- (hydroxymethyl)- [1,1′-biphenyl]-4- yl)-1H-indazol-3- yl)propanoic acid | P7 | 407 Method E 2.1 min |
| 62 | 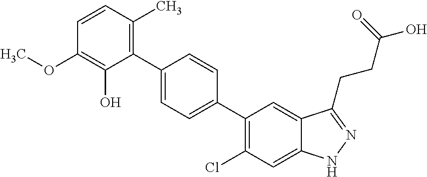 [SMILES: COc1ccc(C)c(-c2ccc(-c3cc4c(CCC(=O)O)n[nH]c4cc3Cl)cc2)c1O] | 3-(6-chloro-5-(2′- hydroxy-3′-meth- oxy-6′-methyl-[1,1′- biphenyl]-4-yl)-1H- indazol-3-yl)- propanoic acid | P7 | 437 Method A 0.865 min |
| 63 | 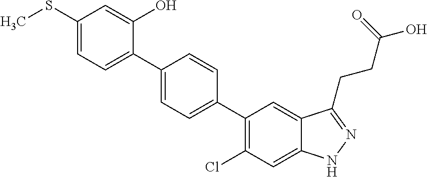 [SMILES: CSc1ccc(-c2ccc(-c3cc4c(CCC(=O)O)n[nH]c4cc3Cl)cc2)c(O)c1] | 3-(6-chloro-5-(2′- hydroxy-4′-(methyl- thio)-[1,1′-biphen- yl]-4-yl)-1H-indazol- 3-yl)propanoic acid | P7 | 439.2 Method A 0.871 min |
| 64 | 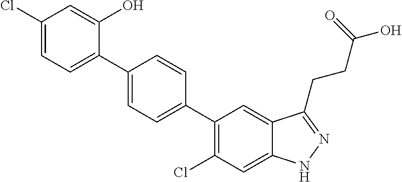 [SMILES: O=C(O)CCc1n[nH]c2cc(Cl)c(-c3ccc(-c4ccc(Cl)cc4O)cc3)cc12] | 3-(6-chloro-5-(4′- chloro-2′-hydroxy- [1,1′-biphenyl]-4- yl)-1H-indazol-3- yl)propanoic acid | P7 | 427 Method D 2.508 min |
| 65 | 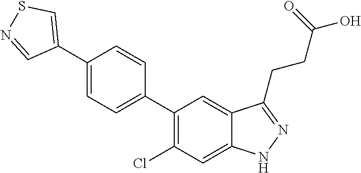 [SMILES: O=C(O)CCc1n[nH]c2cc(Cl)c(-c3ccc(-c4cnsc4)cc3)cc12] | 3-(6-chloro-5-(4- (isothiazol-4-yl)- phenyl)-1H-indazol- 3-yl)propanoic acid | P7 | 384 Method C 2.739 min |
| 66 | 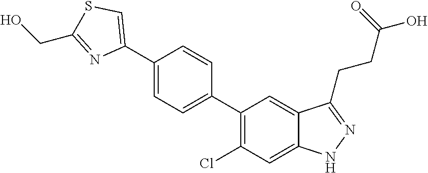 [SMILES: O=C(O)CCc1n[nH]c2cc(Cl)c(-c3ccc(-c4csc(CO)n4)cc3)cc12] | 3-(6-chloro-5-(4-(2- (hydroxymethyl)- thiazol-4-yl)phenyl)- 1H-indazol-3-yl)- propanoic acid | P7 | 414 Method B 2.614 min |
| 67 | 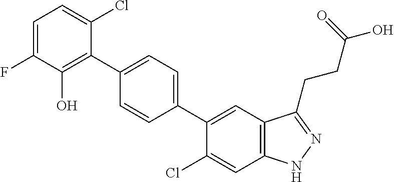 [SMILES: O=C(O)CCc1n[nH]c2cc(Cl)c(-c3ccc(-c4c(Cl)ccc(F)c4O)cc3)cc12] | 3-(6-chloro-5-(6′- chloro-3′-fluoro-2′- hydroxy-[1,1′- biphenyl]-4-yl)-1H- indazol-3-yl)- propanoic acid | P7 | 455.2 Method A 0.833 min |
| 68 | 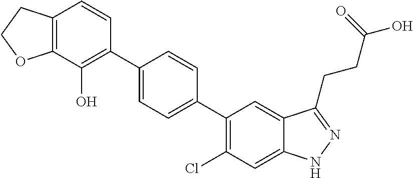 [SMILES: O=C(O)CCc1n[nH]c2cc(Cl)c(-c3ccc(-c4ccc5c(c4O)OCC5)cc3)cc12] | 3-(6-chloro-5-(4- (7-hydroxy-2,3- dihydrobenzofuran- 6-yl)phenyl)-1H- indazol-3-yl)- propanoic acid | P7 | 435.3 Method A 0.793 min |
| 69 | 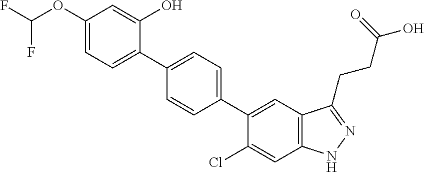 [SMILES: O=C(O)CCc1n[nH]c2cc(Cl)c(-c3ccc(-c4ccc(OC(F)F)cc4O)cc3)cc12] | 3-(6-chloro-5-(4′- (difluoromethoxy)- 2′-hydroxy-[1,1′- biphenyl]-4-yl)-1H- indazol-3-yl)- propanoic acid | P7 | 459.1 Method A 0.85 min |
| 70 | 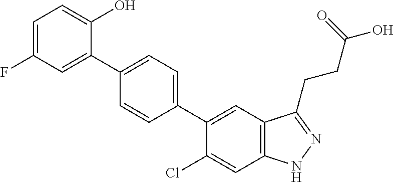 [SMILES: O=C(O)CCc1n[nH]c2cc(Cl)c(-c3ccc(-c4cc(F)ccc4O)cc3)cc12] | 3-(6-chloro-5-(5′- fluoro-2′-hydroxy- [1,1′-biphenyl]-4- yl)-1H-indazol-3- yl)propanoic acid | P7 | 411 Method C 2.873 min |
| 71 | 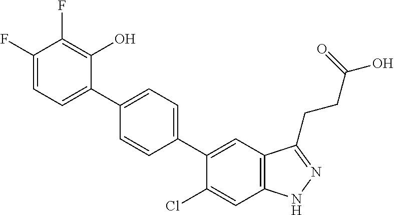 [SMILES: O=C(O)CCc1n[nH]c2cc(Cl)c(-c3ccc(-c4ccc(F)c(F)c4O)cc3)cc12] | 3-(6-chloro-5-(3′,4′- difluoro-2′-hydroxy- [1,1′-biphenyl]-4-yl)- 1H-indazol-3-yl)- propanoic acid | P7 | 429.1 Method A 0.836 min |
| 72 | 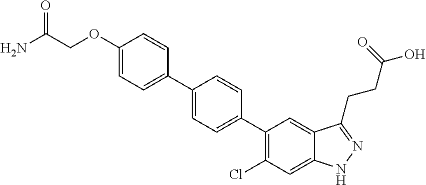 [SMILES: NC(=O)COc1ccc(-c2ccc(-c3cc4c(CCC(=O)O)n[nH]c4cc3Cl)cc2)cc1] | 3-(5-(4′-(2-amino- 2-oxoethoxy)-[1,1′- biphenyl]-4-yl)-6- chloro-1H-indazol- 3-yl)propanoic acid | P7 | 450 Method C 2.46 min |
| 73 | 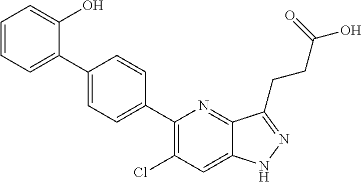 [SMILES: O=C(O)CCc1n[nH]c2cc(Cl)c(-c3ccc(-c4ccccc4O)cc3)nc12] | 3-(6-chloro-5-(2′- hydroxy-[1,1′- biphenyl]-4-yl)-1H- pyrazolo[4,3-b]- pyridin-3-yl)- propanoic acid | P7 | 394.2 Method G 2.59 min |
| 74 | 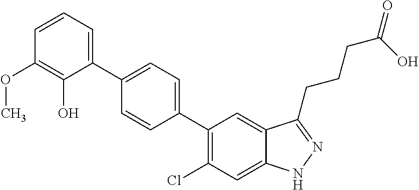 [SMILES: COc1cccc(-c2ccc(-c3cc4c(CCCC(=O)O)n[nH]c4cc3Cl)cc2)c1O] | 4-(6-chloro-5-(2′- hydroxy-3′-methoxy- [1,1′-biphenyl]-4-yl)- 1H-indazol-3-yl)- butanoic acid | P31, P6 | 437.3 Method A 0.941 min |
| 75 | 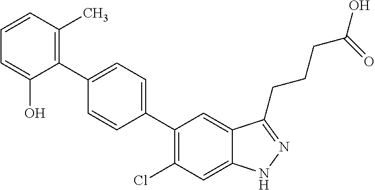 [SMILES: Cc1cccc(O)c1-c1ccc(-c2cc3c(CCCC(=O)O)n[nH]c3cc2Cl)cc1] | 4-(6-chloro-5-(2′- hydroxy-6′-methyl- [1,1′-biphenyl]-4- yl)-1H-indazol-3- yl)butanoic acid | P31, P6 | 421.2 Method A 0.899 min |
| 76 | 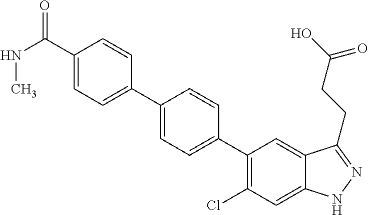 [SMILES: CNC(=O)c1ccc(-c2ccc(-c3cc4c(CCC(=O)O)n[nH]c4cc3Cl)cc2)cc1] | 3-(6-chloro-5-(4′- (methylcarbamoyl)- [1,1′-biphenyl]-4- yl)-1H-indazol-3- yl)propanoic acid | P7 | 434 Method F 1.96 min |
| 77 | 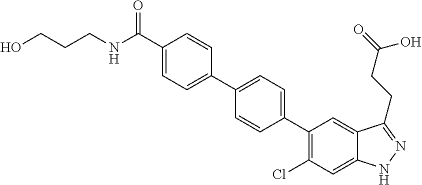 [SMILES: O=C(O)CCc1n[nH]c2cc(Cl)c(-c3ccc(-c4ccc(C(=O)NCCCO)cc4)cc3)cc12] | 3-(6-chloro-5-(4′- ((3-hydroxypropyl)- carbamoyl)-[1,1′- biphenyl]-4-yl)- 1H-indazol-3-yl)- propanoic acid | P7 | 478 Method C 2.368 min |
| 78 | 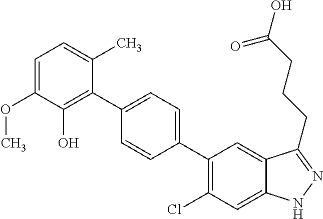 [SMILES: COc1ccc(C)c(-c2ccc(-c3cc4c(CCCC(=O)O)n[nH]c4cc3Cl)cc2)c1O] | 4-(6-chloro-5-(2′- hydroxy-3′-meth- oxy-6′-methyl-[1,1′- biphenyl]-4-yl)- 1H-indazol-3-yl)- butanoic acid | P31, P6 | 451.2 Method A 0.952 min |

### Biosynthesis of Glucuronide Metabolite Compounds

The compound of Example 1 (50 UM) was incubated with mouse liver microsomes (2 mg/mL) containing 10 μg/mL alamethicin, 3.3 mM MgCl2, and 5 mM uridine diphosphate glucuronic acid in a total volume of 40 mL KH₂PO₄ (100 mM, PH 7.4). The incubation was conducted in a 250-mL Erlenmeyer flask in a shaking water bath maintained at 37° C. for 60 hours. At the end of the incubation, the incubate was split into n=2, 20 mL aliquots, and 20 mL of CH₃CN was added to each sample, and the mixture was transferred to two 50 mL-polypropylene conical tubes and vigorously mixed on a vortex mixer. The tubes were spun in a Beckman centrifuge at 1800 g for 10 minutes, and the supernatant was transferred to new 50-mL polypropylene conical tubes. The tubes were subjected to vacuum centrifugation in a Genevac evaporator set at the HPLC mixture setting for approximately 4 hours to remove the CH₃CN. To the remaining solution, 10 mL of 0.1% formic acid in water: acetonitrile (95:5, % v: v). was added, and the resulting mixture was subjected to centrifugation in a Beckmann centrifuge at 40,000 g for 30 minutes to clarify the supernatant.

The supernatants were combined and transferred to a 50-mL polypropylene conical tube and directly applied onto a Varian Polaris C18 column (4.6×250 mm; 5 mm particle size) through a Jasco HPLC pump at a flow rate of 0.8 mL/min. After application of the 20 mL, another 5 mL of 0.1% formic acid in water:acetonitrile (95:5, % v:v) was pumped onto the column to ensure that the HPLC lines were cleared of the supernatant. The HPLC column was then transferred to a Agilent 1200 HPLC-UV system. The effluent was directly collected into PAS HTS-xt fraction collector. The mobile phase used consisted of 0.1% formic acid (mobile phase A) and CH₃CN (mobile phase B) at a flow rate of 0.5 ml/min. The mobile phase composition commenced at 95% A/5% B, was held at that composition for 5 minutes, followed by a linear gradient to 5% A/95% B at 45 minutes and held until 48 minutes, returned to initial conditions at 60 minutes. The mobile phase program and data collection were started by making a dummy injection of water from the autosampler. Fractions were collected every 7 seconds into a wide-welled polypropylene microtiter plate. Fractions collected in the region of interest (i.e., that where the UV detector showed the presence of significant absorbance) were injected (5 mL) onto a second HPLC gradient method using the same mobile phases with a Phenomenex XB-C18, 2.1×100 mm, 1.7u column at 0.4 mL/min flowrate to test for purity. The mobile phase composition commenced at 95% A/5% B, was held at that composition for 0.5 minutes, followed by a linear gradient to 5% A/95% B at 3.75 minutes and held until 4 minutes, returned to initial conditions at 5 minutes. Those containing the product of interest were combined into a 15-mL conical glass tube, and the solvent was evaporated by vacuum centrifugation in the Genevac evaporator. Upon dryness, they were prepared for NMR analysis.

For the purposes of structural characterization, samples were dissolved in 0.045 mL of deuterated methanol—MeOD “100%” (Cambridge Isotope Laboratories, Andover, MA) and placed in a 1.7 mm NMR tube in a dry argon atmosphere. For quantitative NMR, after structural characterization was completed, samples were dried and reconstituted in 0.045 mL of dimethyl sulfoxide—DMSO “100%” (Cambridge Isotope Laboratories, Andover, MA) and placed in a 1.7 mm NMR tube in a dry argon atmosphere. ¹H and ¹³C spectra were referenced using residual the residual solvent (DMSO-d6-¹H δ=2.50 ppm relative to TMS 6=0.00, ¹³C δ=39.50 ppm relative to TMS, 6=0.00, MeOD-d4-1H δ=3.35 ppm relative to TMS, 6=0.00, 13C δ=49.3 ppm relative to TMS, 6=0.00). NMR spectra were recorded on a Bruker Avance 600 MHz (Bruker BioSpin Corporation, Billerica, MA) controlled by Topspin V4.0 and equipped with a 1.7 mm TCI Cryo probe. 1D spectra were recorded using an approximate sweep width of 8400 Hz and a total recycle time of approximately 7 s. The resulting time-averaged free induction decays were transformed using an exponential line broadening of 1.0 Hz to enhance signal to noise. The 2D data were recorded using the standard pulse sequences provided by Bruker. At minimum a 1K×128 data matrix was acquired using a minimum of 2 scans and 16 dummy scans with a spectral width of 10000 Hz in the f2 dimension. The 2D data sets were zero-filled to at least 1 k data point. Post-acquisition data processing was performed with MestReNova V12.1. All conditions for the acquisition of these data are contained in the raw file. Quantitation was achieved using an external standard of 5 mM Benzoic Acid (Cambridge Isotope Laboratories, Andover, MA) and the quantitation plugin in Mnova software.

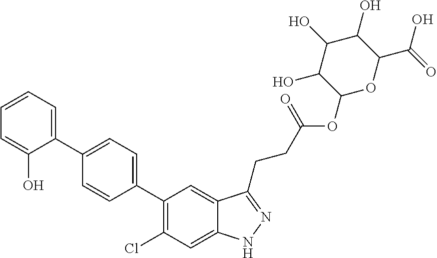 [SMILES: O=C(CCc1n[nH]c2cc(Cl)c(-c3ccc(-c4ccccc4O)cc3)cc12)OC1OC(C(=O)O)C(O)C(O)C1O]

### 6-((3-(6-chloro-5-(2′-hydroxy-[1,1′-biphenyl]-4-yl)-1H-indazol-3-yl)propanoyl)oxy)-3,4,5-trihydroxytetrahydro-2H-pyran-2-carboxylic acid (Example 79)

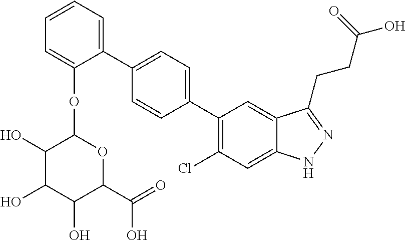 [SMILES: O=C(O)CCc1n[nH]c2cc(Cl)c(-c3ccc(-c4ccccc4OC4OC(C(=O)O)C(O)C(O)C4O)cc3)cc12]

### 6-((4′-(3-(2-carboxyethyl)-6-chloro-1H-indazol-5-yl)-[1,1′-biphenyl]-2-yl)oxy)-3,4,5-trihydroxytetrahydro-2H-pyran-2-carboxylic acid (Example 80)

### Expression and Purification of AMPK111

A tricistronic AMPK expression construct was prepared, which included open reading frames encoding the full-length α1, β1 and δ1 subunits of human AMPK with a ribosome-binding site (RBS) ahead of each coding region. The construct was subcloned into pET-14b expression vector (Novagen, Madison, Wisconsin) using standard molecular biology techniques. AMPK tricistronic construct was transformed into E. coli BL21-CodonPlus™ (DE3)-RIPL strain (Stratagene), and transformants were selected on LB (Luria-Bertani) agar plates containing ampicillin (100 μg/mL). Ten liters of LB medium (MP Biomedical LB broth #11-3002-032) containing 100 μg/mL carbenicillin was inoculated with 100 mL E. coli shake flask culture (BL-21, pET-14b, AMPK 111) in a BF4 10 L working volume bioreactor (New Brunswick Scientific Co.) at 37° C., 600 rpm, 6 L/minute aeration. Optical density sample measurements were made on an UltroSpec 2000 spectrophotometer (Pharmacia Biotech) at 600 nm.

When the cell density reached ˜0.9 OD, the temperature was reduced to 18° C. and the culture was induced at 18° C. with 0.1 mM Isopropylthiogalactoside (IPTG). The cell paste was collected at −18 hours post induction by refrigerated continuous flow centrifugation (Heraeus, rotor #8575) at 15,000 rpm at 4° C. The cell pellets were aliquoted into four portions, flash frozen in liquid nitrogen and were stored at −80° C. until purification. For purification, frozen cell paste was thawed and resuspended in 50 mL lysis buffer (50 mM Tris, pH 8.0, 150 mM NaCl, 10% glycerol, 2 mM Tris-2-carboxyethyl phosphine (TCEP), 20 mM imidazole and 0.001% Triton X-100). After sonication, insoluble material was removed by centrifugation at 15,000 rpm in a Sorvall® RC5 plus centrifuge for 30 min at 4° C. and the supernatant was loaded onto a 5 mL HisTrap™ HP column (GE Healthcare, Piscataway, NJ) and washed with five column volumes of lysis buffer. Bound proteins were eluted using an elution buffer containing 300 mM imidazole. Fractions containing AMPK subunits were pooled based on SDS-10% PAGE analysis and dialyzed overnight in dialysis buffer (50 mM Tris, pH 8.0, 150 mM NaCl, 10% glycerol, 2 mM TCEP, and 0.001% Triton X-100). The purified AMPK was phosphorylated on its activation loop Thr 172 by incubating 1.0 μM AMPK complex in the presence of 200 nM CaMKKB (calmodulin-dependent protein kinase B obtained from the University of Dundee) in phosphorylation buffer for 30 min at 30° C. The phosphorylated AMPK complex was re-purified on HisTrap™ HP column as before, dialyzed overnight in dialysis buffer. The phosphorylated AMPK complex was further purified by gel filtration chromatography with a Superdex 200 HiLoad 16/60 column (GE Healthcare) in SEC buffer (50 mM Tris, pH 8.0, 150 mM NaCl, 10% glycerol, 2 mM TCEP, and 0.001% Triton X-100). The final samples were stored at −20° C. with 25% glycerol.

### Expression and Purification of AMPK221

A tricistronic AMPK expression construct was designed that included open reading frames encoding the full-length α2, β2 and δ1 subunits of human AMPK with a ribosome-binding site (RBS) ahead of each coding region. The construct was subcloned into pET-14b expression vector (Novagen, Madison, Wisconsin) using standard molecular biology techniques. AMPK tricistronic construct was transformed into E. coli BL21-CodonPlus™ (DE3)-RIPL strain (Stratagene) and transformants were selected on LB (Luria-Bertani) agar plates containing ampicillin (100 μg/mL). Ten liters of LB medium (MP Biomedical LB broth #11-3002-032) containing 100 μg/mL carbenicillin was inoculated with 100 mL E. coli shake flask culture (BL-21, pET-14b, AMPK 221) in a BF4 10 L working volume bioreactor (New Brunswick Scientific Co.) at 37° C., 600 rpm, 6 L/minute aeration. Optical density sample measurements were made on an UltroSpec 2000 spectrophotometer (Pharmacia Biotech) at 600 nm.

When the cell density reached ˜0.9 OD, the temperature was reduced to 18° C. and the culture was induced at 18° C. with 0.1 mM Isopropylthiogalactoside (IPTG). The cell paste was collected at ˜18 hours post induction by refrigerated continuous flow centrifugation (Heraeus, rotor #8575) at 15,000 rpm at 4° C. The cell pellets were aliquoted into four portions, flash frozen in liquid nitrogen and were stored at −80° C. until purification. For purification, frozen cell paste was thawed and resuspended in 50 mL lysis buffer (50 mM Tris, pH 8.0, 150 mM NaCl, 10% glycerol, 2 mM Tris-2-carboxyethyl phosphine (TCEP), 20 mM imidazole and 0.001% Triton X-100). After sonication, insoluble material was removed by centrifugation at 15,000 rpm in a Sorvall® RC5 plus centrifuge for 30 min at 4° C. and the supernatant was loaded onto a 5 mL HisTrap™ HP column (GE Healthcare, Piscataway, NJ) and washed with five column volumes of lysis buffer. Bound proteins were eluted using an elution buffer containing 300 mM imidazole. Fractions containing AMPK subunits were pooled based on SDS-10% PAGE analysis and dialyzed overnight in dialysis buffer (50 mM Tris, pH 8.0, 150 mM NaCl, 10% glycerol, 2 mM TCEP, and 0.001% Triton X-100). The purified AMPK was phosphorylated on its activation loop Thr 172 by incubating 1.0 μM AMPK complex in the presence of 200 nM CaMKKB (calmodulin-dependent protein kinase B)obtained from the University of Dundee) in phosphorylation buffer for 30 min at 30° C. The phosphorylated AMPK complex was re-purified on HisTrap™ HP column as before, dialyzed overnight in dialysis buffer. The phosphorylated AMPK complex was further purified by gel filtration chromatography with a Superdex 200 HiLoad 16/60 column (GE Healthcare) in SEC buffer (50 mM Tris, pH 8.0, 150 mM NaCl, 10% glycerol, 2 mM TCEP, and 0.001% Triton X-100). The final samples were stored at −20° C. with 25% glycerol.

### Expression and Purification of PP2A

The coding sequence for human recombinant Protein Phosphatase 2A catalytic subunit (PPP2CA; 308 aa, P67775, AA2-309) with an N-terminal 2× FLAG tag and a TEV protease site was synthesized and subcloned into the pFastBac Dual expression vector (Thermo Fisher, 10712024) under the PoIH promoter and the Protein Phosphatase 2A regulatory subunit (PPP2R1A; 508 aa, P30153, AA2-589) with an N-terminal His tag. A TEV protease site was synthesized and subcloned into the same vector under the p10 promoter. The vector was used to make baculovirus using the Bac-to-Bac System (Thermo Fisher) which was subsequently used to express protein in Sf9 cells (Expression Systems, 94-001 F). The Sf9 cells were cultured in ESF 921 medium (Expression Systems, 96-001-01) at a 3 L volume in a sterile 5 L Thomson Optimum Growth Flask with a vent cap (Thomson Instrument Company, 931116) at 27° C. while shaking at 115 rpm with a 2-inch shaking diameter. P0 virus was used at 10 mis/L to infect cells at a density ˜2.5×106 vc/ml at >95% viability. The harvest time (71 hours post infection) was indicated by percent cell viability (-80%) and increased cell diameter (>3 micron). The cell paste was collected by centrifugation in a Thermo Sorvall RC 3BP+ Centrifuge at 5000×g and frozen at −80° C.

For purification, the 3 L of culture worth of cell paste was resuspended in 175 mL Lysis/wash buffer (50 mM Tris pH 8.0, 300 mM NaCl, 10% glycerol, 1 mM TCEP). Cells were lysed by micro fluidization at 15,000 PSI for 3 passes and clarified by centrifugation 30,000×g. 5 mL of FLAG resin was equilibrated with lysis buffer. Equilibrated FLAG resin was added to the supernatant and allowed to batch bind 6 hours. The protein was then washed with 20 volumes of lysis buffer and eluted off of the resin with 7 mL of elution buffer (50 mM Tris pH 8.0, 300 mM NaCl, 10% glycerol, 0.25 mg/ml FLAG peptide. Eluted fractions were analyzed by SDS-PAGE, Mass Spec, and PP2a activity. Total protein concentration was determined by Superdex 200 16-60, protein determined to be 0.149 mg/mL.

### Biochemical Profiling of AMPK Activators by AMPK111

The biochemical EC₅₀ (half-maximal concentration required for full activation) of compounds for the activation of AMPK was evaluated by HTRF assay using LANCE Ultra ULight-Acetyl-CoA Carboxylase (SAMS) peptide (commercially available, Perkin Elmer catalog TRF0133-M). 5 μL of 0.3 nM phosphorylated AMPK 111 (isolation detailed above) diluted in assay buffer (50 mM HEPES, 1 mM EGTA, 10 mM MgCl2, 0.25 mM DTT, 0.01% Tween-20, 0.01% BSA (pH 7.5), was added to white 384 well plates (Corning catalog number 3824) containing 0.075 μL of test compound, (solubilized and serially diluted in DMSO, in a 11 point, ½-log dilution series, tested in duplicate).

Plates were spun at 1000 RPM for 10 seconds. Following a fifteen-minute room temperature incubation, 5 μL of 30 nM protein phosphatase PP2A (isolation detailed above) diluted in the assay buffer was added to the plate, to dephosphorylate pThr172 of AMPK. Plates were spun at 1000 RPM for 10 seconds. After incubation for 120 minutes, 5 μL of substrate mixture containing 60 nM okadaic acid (Tocris catalog number 1136), 150 nM SAMS peptide (Perkin Elmer catalog TRF0133-M), and 60 μM ATP (Teknova catalog number A1204) diluted in assay buffer, was added to the plate. Plates were spun at 1000 RPM for 10 seconds. The reaction was terminated after 60 minutes incubation at room temperature, by the addition of 5 μL of stop and detection cocktail, which consisted of 1× Perkin Elmer Lance buffer (Perkin Elmer catalog number CR97-100), 40 mM EDTA (Thermo Fisher catalog number BP2482-100), and 2 nM Eu-anti-Acetyl CoA Carboxylase [pSer70] antibody (Perkin Elmer, TRF0208-M). Plates were spun at 1000 RPM for 10 seconds. Plates were incubated for 1 hour, then read on an Envision reader with settings for TR-FRET Ratio=10,000× (fluorescence intensity 665 nM/fluorescence intensity 615 nM). EC₅₀ values were determined from this data using a 4-parameter fit algorithm and are presented in Table 2.

### Biochemical Profiling of AMPK Activators by AMPK221

The biochemical EC₅₀ (half-maximal concentration required for full activation) of compounds for the activation of AMPK was evaluated by HTRF assay using LANCE Ultra ULight-Acetyl-CoA Carboxylase (SAMS) peptide (commercially available, Perkin Elmer catalog TRF0133-M). 5 μL of 0.3 nM phosphorylated AMPK 221 (isolation detailed above) diluted in assay buffer (50 mM HEPES, 1 mM EGTA, 10 mM MgCl2, 0.25 mM DTT, 0.01% Tween-20, 0.01% BSA (pH 7.5) was added to white 384 well plates (Corning catalog number 3824) containing 0.075 μL of test compound (solubilized and serially diluted in DMSO, in a 11 point, %-log series and tested in duplicate).

Plates were spun at 1000 RPM for 10 seconds. Following a fifteen-minute room temperature incubation, 5 μL of 15 nM protein phosphatase PP2A (isolation detailed above) diluted in the assay buffer was added to the plate to dephosphorylate pThr172 of AMPK. Plates were spun at 1000 RPM for 10 seconds. After incubation for 120 minutes, 5 μL of substrate mixture containing 30 nM okadaic acid (Tocris catalog number 1136), 150 nM SAMS peptide (Perkin Elmer catalog TRF0133-M) and 240 μM ATP (Teknova catalog number A1204), diluted in assay buffer, was added to the plate. Plates were spun at 1000 RPM for 10 seconds. The reaction was terminated after 60 minutes incubation at room temperature by the addition of 5 μL of stop and detection cocktail, which consisted of 1× Perkin Elmer Lance buffer (Perkin Elmer catalog number CR97-100), 40 mM EDTA (Thermo Fisher catalog number BP2482-100), and 2 nM Eu-anti-Acetyl CoA Carboxylase [pSer70] antibody (Perkin Elmer, TRF0208-M). Plates were spun at 1000 RPM, 10 seconds. Plates were incubated for 1 hour, then read on an Envision reader with settings for TR-FRET Ratio=10,000× (fluorescence intensity 665 nM/fluorescence intensity 615 nM). EC₅₀ values were determined from this data using a 4-parameter fit algorithm and are presented in Table 2.

TABLE 2
Biochemical profiling of AMPK activators
by AMPK 111 and AMPK221

| | |
|---|---|
| AMPK 111 EC₅₀ | AMPK 221 |

| Example | nM | eMax (%) | nM | eMax (%) |
|---|---|---|---|---|
| 1 | 47 | 75 | 88 | 57 |
| 2 | 29 | 80 | 34 | 66 |
| 3 | 189 | 83 | 528 | 61 |
| 4 | 62 | 74 | 665 | 34 |
| 5 | 89 | 59 | 37 | 62 |
| 6 | 6 | 64 | 4 | 53 |
| 7 | 90 | 71 | 359 | 57 |
| 8 | 26 | 75 | 38 | 56 |
| 9 | 11 | 90 | 41 | 68 |
| 10 | 29 | 76 | 48 | 55 |
| 11 | 53 | 62 | 60 | 44 |
| 12 | 16 | 72 | 62 | 56 |
| 13 | 21 | 86 | 69 | 67 |
| 14 | 45 | 74 | 72 | 65 |
| 15 | 35 | 75 | 76 | 52 |
| 16 | 37 | 86 | 81 | 64 |
| 17 | 34 | 80 | 83 | 49 |
| 18 | 12 | 84 | 96 | 64 |
| 19 | 54 | 79 | 144 | 34 |
| 20 | 16 | 82 | 97 | 61 |
| 21 | 151 | 65 | 97 | 56 |
| 22 | 111 | 74 | 100 | 59 |
| 23 | 2 | 71 | 5 | 56 |
| 24 | 19 | 86 | 106 | 48 |
| 25 | 144 | 71 | 107 | 61 |
| 26 | 107 | 77 | 114 | 66 |
| 27 | 7 | 87 | 125 | 62 |
| 28 | 277 | 73 | 136 | 63 |
| 29 | 46 | 72 | 162 | 63 |
| 30 | 36 | 80 | 173 | 65 |
| 31 | 57 | 84 | 190 | 61 |
| 32 | 136 | 71 | 198 | 41 |
| 33 | 301 | 57 | 196 | 57 |
| 34 | 228 | 68 | 197 | 50 |
| 35 | 137 | 67 | 198 | 47 |
| 36 | 45 | 68 | 200 | 49 |
| 37 | 464 | 70 | 205 | 52 |
| 38 | 95 | 67 | 209 | 64 |
| 39 | 109 | 69 | 218 | 59 |
| 40 | 92 | 85 | 306 | 68 |
| 41 | 70 | 66 | 235 | 65 |
| 42 | 291 | 68 | 244 | 64 |
| 43 | 51 | 80 | 248 | 62 |
| 44 | 85 | 79 | 204 | 52 |
| 45 | 123 | 82 | 263 | 66 |
| 46 | 20 | 82 | 267 | 59 |
| 47 | 113 | 83 | 282 | 59 |
| 48 | 365 | 43 | 367 | 9 |
| 49 | 96 | 74 | 325 | 61 |
| 50 | 153 | 76 | 337 | 55 |
| 51 | 312 | 71 | 355 | 22 |
| 52 | 6 | 85 | 19 | 65 |
| 53 | 218 | 69 | 644 | 55 |
| 54 | 217 | 61 | 1006 | 64 |
| 55 | 208 | 45 | 389 | 38 |
| 56 | 363 | 48 | 477 | 18 |
| 57 | 103 | 75 | 426 | 63 |
| 58 | 215 | 76 | 573 | 57 |
| 59 | 609 | 30 | 546 | 13 |
| 60 | 241 | 53 | 532 | 21 |
| 61 | 692 | 55 | 645 | 34 |
| 62 | 25 | 72 | 14 | 57 |
| 63 | 281 | 56 | 565 | 64 |
| 64 | 203 | 76 | 617 | 48 |
| 65 | 263 | 79 | 795 | 58 |
| 66 | 365 | 74 | 1087 | 63 |
| 67 | 69 | 85 | 953 | 63 |
| 68 | 29 | 71 | 19 | 69 |
| 69 | 480 | 73 | 1151 | 58 |
| 70 | 285 | 74 | 1086 | 49 |
| 71 | 279 | 73 | 994 | 60 |
| 72 | 99 | 103 | 1831 | 114 |
| 73 | 26 | 86 | 2115 | 30 |
| 74 | 92 | 80 | 66 | 69 |
| 75 | 74 | 75 | 229 | 63 |
| 76 | 6 | 67 | 13 | 53 |
| 77 | 70 | 66 | 82 | 58 |
| 78 | 93 | 78 | 87 | 66 |
| 79 | 39 | 67 | 29 | 27 |
| 80 | >21100 | | >21100 | |

### Crystal Form Analysis (Compound of Example 1, Form 1)

Powder X-ray diffraction (PXRD) analysis was conducted using a Bruker AXS D8 Endeavor diffractometer equipped with a Cu radiation source. The divergence slit was set at 15 mm continuous illumination. Diffracted radiation was detected by a PSD-Lynx Eye detector, with the detector PSD opening set at 4.111 degrees. The X-ray tube voltage and amperage were set to 40 kV and 40 mA respectively. In addition, the energy dispersive detector, a nickel filter was used to screen out unwanted wavelengths. Data was collected in the Theta-Theta goniometer at the Cu wavelength from 3.0 to 40.0 degrees 2-Theta using a step size of 0.0160 degrees and a step time of 1.0 second. The antiscatter screen was set to a fixed distance of 1.5 mm. Samples were rotated at 15/min during collection. Samples were prepared by placing them in a silicon low background sample holder and rotated during collection. Data were collected using Bruker DIFFRAC Plus software and analysis was performed by EVA diffract plus software. The sample holder used in a particular experiment is given by a codename within the filename: SD=small divot holder.

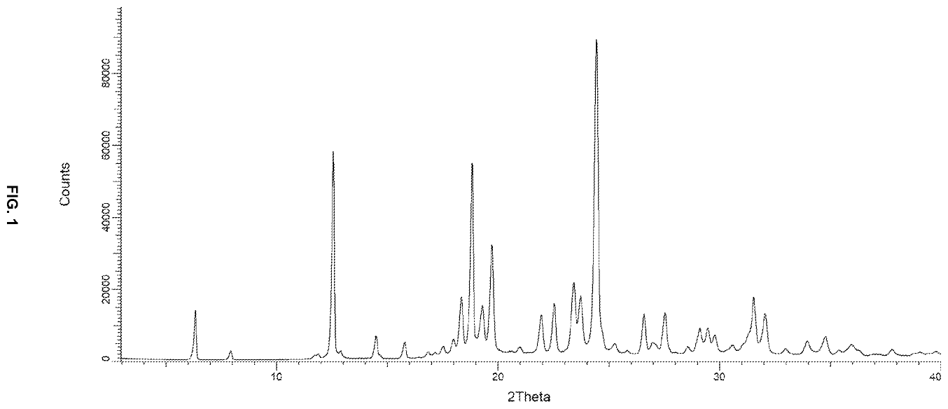

Samples were prepared by placing them in a silicon low background sample holder and rotated during collection. Data were collected using Bruker DIFFRAC Plus software and analysis was performed by EVA diffract plus software. The PXRD diffractogram is shown in FIG. 1. Using the peak search algorithm in the EVA software, peaks selected with a threshold value of 1 were used to make preliminary peak assignments. To ensure validity, adjustments were manually made; the output of automated assignments was visually checked and peak positions were adjusted to the peak maximum. Peaks with relative intensity of ≥3% were generally chosen. The peaks which were not resolved or were consistent with noise were not selected. A typical error associated with the peak position from PXRD stated in USP is up to +/−0.2° 2-Theta (USP-941). The peak list is shown in Table 3. Characteristic peaks of Form 1 are provided in Table 4. FIG. 1 shows the PXRD of the compound of Example 1, form 1.

TABLE 3
Peak list for Form 1, Example 1 using threshold ≥3%.

| Angle | Rel. Intensity |
|---|---|
| (°2-theta) | (%) |
| 6.3 | 15 |
| 7.9 | 3 |
| 12.6 | 64 |
| 14.5 | 7 |
| 15.8 | 5 |
| 17.5 | 3 |
| 18.0 | 5 |
| 18.3 | 18 |
| 18.8 | 61 |
| 19.3 | 15 |
| 19.7 | 34 |
| 22.0 | 12 |
| 22.5 | 16 |
| 23.4 | 22 |
| 23.7 | 18 |
| 24.4 | 100 |
| 25.3 | 3 |
| 26.6 | 13 |
| 27.0 | 3 |
| 27.1 | 3 |
| 27.5 | 13 |
| 29.1 | 8 |
| 29.5 | 8 |
| 29.8 | 6 |
| 30.6 | 3 |
| 31.1 | 3 |
| 31.3 | 6 |
| 31.5 | 19 |
| 32.1 | 13 |
| 34.0 | 4 |
| 34.8 | 6 |
| 35.9 | 3 |

TABLE 4
Characteristic Peaks of Form 1, example 1

| Angle | Rel. Intensity |
|---|---|
| (°2-theta) | (%) |
| 12.6 | 64 |
| 18.8 | 61 |
| 19.7 | 34 |
| 24.4 | 100 |

### Deuterated Analogues of the Compounds of the Disclosure

General methods/reviews of obtaining metabolite profile and identifying metabolites of a compound are described in: Dalvie, et al., “Assessment of Three Human in Vitro Systems in the Generation of Major Human Excretory and Circulating Metabolites,” Chemical Research in Toxicology, 2009, 22, 2, 357-368, tx8004357 (acs.org); King, R., “Biotransformations in Drug Metabolism,” Ch.3, Drug Metabolism Handbook Introduction, https://doi.org/10.1002/9781119851042.ch3; Wu, Y., et al, “Metabolite Identification in the Preclinical and Clinical Phase of Drug Development,” Current Drug Metabolish, 2021, 22, 11, 838-857, 10.2174/1389200222666211006104502; Godzien, J., et al, “Chapter Fifteen —Metabolite Annotation and Identification”.

Numerous publicly available and commercially available software tools are available to aid in the predictions of metabolic pathways and metabolites of compounds. Examples of such tools include, BioTransofrmer 3.0 (biotransformer.ca/new) which predicts the metabolic biotransformations of small molecules using a database of known metabolic reactions; MetaSite (moldiscovery.com/software/metasite/) which predicts metabolic transformations related to cytochrome P450 and flavin-containing monooxygenase mediated reactions in phase I metabolism; and Lhasa Meteor Nexus (Ihasalimited.org/products/meteor-nexus.htm) offers prediction of metabolic pathways and metabolite structures using a range of machine learning models, which covers phase I and phase II biotransformations of small molecules.

The metabolite profile of Example 1 is evaluated in liver microsomes and hepatocytes (mouse, rat, rabbit, dog, monkey, and human), recombinant human cytochrome P450 enzymes, recombinant human UGT enzymes, and plasma from animals (mouse, rat, and dog). The metabolite profile of Example 1 is comprised of oxidation and glucuronidation. MetaSite (moldiscovery.com/software/metasite/) was used to predicts metabolic transformations related to cytochrome P450 and flavin-containing monooxygenase mediated reactions in phase I metabolism of Example 1-D. Example 1-D1 to Example 1-D24 in Table 5 may afford certain therapeutic advantages resulting from greater metabolic stability, for example increased in vivo half-life, reduced dosage requirements, reduced CYP450 inhibition (competitive or time dependent), or an improvement in therapeutic index or tolerability.

A person with ordinary skill may make additional deuterated analogues of Example 1 with different combinations of Y¹-Y⁵ as provided in Table 5. Such additional deuterated analogues may provide similar therapeutic advantages that may be achieved by the deuterated analogues. The compounds shown in Table 5 are prophetic deuterated analogues (PDA) of Example 1. The PDAs are predicted based on the metabolic profile of Example 1.

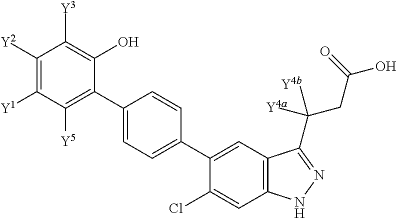

TABLE 5
Prophetic deuterated compounds of Example 1

| Example | | | | | |
|---|---|---|---|---|---|
| Number | Y¹ | Y² | Y³ | Y^{4a}; Y^{4b} | Y⁵ |
| 1-D1 | D | H | H | H | H |
| 1-D2 | H | D | H | H | H |
| 1-D3 | H | H | D | H | H |
| 1-D4 | H | H | H | D | H |
| 1-D5 | H | H | H | H | D |
| 1-D6 | D | D | H | H | H |
| 1-D7 | D | H | D | H | H |
| 1-D8 | D | H | H | D | H |
| 1-D9 | D | H | H | H | D |
| 1-D10 | H | D | D | H | H |
| 1-D11 | H | D | H | D | H |
| 1-D12 | H | D | H | H | D |
| 1-D13 | H | H | D | D | H |
| 1-D14 | H | H | D | H | D |
| 1-D15 | H | H | H | D | D |
| 1-D16 | D | D | D | H | H |
| 1-D17 | D | D | H | D | H |
| 1-D18 | D | D | H | H | D |
| 1-D19 | H | D | D | D | H |
| 1-D20 | H | D | D | H | D |
| 1-D21 | H | H | D | D | D |
| 1-D22 | D | D | D | D | H |
| 1-D23 | D | D | D | H | D |
| 1-D24 | H | D | D | D | D |

### Embodiments

The following non-limiting embodiments provide illustrative examples of the invention, but do not limit the scope of the invention.

Embodiment 1. A compound of Formula (I):

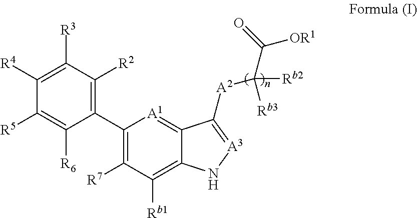

a pharmaceutically acceptable salt, a tautomer, or a pharmaceutically acceptable salt of the tautomer thereof, wherein:
   A¹ is CR⁸, or N;
   A² is CH₂, CHD, CD₂, S, O, or NH;
   A³ is CH, CD, or N;
   R¹ is H, D, C₁₋₈alkyl, C₃₋₆cycloalkyl, or 4-6 membered heterocycloalkyl;
   R², R³, R⁵, and R⁶ are each independently H, D, OH, or halogen;
   R⁴ is monocyclic aryl, bicyclic aryl, monocyclic heteroaryl, or bicyclic heteroaryl, each of which is optionally substituted with R⁹, R¹⁰, R¹¹, R¹², or R¹³,
      wherein R⁹, R¹⁰, R¹¹, R¹², and R¹³ are each independently H, D, halogen, CN, oxo, C₁₋₈alkyl, C₃₋₆cycloalkyl, C₀₋₆alkylene-OR^{x}, C₁₋₆haloalkylene-OR^{x}, C₀₋₆alkylene(C₀₋₆haloalkyl)NR^{x}R^{y}, C₁₋₆alkylene(C₁₋₆haloalkyl)NR^{x}R^{y}, 4-6 membered heterocycloalkyl, C(O)OR^{x}, C₀₋₆alkylene-C(O)NR^{x}R^{y}, OC₁₋₃alkylene-heterocycloalkyl, OC₁₋₃alkylene-C(O)NR^{x}R^{y}, O(C₁₋₆alkyl)SO₂NR^{x}NR^{y}, NR^{x}R^{y}, NHSO₂R^{x}, SR^{x}, S—C₁₋₆alkylene-C(O)NR^{x}R^{y}, S(O)R^{x}R^{y}, SO₂R^{x}, SO₂NR^{x}R^{y}, S(O)(NR^{x})R^{y}, S(O)(NR^{x})R^{y}, or SO₂R^{x};
   wherein each R^{x} and R^{y} is independently H, D, C₁₋₆alkyl, C₁₋₆haloalkyl, C₁₋₆alkoxy, C₃₋₆ cycloalkyl, C₁₋₆alkylene-amide, OC₀₋₂alkylene-heterocycloalkyl, 4-6 membered heterocycloalkyl, C(O)C₁₋₆alkyl, imino, or C₁₋₆alkylsulfonyl; or R^{x} and R^{y} together with the atoms to which R^{x} and R^{y} are bound can form an optionally substituted ring;
   R⁷ is C₁₋₃alkyl, C₃₋₆cycloalkyl, cyano, or halogen;
   R^{b1}, R^{b2}, and R^{b3} are each independently H or D;
   R⁸ is H, D, or halogen; and
   n is 0, 1, or 2

Embodiment 2. The compound of embodiment 1, the pharmaceutically acceptable salt, the tautomer, or the pharmaceutically acceptable salt of the tautomer thereof, wherein A¹ is CH or CF.

Embodiment 3. The compound of embodiment 1 or 2, the pharmaceutically acceptable salt, the tautomer, or the pharmaceutically acceptable salt of the tautomer thereof, wherein A² is CH₂ or S.

Embodiment 4. The compound of any one of embodiments 1-3, the pharmaceutically acceptable salt, the tautomer, or the pharmaceutically acceptable salt of the tautomer thereof, wherein A³ is N.

Embodiment 5. The compound of embodiment 1, the pharmaceutically acceptable salt, the tautomer, or the pharmaceutically acceptable salt of the tautomer thereof, wherein A¹ is N and A³ is N.

Embodiment 6. The compound of any one of embodiments 1-5, the pharmaceutically acceptable salt, the tautomer, or the pharmaceutically acceptable salt of the tautomer thereof, wherein R¹ is H.

Embodiment 7. The compound of any one of embodiments 1-6, the pharmaceutically acceptable salt, the tautomer, or the pharmaceutically acceptable salt of the tautomer thereof, wherein R² is halogen; and R³, R⁵, and R⁶ are each independently H.

Embodiment 8. The compound of any one of embodiments 1-5, the pharmaceutically acceptable salt, the tautomer, or the pharmaceutically acceptable salt of the tautomer thereof, wherein R², R³, R⁵, and R⁶ are each independently H.

Embodiment 9. The compound of any one of embodiments 1-8, the pharmaceutically acceptable salt, the tautomer, or the pharmaceutically acceptable salt of the tautomer thereof, wherein R⁷ is Cl.

Embodiment 10. The compound of any one of embodiments 1-9, the pharmaceutically acceptable salt, the tautomer, or the pharmaceutically acceptable salt of the tautomer thereof, wherein R⁴ is phenyl, wherein R⁹, R¹⁰, R¹¹, R¹², and R¹³ are each independently H, D, Cl, F, CN, C₁₋₃alkyl, C₁₋₆alkylene-OH, C₁₋₆alkylene-OC₁₋₆alkyl, OH, OC₁₋₆alkyl, OC₁₋₆haloalkyl, O(C₁₋₃alkylene)heterocycloalkyl, O(C₁₋₃alkylene)-C(O)NR^{x}R^{y}, C₁₋₃alkylene-NR^{x}R^{y}, C(O)OH, C(O)OC₁₋₃alkyl, C₀₋₂alkylene-C(O)NR^{x}R^{y}, SO₂NR^{x}R^{y}, S(O)(NR^{x})R^{y}, NR^{x}R^{y}, SR^{x}, or SO₂R^{x}.

Embodiment 11. The compound of any one of embodiments 1-10, the pharmaceutically acceptable salt, the tautomer, or the pharmaceutically acceptable salt of the tautomer thereof, wherein R⁴ is 6-membered monocyclic heteroaryl, wherein at least one of R⁹, R¹⁰, R¹¹, R¹², and R¹³ is C₁₋₃alkoxy, halogen, hydroxy, or C(O)NH₂.

Embodiment 12. The compound of embodiment 1, the pharmaceutically acceptable salt, the tautomer, or the pharmaceutically acceptable salt of the tautomer thereof, wherein the compound has the structure Formula (II):

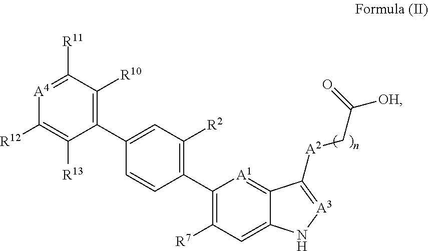

wherein:
R² is H, D, or halogen;
R⁷ is CI or CN;
A² is CH₂, CHD, CD₂, S, or NH;
A⁴ is CR⁹ or N;
R⁹ is H, F, Cl, C₁₋₃alkyl, C₁₋₃alkylene-O—C₁₋₃alkyl, C₁₋₃alkylene-NH₂, COOH, C(O)OC₁₋₃alkyl, C₁₋₃alkylene-C(O)NH₂, C(O)NHC₁₋₃alkyl, C(O)N(C₁₋₃alkyl)₂, C₁₋₆alkylene(C₁₋₆ haloalkyl)NH₂, C₀₋₂alkylene-NH(C(O)C₁₋₃alkyl), OC₁₋₃alkyl, OC₁₋₃haloalkyl, OC. ₃alkylene-heterocycloalkyl, OC₁₋₃alkylene-C(O)NH₂, NHSO₂C₁₋₃alkyl, N(C₁₋₃alkyl)(C(O)C₁₋₃alkyl), SC₁₋₃alkyl, S—C₁₋₃alkylene-C(O)NH₂, SO(NH)C₁₋₃alkyl, SO₂NH₂, or SO₂C₁₋₃alkyl;
R¹⁰ is H, D, or OH;
R¹¹ is H, D, halogen, CN, O(C₁₋₃alkyl), or O(C₁₋₃haloalkyl); or R⁹ and R¹¹ together with the carbon atom to which R⁹ and R¹¹ are bound form an optionally substituted ring;
R¹² is H, OC₁₋₃alkyl, or C₁₋₃alkylene-OH;
R¹³ is H, F, C, or C₁₋₃alkyl; and
n is 1 or 2.

Embodiment 13. The compound of embodiment 12, the pharmaceutically acceptable salt, the tautomer, or the pharmaceutically acceptable salt of the tautomer thereof, wherein R¹⁰ is OH.

Embodiment 14. The compound of embodiment 12 or 13, the pharmaceutically acceptable salt, the tautomer, or the pharmaceutically acceptable salt of the tautomer thereof, wherein R⁷ is C.

Embodiment 15. The compound of any one of embodiments 12-14, the pharmaceutically acceptable salt, the tautomer, or the pharmaceutically acceptable salt of the tautomer thereof, wherein A² is CH₂ or S.

Embodiment 16. The compound of embodiment 1, the pharmaceutically acceptable salt, the tautomer, or the pharmaceutically acceptable salt of the tautomer thereof, wherein the compound has the structure Formula (III):

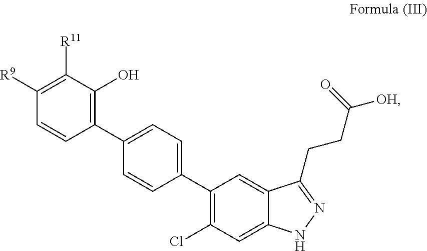

wherein:
R⁹ is H, D, halogen, C₁₋₃alkyl, C(O)NH₂, C₁₋₃alkylene-NH₂, C₁₋₃alkylene-O—C₁₋₃alkyl, C(O)OC₁₋₃alkyl, C(O)OH, OC₁₋₃alkyl, OC₁₋₃haloalkyl, —O(C₁₋₃alkyl)SO₂NH₂, C₁₋₆alkylene(C₁₋₆haloalkyl)NH₂, SC₁₋₃alkyl, or —C(O)NR^{x}R^{y}, wherein each R^{x} and R^{y} are independently H or C₁₋₆alkyl; and
R¹¹ is H, D, halogen, CN, or —O(C₁₋₃alkyl); or R⁹ and R¹¹ together with the carbon atom to which R⁹ and R¹¹ are bound form an optionally substituted ring.

Embodiment 17. The compound of embodiment 16, the pharmaceutically acceptable salt, the tautomer, or the pharmaceutically acceptable salt of the tautomer thereof, wherein:
R⁹ is H or —C(O)NR^{x}R^{y}, wherein each R^{x} and R^{y} is independently H, D, or C₁₋₆alkyl; and
R¹¹ is H or —O(C₁₋₃alkyl); or R⁹ and R¹¹ together with the carbon atom to which R⁹ and R¹¹ are bound form an optionally substituted ring.

Embodiment 18. The compound of embodiment 1, the pharmaceutically acceptable salt, the tautomer, or the pharmaceutically acceptable salt of the tautomer thereof, wherein the compound has the structure Formula (IVa), Formula (IVb), or Formula (IVc):

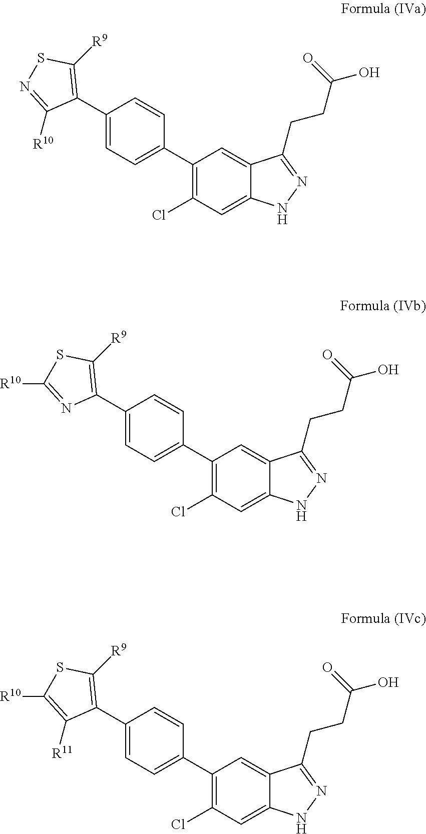

wherein each R⁹, R¹⁰, and R¹¹ are independently H, C₁₋₃alkyl, C₁₋₃alkylene-OH, or OC₁₋₃alkyl.

Embodiment 19. The compound of embodiment 18, or the pharmaceutically acceptable salt, the tautomer, or the pharmaceutically acceptable salt of the tautomer thereof, wherein R⁹, R¹⁰, and R¹¹ are each independently H.

Embodiment 20. The compound of embodiment 18, the pharmaceutically acceptable salt, the tautomer, or the pharmaceutically acceptable salt of the tautomer thereof, wherein R¹⁰ is C₁₋₃alkyl, C₁₋₃alkylene-OH or OC₁₋₃alkyl.

Embodiment 21. The compound of embodiment 1, or the pharmaceutically acceptable salt, the tautomer, or the pharmaceutically acceptable salt of the tautomer thereof, wherein the compound has the structure Formula (V):

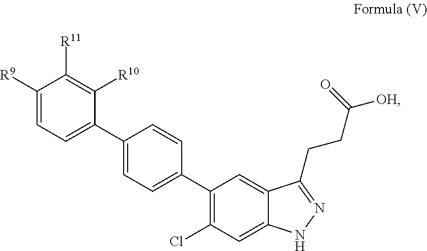

wherein:
R¹⁰ is H, D, or OH;
R⁹ and R¹¹ together with the carbon atom to which R⁹ and R¹¹ are bound form a ring, wherein the ring is 5- or 6-membered heterocycloalkyl or 5- or 6-membered heteroaryl, wherein the ring is optionally substituted with C₁₋₃alkyl, OH, or oxo.

Embodiment 22. The compound of embodiment 21, the pharmaceutically acceptable salt, the tautomer, or the pharmaceutically acceptable salt of the tautomer thereof, wherein the ring is 5-membered heterocycloalkyl.

Embodiment 23. The compound of embodiment 21, the pharmaceutically acceptable salt, the tautomer, or the pharmaceutically acceptable salt of the tautomer thereof, wherein the ring is 5-membered heteroaryl.

Embodiment 24. The compound of any one of embodiments 21-23, the pharmaceutically acceptable salt, the tautomer, or the pharmaceutically acceptable salt of the tautomer thereof, wherein the ring is substituted with C₁₋₃alkyl, OH, or oxo.

Embodiment 25. The compound of embodiment 1, the pharmaceutically acceptable salt, the tautomer, or the pharmaceutically acceptable salt of the tautomer thereof, selected from the group consisting of:
3-(6-chloro-5-(2′-hydroxy-[1,1′-biphenyl]-4-yl)-1H-indazol-3-yl)propanoic acid;
3-(6-chloro-5-(2′-hydroxy-6′-methyl-[1,1′-biphenyl]-4-yl)-1H-indazol-3-yl)propanoic acid;
3-(6-chloro-5-(2′-hydroxy-3′-methoxy-[1,1′-biphenyl]-4-yl)-1H-indazol-3-yl)propanoic acid;
3-(6-chloro-5-(2′-hydroxy-3′-methoxy-6′-methyl-[1,1′-biphenyl]-4-yl)-1H-indazol-3-yl)propanoic acid;
3-(6-chloro-5-(4-(7-hydroxy-2,3-dihydrobenzofuran-6-yl)phenyl)-1H-indazol-3-yl)propanoic acid;
3-(6-chloro-5-(2′-hydroxy-4′-(methoxymethyl)-[1,1′-biphenyl]-4-yl)-1H-indazol-3-yl)propanoic acid;
3-(6-chloro-5-(2′-hydroxy-4′,6′-dimethyl-[1,1′-biphenyl]-4-yl)-1H-indazol-3-yl)propanoic acid;
3-(6-chloro-5-(3′-fluoro-2′-hydroxy-6′-methyl-[1,1′-biphenyl]-4-yl)-1H-indazol-3-yl)propanoic acid;
3-(6-chloro-5-(4′-fluoro-2′-hydroxy-3′-methoxy-[1,1′-biphenyl]-4-yl)-1H-indazol-3-yl)propanoic acid;
3-(6-chloro-5-(4′-(dimethylcarbamoyl)-[1,1′-biphenyl]-4-yl)-1H-indazol-3-yl)propanoic acid;
4-(6-chloro-5-(2′-hydroxy-3′-methoxy-[1,1′-biphenyl]-4-yl)-1H-indazol-3-yl)butanoic acid;
3-(6-chloro-5-(4′-(methylcarbamoyl)-[1,1′-biphenyl]-4-yl)-1H-indazol-3-yl)propanoic acid;
6-((3-(6-chloro-5-(2′-hydroxy-[1,1′-biphenyl]-4-yl)-1H-indazol-3-yl)propanoyl)oxy)-3,4,5-trihydroxytetrahydro-2H-pyran-2-carboxylic acid; and
6-((4′-(3-(2-carboxyethyl)-6-chloro-1H-indazol-5-yl)-[1,1′-biphenyl]-2-yl)oxy)-3,4,5-trihydroxytetrahydro-2H-pyran-2-carboxylic acid.

Embodiment 26. 3-[6-chloro-5-(2′-hydroxy[1,1′-biphenyl]-4-yl)-1H-indazol-3-yl]propanoic acid, a pharmaceutically acceptable salt, a tautomer, or a pharmaceutically acceptable salt of the tautomer thereof.

Embodiment 27. 3-[6-Chloro-5-(2′-hydroxy[1,1′-biphenyl]-4-yl)-1H-indazol-3-yl]propanoic acid.

Embodiment 28. A compound of the structure:

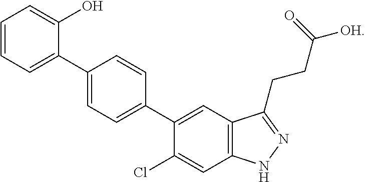 [SMILES: O=C(O)CCc1n[nH]c2cc(Cl)c(-c3ccc(-c4ccccc4O)cc3)cc12]

Embodiment 29. A pharmaceutical composition comprising the compound according to any of embodiments 1 to 28, a pharmaceutically acceptable salt, a tautomer, or a pharmaceutically acceptable salt of the tautomer thereof, and a pharmaceutically acceptable excipient.

Embodiment 30. A method for treating a condition, comprising administering to a subject in need thereof a therapeutically effective amount of the compound of any of embodiments 1 to 28, a pharmaceutically acceptable salt, a tautomer, or a pharmaceutically acceptable salt of the tautomer thereof, wherein the condition is an inflammatory condition, an autoimmune condition, or a functional gastrointestinal disorder.

Embodiment 31. The method of embodiment 30, wherein the inflammatory condition or the autoimmune condition is selected from the group consisting of inflammatory bowel disease, ulcerative colitis, Crohn's disease, celiac disease, atopic dermatitis, psoriasis, rheumatoid arthritis, and lupus.

Embodiment 32. The method of embodiment 30, wherein the functional gastrointestinal disorder is selected from the group consisting of irritable bowel syndrome, functional diarrhea, celiac disease, and functional constipation.

Embodiment 33. The method of any one of embodiments 30-32, wherein the administering is oral.

Embodiment 34. The method of any one of embodiments 30-33, wherein the therapeutically effective amount is from about 1 mg to about 2500 mg.

Embodiment 35. The method of any one of embodiments 30-34, wherein the administering is once a day.

Embodiment 36. The method of any one of embodiments 30-34, wherein the administering is twice a day.

Embodiment 37. A method for treating a condition, comprising:
a) administering to a subject in need thereof a therapeutically effective amount of the compound of any of embodiments 1 to 28, a pharmaceutically acceptable salt, a tautomer, or a pharmaceutically acceptable salt of the tautomer thereof; and
b) administering a therapeutically effective amount of an additional therapeutic agent.

Embodiment 38. The method of embodiment 37, wherein the condition is an inflammatory condition, an autoimmune condition, or a functional gastrointestinal disorder.

Embodiment 39. The method of embodiment 38, wherein the inflammatory condition or the autoimmune condition is selected from the group consisting of inflammatory bowel disease, ulcerative colitis, Colitis, and Crohn's disease.

Embodiment 40. The method of embodiment 38 or 39, wherein the functional gastrointestinal disorder is selected from the group consisting of irritable bowel syndrome, functional diarrhea, celiac disease, and functional constipation.

Embodiment 41. The method of any one of embodiments 37-40, wherein the administering of the compound, or pharmaceutically acceptable salt thereof, is oral.

Embodiment 42. The method of any one of embodiments 37-41, wherein the therapeutically effective amount of the compound, a pharmaceutically acceptable salt, a tautomer, or a pharmaceutically acceptable salt of the tautomer thereof, is from about 1 mg to about 2500 mg.

Embodiment 43. The method of any one of embodiments 37-42, wherein the therapeutically effective amount of the compound, a pharmaceutically acceptable salt, a tautomer, or a pharmaceutically acceptable salt of the tautomer thereof, is from about 1 mg to about 100 mg.

Embodiment 44. A compound according to any of embodiments 1 to 28, a pharmaceutically acceptable salt, a tautomer, or a pharmaceutically acceptable salt of the tautomer thereof, for use as a medicament.

Embodiment 45. A compound according to any of embodiments 1 to 28, a pharmaceutically acceptable salt, a tautomer, or a pharmaceutically acceptable salt of the tautomer thereof, for use in the treatment of an inflammatory condition, an autoimmune condition, or a functional gastrointestinal disorder.

Embodiment 46. Use of a compound according to any of embodiments 1 to 28, a pharmaceutically acceptable salt, a tautomer, or a pharmaceutically acceptable salt of the tautomer thereof, in the manufacture of a medicament for the treatment of an inflammatory condition, an autoimmune condition, or a functional gastrointestinal disorder.

Embodiment 47. Use of a compound according to any one of embodiments 1 to 28, a pharmaceutically acceptable salt, a tautomer, or a pharmaceutically acceptable salt of the tautomer thereof, as a medicament.

Embodiment 48. Use of a compound according to any one of embodiments 1 to 28, a pharmaceutically acceptable salt, a tautomer, or a pharmaceutically acceptable salt of the tautomer thereof, in treating an inflammatory condition, an autoimmune condition, or a functional gastrointestinal disorder.

Embodiment 49. Crystalline 3-[6-Chloro-5-(2′-hydroxy[1,1′-biphenyl]-4-yl)-1H-indazol-3-yl]propanoic acid, a pharmaceutically acceptable salt, a tautomer, or a pharmaceutically acceptable salt of the tautomer thereof.

Embodiment 50. The crystalline compound of embodiment 49 having an X-ray powder diffraction pattern comprising diffraction peaks 12.6±0.2, 18.8 0.2, 19.7 0.2, and 24.4±0.2 degrees two theta.

Each of the embodiments described herein may be combined with any other embodiment(s) described herein not inconsistent with the embodiment(s) with which it is combined. In addition, any of the compounds described in the Examples, or pharmaceutically acceptable salts thereof, may be claimed individually or grouped together with one or more other compounds of the Examples, or pharmaceutically acceptable salts thereof, for any of the embodiment(s) described herein. Furthermore, each of the embodiments described herein envisions within its scope pharmaceutically acceptable salts of the compounds described herein.

It will be apparent to those skilled in the art that various modifications and variations may be made in the present invention without departing from the scope or spirit of the invention. Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

All references cited herein, including patents, patent applications, papers, textbooks, and the like, and the references cited therein, to the extent that they are not already, are hereby incorporated by reference in their entireties. In the event that one or more of the incorporated literature and similar materials differs from or contradicts this application, including but not limited to defined terms, term usage, described techniques, or the like, this application controls.

1. A compound of Formula (I): 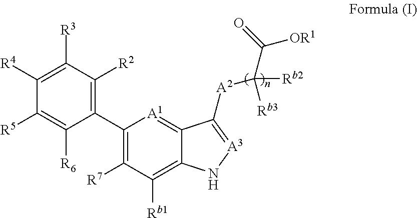
a pharmaceutically acceptable salt, a tautomer, or a pharmaceutically acceptable salt of the tautomer thereof, wherein:
A¹ is CR⁸, or N;
A² is CH₂, CHD, CD₂, S, O, or NH;
A³ is CH, CD, or N;
R¹ is H, D, C₁₋₈alkyl, C₃₋₆cycloalkyl, or 4-6 membered heterocycloalkyl, each of which is optionally substituted;
R², R³, R⁵, and R⁶ are each independently H, D, OH, or halogen;
R⁴ is monocyclic aryl, bicyclic aryl, monocyclic heteroaryl, or bicyclic heteroaryl, each of which is optionally substituted with R⁹, R¹⁰, R¹¹, R¹², or R¹³,
wherein R⁹, R¹⁰, R¹¹, R¹², and R¹³ are each independently H, D, halogen, CN, oxo, C₁₋₈alkyl, C₃₋₆cycloalkyl, C₀₋₆alkylene-OR^{x}, C₁₋₆haloalkylene-OR^{x}, C₀₋₆alkylene(C₀₋₆haloalkyl)NR^{x}R^{y}, C₁₋₆alkylene(C₁₋₆haloalkyl)NR^{x}R^{y}, 4-6 membered heterocycloalkyl, C(O)OR^{x}, C₀₋₆alkylene-C(O)NR^{x}R^{y}, OC₁₋₃alkylene-heterocycloalkyl, OC₁₋₃alkylene-C(O)NR^{x}R^{y}, O(C₁₋₆alkyl)SO₂NR^{x}NR^{y}, NR^{x}R^{y}, NHSO₂R^{x}, SR^{x}, S—C₁₋₆alkylene-C(O)NR^{x}R^{y}, S(O)R^{x}R^{y}, SO₂R^{x}, SO₂NR^{x}R^{y}, S(O)(NR^{x})R^{y}, S(O)(NR^{x})R^{y}, or SO₂R^{x};
wherein each R^{x} and R^{y} is independently H, D, C₁₋₆alkyl, C₁₋₆haloalkyl, C₁₋₆alkoxy, C₃₋₆cycloalkyl, C₁₋₆alkylene-amide, OC₀₋₂alkylene-heterocycloalkyl, 4-6 membered heterocycloalkyl, C(O)C₁₋₆alkyl, imino, or C₁₋₆-alkylsulfonyl; or R^{x} and R^{y} together with the atoms to which R^{x} and R^{y} are bound can form an optionally substituted ring;
R⁷ is C₁₋₃alkyl, C₃₋₆cycloalkyl, cyano, or halogen;
R^{b1}, R^{b2}, and R^{b3} are each independently H or D;
R³ is H, D, or halogen; and
n is 0, 1, or 2.

2. The compound of claim 1, the pharmaceutically acceptable salt, the tautomer, or the pharmaceutically acceptable salt of the tautomer thereof, wherein A¹ is CH or CF.

3. The compound of claim 1, the pharmaceutically acceptable salt, the tautomer, or the pharmaceutically acceptable salt of the tautomer thereof, wherein A² is CH₂ or S.

4. The compound of claim 1, the pharmaceutically acceptable salt, the tautomer, or the pharmaceutically acceptable salt of the tautomer thereof, wherein A³ is N.

5-7. (canceled)

8. The compound of claim 1, the pharmaceutically acceptable salt, the tautomer, or the pharmaceutically acceptable salt of the tautomer thereof, wherein R⁴ is phenyl, wherein R⁹, R¹⁰, R¹¹, R¹², and R¹³ are each independently H, D, Cl, F, CN, C₁₋₃alkyl, C₁₋₆alkylene-OH, C₁₋₆alkylene-OC₁₋₆alkyl, OH, OC₁₋₆alkyl, OC₁₋₆haloalkyl, O(C₁₋₃alkylene)heterocycloalkyl, O(C₁₋₃alkylene)-C(O)NR^{x}R^{y}, C₁₋₃alkylene-NR^{x}R^{y}, C(O)OH, C(O)OC₁₋₃alkyl, C₀₋₂alkylene-C(O)NR^{x}R^{y}, SO₂NR^{x}R^{y}, S(O)(NR^{x})R^{y}, NR^{x}R^{y}, SR^{x}, or SO₂R^{x}.

9. The compound of claim 1, the pharmaceutically acceptable salt, the tautomer, or the pharmaceutically acceptable salt of the tautomer thereof, wherein the compound has the structure Formula (II): 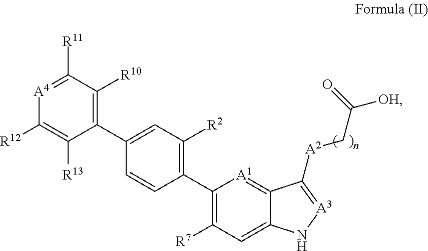
wherein:
R² is H, D, or halogen;
R⁷ is Cl or CN;
A² is CH₂, CHD, CD₂, S, or NH;
A⁴is CR⁹ or N;
R⁹ is H, D, F, Cl, C₁₋₃alkyl, C₁₋₃alkylene-O—C₁₋₃alkyl, C₁₋₃alkylene-NH₂, COOH, C(O)OC₁₋₃alkyl, C₁₋₃alkylene-C(O)NH₂, C(O)NHC₁₋₃alkyl, C(O)N(C₁₋₃alkyl)₂, C₁-₆alkylene(C₁₋₆haloalkyl)NH₂, C₀₋₂alkylene-NH(C(O)C₁₋₃alkyl), OC₁₋₃alkyl, OC₁₋₃haloalkyl, OC₁₋₃alkylene-heterocycloalkyl, OC₁₋₃alkylene-C(O)NH₂, NHSO₂C₁₋₃alkyl, N(C₁-₃alkyl)(C(O)C₁₋₃alkyl), SC₁₋₃alkyl, S—C₁₋₃alkylene-C(O)NH₂, SO(NH)C₁₋₃alkyl, SO₂NH₂, or SO₂C₁₋₃alkyl;
R¹⁰ is H, D, or OH;
R¹¹ is H, D, halogen, CN, O(C₁₋₃alkyl), or O(C₁₋₃haloalkyl); or R⁹ and R¹¹ together with the carbon atom to which R⁹ and R¹¹ are bound form an optionally substituted ring;
R¹² is H, D, OC₁₋₃alkyl, or C₁₋₃alkylene-OH;
R¹³ is H, D, F, C, or C₁₋₃alkyl; and
n is 1 or 2.

10. The compound of claim 9, the pharmaceutically acceptable salt, the tautomer, or the pharmaceutically acceptable salt of the tautomer thereof, wherein R¹⁰ is OH.

11. The compound of claim 9, the pharmaceutically acceptable salt, the tautomer, or the pharmaceutically acceptable salt of the tautomer thereof, wherein R⁷ is Cl.

12. The compound of claim 9, the pharmaceutically acceptable salt, the tautomer, or the pharmaceutically acceptable salt of the tautomer thereof, wherein A² is CH₂ or S.

13. The compound of claim 9, the pharmaceutically acceptable salt, the tautomer, or the pharmaceutically acceptable salt of the tautomer thereof, wherein:
R⁹ is H or —C(O)NR^{x}R^{y}, wherein each R^{x} and R^{y} are independently H or C₁₋₆alkyl; and
R¹¹ is H or —O(C₁₋₃alkyl); or R⁹ and R¹¹ together with the carbon atom to which R⁹ and R¹¹ are bound form an optionally substituted ring.

14. The compound of claim 1, the pharmaceutically acceptable salt, the tautomer, or the pharmaceutically acceptable salt of the tautomer thereof, selected from the group consisting of:
3-(6-Chloro-5-(2′-hydroxy-[1,1′-biphenyl]-4-yl)-1H-indazol-3-yl)propanoic acid;
3-(6-chloro-5-(2′-hydroxy-6′-methyl-[1,1′-biphenyl]-4-yl)-1H-indazol-3-yl)propanoic acid;
3-(6-chloro-5-(2′-hydroxy-3′-methoxy-[1,1′-biphenyl]-4-yl)-1H-indazol-3-yl)propanoic acid;
3-(6-chloro-5-(2′-hydroxy-3′-methoxy-6′-methyl-[1,1′-biphenyl]-4-yl)-1H-indazol-3-yl)propanoic acid;
3-(6-chloro-5-(4-(7-hydroxy-2,3-dihydrobenzofuran-6-yl)phenyl)-1H-indazol-3-yl)propanoic acid;
3-(6-chloro-5-(2′-hydroxy-4′-(methoxymethyl)-[1,1′-biphenyl]-4-yl)-1H-indazol-3-yl)propanoic acid;
3-(6-chloro-5-(2′-hydroxy-4′,6′-dimethyl-[1,1′-biphenyl]-4-yl)-1H-indazol-3-yl)propanoic acid;
3-(6-chloro-5-(3′-fluoro-2′-hydroxy-6′-methyl-[1,1′-biphenyl]-4-yl)-1H-indazol-3-yl)propanoic acid;
3-(6-chloro-5-(4′-fluoro-2′-hydroxy-3′-methoxy-[1,1′-biphenyl]-4-yl)-1H-indazol-3-yl)propanoic acid;
3-(6-chloro-5-(4′-(dimethylcarbamoyl)-[1,1′-biphenyl]-4-yl)-1H-indazol-3-yl)propanoic acid;
4-(6-chloro-5-(2′-hydroxy-3′-methoxy-[1,1′-biphenyl]-4-yl)-1H-indazol-3-yl)butanoic acid;
3-(6-chloro-5-(4′-(methylcarbamoyl)-[1,1′-biphenyl]-4-yl)-1H-indazol-3-yl)propanoic acid;
6-((3-(6-chloro-5-(2′-hydroxy-[1,1′-biphenyl]-4-yl)-1H-indazol-3-yl)propanoyl)oxy)-3,4,5-trihydroxytetrahydro-2H-pyran-2-carboxylic acid; and
6-((4′-(3-(2-carboxyethyl)-6-chloro-1H-indazol-5-yl)-[1,1′-biphenyl]-2-yl)oxy)-3,4,5-trihydroxytetrahydro-2H-pyran-2-carboxylic acid.

15. The compound of claim 1, wherein the compound is 3-(6-chloro-5-(2′-hydroxy-3′-methoxy-[1,1′-biphenyl]-4-yl)-1H-indazol-3-yl)propanoic acid, a pharmaceutically acceptable salt, a tautomer, or a pharmaceutically acceptable salt of the tautomer thereof.

16. The compound of claim 1, wherein the compound is 3-(6-chloro-5-(2′-hydroxy-[1,1′-biphenyl]-4-yl)-1H-indazol-3-yl)propanoic acid, a pharmaceutically acceptable salt, a tautomer, or a pharmaceutically acceptable salt of the tautomer thereof.

17. The compound of claim 1, wherein the compound is 4-(6-chloro-5-(2′-hydroxy-3′-methoxy-[1,1′-biphenyl]-4-yl)-1H-indazol-3-yl)butanoic acid, a pharmaceutically acceptable salt, a tautomer, or a pharmaceutically acceptable salt of the tautomer thereof.

18. The compound of claim 1, wherein the compound is 3-(6-chloro-5-(2′-hydroxy-4′-(methoxymethyl)-[1,1′-biphenyl]-4-yl)-1H-indazol-3-yl)propanoic acid, a pharmaceutically acceptable salt, a tautomer, or a pharmaceutically acceptable salt of the tautomer thereof.

19. The compound of claim 1, wherein the compound is 6-((3-(6-chloro-5-(2′-hydroxy-[1,1′-biphenyl]-4-yl)-1H-indazol-3-yl)propanoyl)oxy)-3,4,5-trihydroxytetrahydro-2H-pyran-2-carboxylic acid, a pharmaceutically acceptable salt, a tautomer, or a pharmaceutically acceptable salt of the tautomer thereof.

20. A pharmaceutical composition comprising the compound of claim 1, a pharmaceutically acceptable salt, a tautomer, or a pharmaceutically acceptable salt of the tautomer thereof, and a pharmaceutically acceptable excipient.

21. A method for treating a condition, comprising administering to a subject in need thereof a therapeutically effective amount of the compound of claim 1, a pharmaceutically acceptable salt, a tautomer, or a pharmaceutically acceptable salt of the tautomer thereof, wherein the condition is an inflammatory condition, an autoimmune condition, or a functional gastrointestinal disorder.

22. The method of claim 21, wherein the inflammatory condition or the autoimmune condition is selected from the group consisting of inflammatory bowel disease, ulcerative colitis, Crohn's disease, celiac disease, atopic dermatitis, psoriasis, rheumatoid arthritis, and lupus.

23. The method of claim 21, wherein the functional gastrointestinal disorder is selected from the group consisting of irritable bowel syndrome, functional diarrhea, celiac disease, and functional constipation.

24-26. (canceled)
